(19) United States
(12) Patent Application Publication
(10) Pub. No.: US 2025/0283846 A1
(43) Pub. Date: Sep. 11, 2025

(54) FLEXIBLE MULTIPARAMETRIC PLANT SENSORS AND METHODS OF MAKING AND USING THEREOF

(71) Applicant: BOARD OF REGENTS, THE UNIVERSITY OF TEXAS SYSTEM, Austin, TX (US)

(72) Inventor: Shawana TABASSUM, Tyler, TX (US)

(73) Assignee: BOARD OF REGENTS, THE UNIVERSITY OF TEXAS SYSTEM, Austin, TX (US)

(21) Appl. No.: 18/861,679

(22) PCT Filed: May 10, 2023
(86) PCT No.: PCT/US2023/021683

§ 371 Date: Oct. 30, 2024

Related U.S. Application Data
(60) Provisional application No. 63/340,166, filed on May 10, 2022.

(51) Int. Cl. G01N 27/327; G01N 27/27; G01N 27/30; G01N 33/00

(52) U.S. Cl. CPC G01N 27/3277; G01N 27/27; G01N 27/308; G01N 33/0098

(57) ABSTRACT

A flexible plant sensor comprising at least one sensor or an array of sensors that are capable of continuous in situ measurement without the use of bioagents and a method of using and making the plant sensor. The plant sensor includes a flexible polymer substrate and one or more sensors disposed on the substrate. The plant sensor includes real time measurement and multipara-metric-correction capabilities.

### CROSS-REFERENCE TO RELATED APPLICATION

This application claims priority benefit of U.S. Provisional Application No. 63/340,166, filed May 10, 2022, which is incorporated herein by reference in its entirety.

### TECHNICAL FIELD

A flexible multiparametric plant sensor capable of real-time continuous in situ plant measurements using bioagent-free sensors.

### BACKGROUND

The world population is estimated to reach 11.2 billion by 2100, while the total cultivable land will not change significantly. The most promising strategy for producing enough food for humans and livestock in the future is to make farms more efficient, profitable, and sustainable in their use of nonrenewable resources. Plants are subjected to biotic (such as microbes, herbivores, invasive plants, and pests attack) and abiotic (drought, flood, salinity, extreme heat/cold, and nutrient deficiencies) stresses throughout their lifecycle. These environmental stresses induce a progressive change in the levels of phytohormones, which circulate throughout the plant via xylem and phloem, and thus, the levels of phytohormones can serve as early signals of plant stress.

The postharvest qualities of fruits and vegetables depend not only on postharvest management practices but also on preharvest monitoring and treatment. Fruits and vegetables that are inappropriately maintained before harvest, can never be improved in quality by any postharvest treatment. Therefore, it is imperative to investigate and control the preharvest factors that are directly associated with the quality of the harvest. Monitoring the progression of plant growth and ripening is crucial to obtain high quality produce and determine the time to harvest. Ripening is a complex process governed by a myriad of factors including hormonal balance. Although the critical role of hormones in plant development has been well established, previous research was mostly centered around how singular hormones affect ripening. For instance, an upsurge in ethylene production is observed in climacteric fruits such as tomatoes and bananas, while a progressive accumulation of the phytohormone abscisic acid is reported in non-climacteric fruits/vegetables such as grapes and bell peppers. However, there is a complex network of hormonal balance and their crosstalk regulates the ripening process. Endogenous salicylic acid (SA) and indole-3-acetic acid (IAA) play multiple roles in plant development and ripening. For instance, their levels are generally higher during the initial phases of development and subside progressively at later stages.

Plants are subjected to biotic (such as microbes, herbivores, invasive plants, and pests attack) and abiotic (drought, flood, salinity, extreme heat/cold, and nutrient deficiencies) stresses throughout their lifecycle. These environmental stresses induce time-dependent biochemical changes, including reduced transpiration and systemic oxidative stress. As a result, there occurs a progressive variation in the levels of phytohormones, which circulate throughout the plant via the xylem and phloem, and thus, the levels of phytohormones can serve as early signals of plant stress. The phytohormones are considered key stress signaling molecules in plants. The primary signal molecules that are produced as a plant's first response to environmental stresses include non-volatile phytohormones such as Salicylic acid (SA), Jasmonic acid (JA), Abscisic acid (ABA), and Indole-3-acetic acid (IAA), and volatile organic compounds including ethylene and terpenoids. SA and JA are the two primary phytohormones released during systemic acquired resistance (a mechanism of protection against a wide variety of stresses), while ethylene and other phytohormones modulate the overall plant response. Alternating levels of SA, JA, and ABA have been proven to be an indicator of drought, salt, and temperature stresses in plants. Likewise, plants release ethylene and terpenoids under abiotic stresses such as drought, salinity, and temperature variations, and biotic stresses such as pests, herbivores, or microbe attacks. Ethylene also regulates fruit ripening and the development and senescence processes in plants. Therefore, accurate and timely measurements of SA, JA, ABA, IAA, and ethylene would aid the producers and scientists in early diagnosing crop stresses before visible symptoms appear and optimizing the resources to minimize stress-induced growth and yield declines in plants.

In addition to phytohormones, stomata (adjustable pores beneath the crop leaves) regulate photosynthesis and other internal processes in plants by controlling gas exchange with the ambient. For instance, transpiration facilitates the release of water vapor by controlling the carbon dioxide intake, oxygen release, and utilization of nutrients. Particularly, transpiration is directly related to the vapor pressure deficit (VPD), which depends on both temperature as well as relative humidity (RH) levels of the ambient and leaf surfaces. Higher VPD results from significantly higher transpiration compared to the translocation of water from the soil to the leaf. As a result, the plant is under water stress and requires more water to utilize the peripheral CO₂. In contrast, a lower VPD value indicates vapor saturation on the leaf surface, which can be a driving factor for fungal infection on leaves. Thus, the VPD is an effective measure of the transportation of water and nutrients from the soil to the leaves. Similar to phytohormone levels, VPD levels are regulated by temperature, humidity, duration of sunlight exposure, and soil water content. Therefore, real-time measurements of VPD are crucial to plant growth monitoring and will aid in managing the rate at which the plants transpire.

There have been no reports of integrated sensors that can provide adequate continuous and real-time hormonal measurements in situ. As a result, the time signatures carried by the chemical species cannot be captured. The most commonly used techniques to measure the phytohormone levels in plants include capillary electrophoresis (CE), high-performance liquid chromatography (HPLC), and nuclear magnetic resonance (NMR) spectroscopy. These methods entail expensive, discrete, disruptive, and time-intensive measurements, and are often not effective until the plants show physical signs of stress. Although enzymatic sensors offer sensitivity and selectivity, they require complex fabrication and have a limited lifetime as the enzyme easily gets denatured. Several bioagent-free sensors have been reported, but these electrodes have a narrow detection range, thereby limiting their application in a wide variety of plants. Other methods to assess the quality and maturity of fruits and vegetables include infrared spectroscopy, imaging and machine vision, and electronic noses. Although these methods provide non-destructive and multiplexed analysis of several internal attributes of the fruit/vegetable, they are discrete, bulky, manually operated, lack spatiotemporal information, and often effective at later stages of ripening.

Thus, there is a need for a flexible multiparametric plant sensor capable of real-time continuous in situ plant measurements using bioagent-free sensors with a wide detection range for various physiological and physical and/or environmental factors associated with plants, wireless data transfer capability, and an energy efficient and low-cost solution, while incurring minimal damage to the plant. The present disclosure provides real-time and in situ monitoring of plants that can differentiate the effect of individual parameters and their combinations on individual plant productivity during its various growth stages and reduce the loss of crops.

### SUMMARY

In one aspect, the present invention is directed to a flexible plant sensor, comprising a flexible substrate; at least one sensor disposed on the flexible substrate selected from a humidity sensor, a temperature sensor, a strain sensor, a pressure sensor, an electrochemical sensor, or a combination thereof, wherein at least one sensor comprises two or more electrodes. In another aspect, the present invention is directed to a flexible plant sensor, comprising one or more flexible substrates; an array of sensors disposed on the one or more flexible substrates comprising: i) an electrochemical sensor and at least one of a temperature sensor and a humidity sensor; and ii) a strain sensor and a pressure sensor.

In some embodiments, the flexible plant sensor may have at least one sensor that has a coefficient of variance between calibration curves of not more than 3% based on four repeating measurements. In some embodiments, the flexible plant sensor may have at least one sensor that has a coefficient of variance between calibration curves of not more than 5% based on four repeating measurements. In certain embodiments, at least one sensor has a coefficient of variance between calibration curves of not more than 8% before and after a dynamic folding test, wherein in the dynamic folding test the flexible plant sensor in an unbent orientation is bent to a 90° angle, returned to the unbent orientation, and repeated up to 30 cycles, or 60 cycles, or 90 cycles, or 100 cycles. In some embodiments, at least one sensor has a hysteresis between 0th and 100th cycles of less than 5%. In some embodiments, at least one sensor has a coefficient of variance of <1%, <2%, <3%, <4%, or <5% up to one hour, or <1%, <2%, <3%, <4%, or <5% up to 7 days.

In some embodiments, the flexible plant sensor is resistant to variations in temperature and at least one sensor has a coefficient of variance between calibration curves of not more than 9% over a temperature range of 10° C. to 60° C. or of 10° C. to 55° C., or <1%, <2%, <3%, <4%, <5%, <6%, <7%, <8%, or <9% over a temperature range of 10° C. to 60° C. or of 10° C. to 55° C. In some embodiments, the flexible plant sensor is resistant to variations in humidity and at least one sensor has a coefficient of variance between calibration curves of not more than 9% over a relative humidity (RH) range of 10 RH to 90 RH, or <1%, <2%, <3%, <4%, <5%, <6%, <7%, <8%, or <9% over a relative humidity (RH) range of 10 RH to 90 RH.

The flexible substrate may be a thermoplastic and/or thermosetting film. In some embodiments, the flexible substrate is a flexible polyimide film, a perfluorinated sulfonic-acid isomer film, or a sulfonated tetrafluoroethylene fluoropolymer-copolymer film.

In some embodiments, the flexible substrate has a thickness of 50 μm to 500 μm, or 100 μm to 400 μm, or 125 μm to 350 μm, or 150 μm to 250 μm, or 175 μm to 200 μm.

The flexible plant sensor may have dimensions suitable to be placed on a plant leaf or a plant stem. In some embodiments, the flexible plant sensor has a width of 0.1 cm to 2 cm, or 0.25 cm to 1.5 cm, or 0.5 cm to 1 cm, or 0.75 cm, and a length of 0.5 cm to 5 cm, or 0.75 cm to 2.5 cm, or 1 cm to 2 cm, or 1.5 cm. In some embodiments, the flexible plant sensor is 5 grams or less. In some embodiments, the flexible plant sensor has a surface area of 1 to 15 cm², or 3 to 12 cm² or 4 to 10 cm², or 6 to 8 cm².

The sensors on the flexible plant sensor may comprise one or more coatings and/or coating compositions. In some embodiments, at least one sensor further comprises a coating selected from a graphene ink, an Ag/AgCl paste, a metal organic framework (MOF), a poly(3,4-ethylenedioxythiophene) polystyrene sulfonate (PEDOT:PSS) cross-linked with 3-glycidyloxypropyl)trimethoxysilane (GOPS), or a combination thereof. The metal organic framework may be at least one metal selected from copper, zinc, or gold. In some embodiments, at least one sensor comprises a coating of CuMOF and carbon black (CB) in a weight ratio of 1:3 to 3:1. In some embodiments, at least one sensor comprises a coating of a composite copper complex (I)-single-walled carbon nanotube coating. In some embodiments, at least one sensor comprises a coating of a functionalized multiwalled carbon nanotube (f-MWCNT) and hydroxyethyl cellulose (HEC) in a weight ratio of 1:6 to 1:1. In some embodiments, at least one sensor comprises a coating of Poly(3,4-ethylenedioxythiophene):poly(styrenesulfonate) (PEDOT:PSS) and 3-(glycidyloxypropyl)trimethoxysilane (GOPS) in a weight ratio of 1:1 to 1:11. In some embodiments, at least one sensor comprises a coating of polydimethylsiloxane (PDMS), deep eutectic solvent (DES), and carbon black (CB) in a weight ratio of 1:1-0.5:0.02-0.04.

The flexible plant sensor may further comprise an encapsulation layer having a thickness of 0.1 to 5.0 mil, or 0.25 to 2.5 mil, or 0.5 mil to 2.0 mil, or 0.75 to 1.5 mil, or 0.80 to 1.0 mil.

In some embodiments, at least one sensor is capable of real-time and/or continuous monitoring of one or more physical and/or chemical parameters in a plant. In some embodiments, the electrochemical sensor is at least one of a phytohormone sensor or a volatile organic compound sensor. In some embodiments, the strain sensor has a gauge factor of at least 800 at a strain of 0.53% to 1.39% and a curvature angle detection of at least 0.018 degrees. In some embodiments, the strain sensor has an angle of curvature detection from 8.98° to 290°. In some embodiments, the volatile organic compound sensor has a detection limit of at least 0.6 ppm. In some embodiments, the phytohormone sensor has a detection range of 1-1000 μM. In some embodiments, the phytohormone sensor comprises a material that is capable of selective oxidation of a target hormone. The phytohormone sensor may be configured to quantitatively measure the levels of at least one of salicylic acid, abscisic acid, jasmonic acid, and indole-3-acetic acid. In some embodiments, the pressure sensor has a detection range of 0.1 kPa to 100 kPa.

In some embodiments, at least one sensor is bioagent-free and/or is non-invasive.

In some embodiments, the flexible plant sensor further comprises a data acquisition system, wherein the data acquisition system comprises a processor; a communication unit; and a power supply unit, and wherein the data acquisition system is in communication with at least one sensor. In some embodiments, the flexible plant sensor further comprises a potentiostat. In some embodiments, the potentiostat in communication with the one or more sensors, and wherein the potentiostat is in communication with one or more of the processor, the communication unit, the power supply unit, and the data acquisition system. In some embodiments, the flexible plant sensor is an electrochemical sensor comprising a reference electrode (RE), a counter electrode (CE), and at least one working electrode (WE).

The flexible plant sensor may include a data acquisition system that comprises a non-transitory computer readable medium, such as memory storage, communicatively coupled to the processor, the non-transitory computer readable medium having stored thereon computer software comprising a set of instructions that, when executed by the processor, causes the electrode control unit to: receive electrode data from each of the three or more electrodes; and send, via the communication unit, the sensor data to an external device. The sensor data may be sent, via the communication unit, to an Internet of Things (IoT) cloud server configured to interact with one or more IoT-capable devices. In some embodiments, the sensor data are sent, via the communication unit, to an external device. The instructions may be configured to select one or more calibration plots to analyze at least one electrode data. In some embodiments, the instructions are configured to perform a signal calibration of at least one sensor. The calibration may comprise a pH-based signal correction, a temperature-based signal correction, a humidity-based signal correction, a sensor bending correction, a pressure-based signal calibration, a signal calibration based on the signal of an analyte, or a combination thereof.

In some aspects, the data acquisition system includes a processor; a communication unit; and a power supply unit. In some aspects, the data acquisition system includes a microcontroller; a communication unit; and a power supply unit. A flexible plant sensor according to any of the foregoing aspects, may further include a voltage booster. In some embodiments, the data acquisition system further comprises a voltage booster. In certain embodiments, the data acquisition system may include one or more processors and memory, which may be coupled together with a bus. The one or more processors and other components may be coupled together with a bus, a separate bus, or may be directly connected together or coupled using a combination of the foregoing. The memory may contain executable code or software instructions that when executed by the one or more processors or processing circuitry cause the one or more processors or processing circuitry to perform the techniques disclosed herein. The memory may be configured to store the one or more calibration plots and/or other instructions.

In some aspects, the data are sent via a wired connection. In some aspects, the data are sent via a wireless connection. In some embodiments, the communication module implements a communication protocol based on Bluetooth or Bluetooth low energy transmission, Wi-Fi, Wi-Max, IEEE 802.11 technology, a radio frequency (RF) communication. In some embodiments, the communication module implements a communication protocol based on general packet radio service (GPRS), enhanced data GSM environment (EDGE), long term evolution-advanced (LTE-A), LTE, 3G, 4G, 5G, code division multiple access (CDMA), wideband CDMA (WCDMA), evolution-data optimized (EVDO), wireless broadband Internet (Wibro), Mobile WiMax, Digital Enhanced Cordless Telecommunications (DECT), Digital AMPS (IS-136/TDMA, Integrated Digital Enhance Network (iDEN), HSPA+, Flash-OFDM, HIPERMAN, WiFi, IBurst, UMTS, W-CDMA, HSPDA+HSUPA, UMTS-TDD and other formats for utilizing cell phone technology, telephony antenna distributions and/or any combinations thereof, and including the use of satellite, microwave technology, the internet, cell tower, telephony and/or public switched telephone network lines. In some embodiments, the communication module implements a communication protocol based on near field communication (NFC).

In some embodiments, at least one sensor is configured to attach to a plant leaf, a plant stem, or both separately or concurrently.

The present invention also includes a kit comprising the plant sensor of any one of claims and a data acquisition system.

Another aspect of the present invention is a method for continuously measuring one or more plant conditions, comprising attaching to the plant an array of sensors, wherein each sensor is operatively connected to data acquisition system; measuring at least one signal correction parameter; and determining a value of the one or more plant conditions using a pre-determined calibration plot, wherein the pre-determined calibration plot is based on the measured value of the at least one signal correction parameter. In some embodiments, the data acquisition system comprises a processor and a communication unit.

In some aspects, the method includes a continuous measurement for at least 120 days, or at least 90 days, or least 60 days, or at least 30 days, or at least two weeks, or at least 10 days, or at least 7 days. In some aspects, the measurement may be conducted over a period of 5 to 30 seconds, 30 to 60 seconds, 1-30 minutes, for example 1 minute to 4 minutes, 1-24 hours, or may be conducted over 24, 36, 48, 60, 72, or more hours. Measurements may be continuously taken for 1, 2, 3, 4, 5, or 6 weeks, or 1, 2, 3, 4, 5, or 6 months. Measurements may be continuously taken for part or all of a growing season.

The sensors according to any of the embodiments described herein may be able to detect peak currents with high stability for long periods of time. In some embodiments, the sensors may show a decrease in peak current detection of less than 10%, 9%, 8%, 7%, 6%, 5%, 4%, 3%, 2%, or 1%, including any values in between the foregoing percentages. In some embodiments, the above stability is maintained over at least one day, at least one week, two or more weeks, at least one month, or one or more months. In some embodiments, a peak current value detected by any of the sensors described herein may have a decrease of 2.5% or less or 1.5% or less over seven days. In some embodiments, SA, IAA, and ET sensors are able to achieve a peak current value showing a decrease of 2.5% or less or 1.5% or less over seven days.

The sensors according to any of the embodiments described herein are highly selective for target analytes relative to interfering species. For example, the sensors according to any of the embodiments described herein will detect a higher signal for target analytes relative to a signal detected from interfering species alone. In some embodiments, the selectivity of the sensors according to any of the embodiments described herein may be at least 50× higher for target analytes relative to a signal corresponding to interfering species. In some embodiments, the selectivity for target analytes is at least 50×, 40×, 30×, 25×, 20×, 19×, 18×, 17×, 16×, 15×, 14×, 13×, 12×, 11×, 10×, 9×, 8×, 7×, 6×, 5×, 4×, 3×, 2×, 1.5×, 1.4×, 1.3×, 1.2×, or 1.1× higher than a signal for one or more interfering species. The selectivity, i.e., signal, for target analytes may be 1.1× to 50× relative to one or more interfering species. In some embodiments, the selectivity for target analytes is any value within the foregoing ranges.

One or more plant conditions data are sent, via the communication unit, to an Internet of Things (IoT) cloud server configured to interact with one or more IoT-capable devices. The data may be sent, via the communication unit, to an external device. Further, one or more calibration plots may be selected to analyze the sensor data. In some embodiments, a signal calibration is performed. The calibration may comprise at least one of a pH-based signal correction, a temperature-based signal correction, a humidity-based signal correction, a sensor bending signal calibration, a pressure-based signal calibration, or a signal calibration based on the signal of an analyte, or a combination thereof. In some embodiments, at least one signal correction parameter is selected from temperature, humidity, sensor bending, pH, and an analyte. In some embodiments, the data acquisition system comprises a potentiostat.

In some aspects, the method includes sending electrode data, via the communication unit, to an Internet of Things (IoT) cloud server configured to interact/communicate with one or more IoT-capable devices. In some aspects, the method includes sending electrode data, via the communication unit, to an external device via a wired connection. In some aspects, the method includes sending electrode data to an external device wirelessly. In some aspects, the external device and/or IoT-capable device may include, but is not limited to, a desktop computer, a laptop computer, typical cell phone, smart device (e.g., smart phones), or similar apparatus including all remote cellular phones using channel access methods defined above (with cellular equipment, public switched telephone network lines, satellite, tower and mesh technology), mobile phones, PDAs, tablets (e.g. refers to all current and future variants, revisions and generations of the Apple IPAD, Samsung Galaxy, HP, Acer, Microsoft, Nook, Google Nexus, Sony, Kindle and all future tablets manufactured by these and other manufactures), Apple IPOD Touch, or a television, watch, timepiece or fob watch and other similar apparatus with WIFI and wireless capability, and controllers having internet or wireless connectivity.

In some embodiments, the one or more calibration plots may be stored on an external device and the data acquisition system may be configured to cause the external device to transmit the one or more calibration plots to the data acquisition system via the cloud server.

Methods according to the claimed invention include continuous measurement for at least 120 days, or at least 90 days, or least 60 days, or at least 30 days, or at least two weeks, or at least 10 days, or at least 7 days.

In some embodiments, the array of sensors is attached to a leaf of the plant, attached to a stem of the plant, or attached to at least two locations of the same plant.

In some embodiments, the method further comprises measuring kinetics of the one or more phytohormones in the plant. In some embodiments, the method further comprises measuring the distribution of the one or more phytohormones in the plant. In some embodiments the method further comprises continuously measuring one or more conditions of one plant or a plurality of plants.

In some embodiments, the method may include at least one of the following in response to the conditions of a plant or a plurality of plants: modifying an irrigation control system, modifying and/or applying a pesticide treatment to a plant, crops, or a plurality of plants, modifying and/or applying a fertilizer or nutrient treatment in response to the conditions of a plant or a plurality of plants, harvesting the plant or a plurality of plants, continuously measuring a second parameter selected from a physical parameter or a chemical parameter. The second parameter may be humidity, temperature, soil conditions, plant growth, pH, or volatile organic compounds.

In some embodiments, the one or more conditions are detected with a deviation of less than 10%, or less than 5%, or less than 1%, or less than 0.5%, or less than 0.1%, wherein the deviation is across at least three repeated measurements.

Another aspect of the invention is directed to a method of manufacturing a flexible plant sensor, comprising: a. attaching a transfer film onto a part of a flexible substrate; b. forming a pattern in the transfer film attached to the flexible substrate, wherein the pattern is not continuously present in the flexible substrate; c. removing a portion of the transfer film on the flexible substrate corresponding to the formed pattern to expose a portion of the flexible substrate; d. applying an ink solution uniformly over the exposed portion of the flexible substrate; e. curing the dispersion solution at 50° C. to 100° C. for 5 minutes to 60 minutes; and f. removing the remaining transfer film from the flexible substrate to obtain the flexible plant sensor. In some embodiments, the method further comprises forming one or more additional patterns in the transfer film by repeating steps b. to e. prior to step f.

In some embodiments, the flexible substrate is a flexible thermoplastic and/or thermosetting film. In some embodiments, the flexible substrate is a flexible polyimide film or a perfluorinated sulfonic-acid isomer based film.

In some embodiments, the step of forming the pattern may comprise cutting the transfer film at a speed of 50 mm/s to 200 mm/s, or 75 mm/s to 150 mm/s, or 80 mm/s to 100 mm/s, or 90 mm/s, or 91 mm/s, or 92 mm/s, or 93 mm/s, or 94 mm/s, or 95 mm/s, or 96 mm/s, or 97 mm/s, or 98 mm/s, or 99 mm/s. In some embodiments, the step of forming the pattern comprises cutting the transfer film using a force of 1.00 N to 6.00 N, or 1.50 N to 5.50 N, or 2.00 N to 5.00 N, or 2.50 N to 4.50 N, or 3.0 N to 4.25 N, or 4.01 N to 4.10 N.

In some embodiments, the ink solution comprises at least one of a sol, a gel, or a paste. In some embodiments, the ink solution comprises a N-Methyl-2-pyrrolidone (NMP), dimethylformamide (DMF), or deionized water. In some embodiments, the ink solution comprises a printable conductive ink. In some embodiments, the printable conductive ink is selected from graphene, reduced graphene oxide, Ag/AgCl, or a combination thereof.

In some embodiments, the method further comprises drop coating one or more additional coatings on the flexible plant sensor.

In some embodiments, a second sensor is formed on the opposite side of the flexible substrate containing a first sensor.

In some embodiments, a first flexible substrate containing a first set of flexible plant sensors may be attached to a second flexible substrate containing a second set of flexible plant sensors using an adhesive.

### BRIEF DESCRIPTION OF DRAWINGS

FIGS. 1A-1B show a schematic illustration of the SPE fabrication process.

FIG. 2 shows Differential Pulse Voltammetry (DPV) responses for the bare and CuMOF-coated electrodes.

FIG. 3A shows DPV responses of the sensor for SA concentrations ranging from 1 μM to 1000 μM. FIG. 3B shows a calibration plot of peak currents for SA (I_{SA}) and CuMOF (I_{CuMOF}).

FIG. 4A shows DPV responses in response to various interfering species. FIG. 4B shows relative signal for the different solutions introduced on the sensor surface.

FIG. 5A shows a cross-section of the sensor installed at the back of the leaf for real-time measurements. FIG. 5B and FIG. 5C show DPV (FIG. 5B) and FTIR responses (FIG. 5C) for the sap collected from unstressed, water- and sunlight-stressed plants. The inset in (c) shows the FTIR peaks for SA.

FIG. 6A shows process flow for preparing the strain sensor. FIG. 6B shows an image of the strain sensor.

FIGS. 7A-7B shows a system architecture schematic (FIG. 7A) and a voltage divider circuit (FIG. 7B).

FIG. 8A shows relative resistance versus bending strain and cylinder diameter for different ambient temperatures. FIG. 8B shows a performance comparison of the MKR100 board with an LCR meter.

FIG. 9 shows relative resistance variations of the strain sensor as a function of time for one bending and unbending cycle.

FIG. 10 shows stem diameter monitoring system including a strain sensor, temperature sensor, and a data acquisition and processing unit in communication with an external device.

FIGS. 11A-11D show Differential Pulse Voltammetry (DPV) characterization of the CuMOF:CB coating for a weight ratio of 1:3 (FIG. 11A), 1:2 (FIG. 11B), 1:1 (FIG. 11C), and 2:1 (FIG. 11D).

FIG. 12A shows Differential Pulse Voltammetry (DPV) responses of CuMOF:CB coating at a weight ratio of 3:1. FIG. 12B shows calibration curves for different weight ratios of CuMOF to CB (1:3, 1:2, 1:1, 2:1, and 3:1).

FIGS. 13A-13C show calibration curves of the humidity sensor for different weight ratios of MWCNT to HEC (1:1, 1:2, 1:4, and 1:6) with the lengths of MWCNT being 10 nm (FIG. 13A), 20 nm (FIG. 13B), and 50 nm (FIG. 13C).

FIG. 14 shows calibration curve of the temperature sensor for different thicknesses of Kapton tape: 0.5 mil, 1 mil and 2 mil.

FIG. 15 shows calibration curves of the temperature sensor for different weight ratios of PEDOT:PSS to GOPS (1:1(G1), 1:3(G3), 1:5(G5), 1:7(G7), 1:9(G9) and 1:11(G11)).

FIG. 16 shows calibration curve of the pressure sensor for different weight ratios of PDMS:DES:CB (1:1:0.02, 1; 0.5:0.02, and 1:0:0.04).

FIG. 17 shows an example of an electrochemical sensor.

FIG. 18A shows FTIR characterization of a CuMOF coating. FIG. 18B shows an SEM image depicting the morphology of the CuMOF/CB/nafion coating over a working electrode of the SA sensor. FIG. 18C shows an NMR plot of the ether mixture of Na[3,5-(CF3)2-pz]. FIG. 18D shows an SEM image of copper complex (I) nanoparticles over a working electrode of an ethylene sensor.

FIG. 19A shows FTIR characterization of functionalized MWCNT. FIG. 19B shows an SEM image depicting the morphology of the HEC/MWCNT/PVPP coating. FIG. 19C shows an SEM image of the porous PDMS indicating the formation of pores due to the evaporation of DES. FIG. 19D shows an SEM image of rGO.

FIG. 20A shows DPV responses of an SA sensor in response to varying concentrations of Salicylic Acid. FIG. 20B shows a calibration curve of the SA sensor indicating the I_{SA}/I_{CuMOF} vs. SA concentrations. FIG. 20C shows CV responses for different concentrations of ethylene.

FIG. 20D shows calibration of ethylene sensor representing the peak current vs. logarithm of the ethylene concentration. Measurements shown in FIGS. 20A-20D were repeated 3 times, with the error bars representing mean and standard error.

FIGS. 21A-21D show calibration curve for a temperature sensor (FIG. 21A), humidity sensor (FIG. 21B), pressure sensor (FIG. 21C), and strain sensor (FIG. 21D). All measurements were repeated 3 times, and the error bars represent mean and standard error.

FIGS. 22A-22F shows reproducibility test of salicylic acid (FIG. 22A), ethylene (FIG. 22B), temperature (FIG. 22C), humidity (FIG. 22D), pressure (FIG. 22E), and strain (FIG. 22F) sensors. All measurements were repeated 3 times, with the error bars representing mean and standard error.

FIGS. 23A-23F shows reproducibility test under cyclic variations of salicylic acid (FIG. 23A), ethylene (FIG. 23B), temperature (FIG. 23C), humidity (FIG. 23D), pressure (FIG. 23E), and strain (FIG. 23F) sensors.

FIGS. 24A-24F shows sensor bending tests for salicylic acid (FIG. 24A), ethylene (FIG. 24B), temperature (FIG. 24C), humidity (FIG. 24D), pressure (FIG. 24E), and strain (FIG. 24F) sensors. All measurements were repeated 3 times, with the error bars representing mean and standard error.

FIGS. 25A-25F shows sensor hysteresis tests for salicylic acid (FIG. 25A), ethylene (FIG. 25B), temperature (FIG. 25C), humidity (FIG. 25D), pressure (FIG. 25E), and strain (FIG. 25F) sensors.

FIGS. 26A-26B show response of salicylic acid (FIG. 26A) and ethylene (FIG. 26B) sensors under varying temperatures. All measurements were repeated 3 times, with the error bars representing mean and standard error.

FIGS. 27A-27C show response of humidity (FIG. 27A), pressure (FIG. 27B), and strain (FIG. 27C) sensors under varying temperatures. All measurements were repeated 3 times, with the error bars representing mean and standard error.

FIGS. 28A-28B show responses of salicylic acid (FIG. 28A) and ethylene (FIG. 28B) sensors under varying humidity levels. All measurements were repeated 3 times, with the error bars representing mean and standard error.

FIGS. 29A-29C show response of humidity (FIG. 29A), pressure (FIG. 29B), and strain (FIG. 29C) sensors under varying humidity levels. All measurements were repeated 3 times, with the error bars representing mean and standard error.

FIGS. 30A-30F show sensors characterized for drift over an hour. All measurements were repeated 3 times, with the error bars representing mean and standard error.

FIGS. 31A-31F show sensors characterized for drift over 12 hours. All measurements were repeated 3 times, with the error bars representing mean and standard error.

FIGS. 32A-32B show optical images of unstressed (FIG. 32A) and water-stressed (FIG. 32B) bell pepper plants taken after 40 days of measurements.

FIGS. 33A-33C depict a SA sensor coated with CuMOF:CB:Nafion (FIG. 33A), ethylene sensor coated with a Cu complex selective layer (FIG. 33B), and temperature, humidity, pressure, and strain sensors, each coated with the respective selective layer (FIG. 33C).

FIGS. 34A-34B show singular Value Deposition (FIG. 34A) and cumulative Energy of different principal components (FIG. 34B).

FIGS. 35A-35F show principal component analysis for different stress stages of tomato plants scatter plot of principal component 1 versus principal component 2 (FIG. 35A) and a scatter plot of principal component 3 versus principal component 4 (FIG. 35B). Principal component analysis for different growth stages of tomato plants scatter plot of principal component 1 versus principal component 2 (FIG. 35C) and a scatter plot of principal component 3 versus principal component 4 (FIG. 35D). Normalized cross-correlation coefficients between SA and ET (FIG. 35E), SA and VPD (FIG. 35F), and ET and VPD (FIG. 35G), in water-stressed bell pepper plants.

FIGS. 36A-36D show a plot of relative signal in response to different solutions introduced to the SA sensor (FIG. 36A). FIG. 36B shows a calibration plot of the SA sensor in presence of 50 μM of glucose, soluble starch, L-tryptophan, L-cysteine, ABA, GA, JA, OA, IAA, and CA. FIG. 36C shows Current difference (in μA) from the baseline for the ethylene sensor. FIG. 36D shows a calibration plot of the ethylene sensor in presence of 50 ppm of N₂, CH₄, N₂O, and NH₃. All measurements were repeated 3 times, with the error bars representing mean and standard error.

FIGS. 37A-37F show long-term stability of the six sensors evaluated by testing the sensors over a week. All measurements were repeated 3 times, with the error bars representing mean and standard error.

FIGS. 38A-38C show continuous measurements of VPD, SA levels, and ethylene levels, respectively, in unstressed plants and in water stressed plants kept in sunlight.

FIGS. 39A-39C show continuous measurements of VPD, SA levels, and ethylene levels, respectively, in unstressed plants and in unstressed plants kept in shade.

FIGS. 40A-40B show a microscopic image of open stomata from the leaf of an unstressed plant (FIG. 40A) and closed stomata from the leaf of a water stressed plant (FIG. 40B).

FIG. 41A shows humidity measurements at three locations (low, mid, and upper leaves) of the plant. FIG. 41B shows SA measurements at two locations (lower and upper leaves) of the plant. FIG. 41C shows ethylene measurements at two locations (lower and upper leaves) of the plant. FIGS. 41D-41F shows humidity, SA, and ethylene measurements, respectively, at two different locations of the same leaf (75 cm from ground): one sensor was placed closer to the stem and the other 5 cm away near the tip of the leaf. Sensors were placed at three leaves, located at Low=40 cm, Mid=75 cm, and Upper-105 cm from the ground.

FIGS. 42A-42B show continuous measurements of SA and ethylene levels, respectively, in the leaf under periodic water stress and irrigation. Measurements were repeated with 15 stressed plants and 15 control plants, with error bars representing the standard deviation. FIGS. 42C-42D show cross correlation plots of SA and ethylene levels, respectively, at different days.

FIGS. 43A-43F show the fabrication procedure for the salicylic acid sensor, ethylene sensor, temperature sensor, humidity sensor, and pressure sensor.

FIGS. 44A-44E show the fabrication procedure for the strain sensor.

FIGS. 45A-45C show the fabrication process for an electrode suite. FIG. 45A shows the 3D printed microneedle electrodes. An exemplary screen printing process for an ethylene sensor is depicted in FIG. 45B. FIG. 45C depicts a sensor suite attached to a plant and interfaced with a drone.

FIGS. 46A-46D show plots for electrochemical measurements using differential pulse voltammetry (DPV) for SA and IAA. Differential Pulse Voltammetry (DPV) responses for different concentrations of SA are shown in FIG. 46A. A SA calibration curve showing I_{SA}/I_{CuMOF} vs. SA concentrations is shown in FIG. 46B. DPV responses for different concentrations of IAA are shown in FIG. 46C. An IAA calibration curve showing I_{1AA} vs. IAA concentrations is shown in FIG. 46D.

FIGS. 47A-47D show plots for Cyclic Voltammetry (CV) measurements used to conduct electrochemical characterization of an ethylene sensor. CV responses for different concentrations of ethylene are shown in FIG. 47A. An ethylene calibration curve showing current vs. ethylene concentrations are shown in FIG. 47B. CV responses for PANI deposition on an electrode is shown in FIG. 47C. A plot for a pH sensor calibration curve is shown in FIG. 47D. Error bars represent 3 repeated measurements.

FIGS. 48A-48C show plots for a selectivity test for SA (FIG. 48A) and IAA (FIG. 48B) sensors. A plot for Selectivity test for ethylene sensor is shown in FIG. 48C.

FIGS. 49A-49B show calibration curves of SA (FIG. 49A) and IAA (FIG. 49B) sensors for different pH conditions.

FIGS. 50A-50D show trends of SA and IAA levels in unripe (FIG. 50A) and ripe (FIG. 49B) bell peppers. FIG. 50C shows trends of ethylene in ripe and unripe bell peppers. FIG. 50D shows a plot of stability for SA, IAA, and ET sensors over one week.

FIG. 51 depicts exemplary system architecture for a sensor suite.

FIGS. 52A-52B depict Gauge Factor (GF) versus bending strain (FIG. 52A) and a motorized base set-up to measure the bending radius of the strain sensor (FIG. 52B).

FIGS. 53A-53F depict dynamic response of an SA sensor (FIG. 53A), ET sensor (FIG. 53B), temperature sensor (FIG. 53C), relative humidity sensor (FIG. 53D), pressure sensor (FIG. 53E), and strain sensor (FIG. 53F).

FIGS. 54A-54F show calibration plots of the modified sensor DPV responses of the SA sensor in response to varying concentrations of salicylic acid (FIG. 54A). Calibration curve of the SA sensor indicating the plot of I_{SA}/I_{CuMOF} against SA concentrations (FIG. 54B). CV responses of the ethylene sensor for different concentrations of gaseous ethylene (FIG. 54C). Calibration curve of ethylene sensor representing the peak current vs. logarithm of the ethylene concentration (FIG. 54D). Calibration curves for temperature (FIG. 54E) and humidity (FIG. 54F) sensors.

FIGS. 55A-55B show real-time SA and ethylene measurements (FIG. 55A) and real-time VPD measurements from the tomato seedlings starting from day 5 to 20 of their growth period (FIG. 55B).

FIGS. 56A-56F show 10 days of VPD measurements from control (FIG. 56A) and water-stressed (FIG. 56B) plants; 10 days of leaf relative humidity measurements from control (FIG. 56C) and water-stressed (FIG. 56D) plants; and 10 days of leaf temperature measurements from control (FIG. 56E) and water-stressed (FIG. 56F) plants.

FIGS. 57A-57H show a comparison of the SA levels measured with sensors against the values from high-performance liquid chromatography in control (FIG. 57A) and water-stressed (FIG. 57B) bell pepper plants kept in sunlight, and control (FIG. 57C) and water-stressed (FIG. 57D) bell pepper plants kept in the shade over 40 days, a comparison of the SA levels measured with the sensors against the values from high-performance liquid chromatography in (FIG. 57E) control and water-stressed (FIG. 57F) cabbage plants over 2 months, and a comparison of the ethylene levels estimated with our sensor against known concentrations of pure ethylene (FIG. 57G) and ethylene mixed with interfering gases (i.e., N₂, CH₄, N₂O, and NH₃) (FIG. 57H).

FIGS. 58A-58B show a comparative analysis of the kinetics of SA and VPD at the lower leaf (FIG. 58A; located at 40 cm from the soil surface) and upper leaf (FIG. 58B; located at 105 cm from the soil surface) of a bell pepper plant.

FIGS. 59A-59B show optical images of tomato plants at different stages of growth, i.e., 5, 10, 15, and 20 days after germination (FIG. 59A) and the reconfigured sensor suite installed on the leaf of a 15-day-old tomato plant with combined SA, ethylene, temperature and humidity sensors fit into the leaf that was 9.5 mm long and 4.5 mm wide near the center (FIG. 59B).

### DETAILED DESCRIPTION

While aspects of the subject matter of the present disclosure may be embodied in a variety of forms, the following description is merely intended to disclose some of these forms as specific examples of the subject matter encompassed by the present disclosure. Accordingly, the subject matter of this disclosure is not intended to be limited to the forms or embodiments so described.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are in weight, unless specified otherwise.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of,” as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e. “comprising,” “consisting,” or “consisting essentially”) provides direct support for replacement to any of the other transition term not specifically used. For example, amending a term from “comprising” to “consisting essentially of” or “consisting of” would find direct support due to this definition for any elements disclosed throughout this disclosure. Based on this definition, any element disclosed herein or incorporated by reference may be included in or excluded from the claimed invention.

As used herein, a plurality of compounds, elements, or steps may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

A “plant sensor” means a device configured to attach to a plant and to sense one or more conditions, including, but not limited to, pH, temperature, water content, or humidity and/or one or more analytes, including, but not limited to phytohormones, phytochemicals, or volatile organic compounds. In some embodiments, the plant sensor may be a plant patch or a leaf patch, such as a microneedle patch, configured to attach to a part of a plant, such as a plant stem or one or more leaves. In some embodiments, the plant sensor is flexible.

A “plant sensor system” means one or more components in addition to the plant sensor, including, but not limited to, a potentiostat, one or more of the processors, a communication unit, a power supply unit, and/or a data acquisition system. A plant sensor system may include additional components to perform any of the techniques described herein.

A “processor” means one or more microprocessors, central processing units (CPUs), processing circuitry, computing devices, one or more microcontrollers, digital signal processors, or like devices or any combination thereof, regardless of the architecture (e.g., chip-level multiprocessing/multi-core, RISC, CISC, Microprocessor without Interlocked Pipeline Stages, pipelining configuration, simultaneous multithreading). In some embodiments, the processor is operatively connected to memory. The processor and memory may be connected externally or internally.

“Bioagent” means a biological substance, including, but not limited to peptides, enzymes, polypeptides and proteins, nucleotides/nucleic acid, or polynucleotides, any organism, cell, or virus, living or dead, or a nucleic acid derived from such an organism, cell or virus.

“Non-invasive” means plant tissue is minimally affected by any sensors or measurements. In some embodiments, a non-invasive sensor is attached to the plant without any damage, for example without penetrating the tissue of the plant, including the leaf, stem, or roots. In some embodiments, a non-invasive sensor is attached to the plant with minimal damage. In some embodiments, a non-invasive sensor is configured to penetrate plant tissue by a few microns in depth. In some embodiments, a non-invasive sensor is configured to penetrate plant tissue by 1-100 micrometers, or a non-invasive sensor is configured to penetrate plant tissue by less than 90, 80, 70, 60, 50, 40, 30, 20, 10, 9, 8, 7, 6, 5, 4, 3, 2, or 1 micrometers.

“Biocompatible polymer” means any synthetic (man made) or natural polymers which are suitable to be used in the close vicinity of a living system or work in intimacy with living tissue. Examples of biocompatible polymers include, but are not limited to, polyethylenes, polyvinyl chlorides, polyamides, such as nylons, polyesters, rayons, polypropylenes, polyacrylonitriles, auylics, polyisoprenes, polybufadienes and polybutadiene-polyisoprene copolymers, neoprenes and nitrile rubbers, polyisobutylenes, olefinic rubbers, such as ethylene-propylene rubbers, ethylene-propylene-diene monomer rubbers, and polyurethane elastomers, silicone rubbers, fluoroelastomers and fluorosilicone rubbers, homopolymers and copolymers of vinyl acetates, such as ethylene vinyl acetate copolymer, homopolymers and copolymers of acrylates, such as polymethylmethacrylate, polymethylmethacrylate, polymethacrylate, ethylene glycol dimethacrylate, ethylene dimethacrylate and hydroxymethyl methacrylate, polyvinylpyrrolidones, polyacrylonitrile butadienes, polycarbonates, polyamides, fluoropolymers, such as polytetrafluoroethylene and polyvinyl fluoride, polystyrenes, homopolymers and copolymers of styrene acrylonitrile, cellulose acetates, homopolymers and copolymers of acrylonitrile butadiene styrene, polymethylpentenes, polysulfones, polyesters, polyimides, polyisobutylenes, polymethylstyrenes, and other similar compounds known to those skilled in the art.

In some embodiments, the biocompatible polymer comprises a photopolymer resin. In some embodiments, the biocompatible polymer comprises a mixture of methacrylic acid esters and a photoinitiator.

“Computer-readable medium” means any medium, a plurality of the same, or a combination of different media, that participate in providing data (e.g., instructions, data structures) which may be read by a computer, a processor or a like device. Such a medium may take many forms, including but not limited to, non-volatile media, volatile media, and transmission media. Non-volatile media include, for example, optical or magnetic disks and other persistent memory. Volatile media include random access memory (RAM) or dynamic random access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, SecureDigital (SD™) memory card, USB Flash Drives, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read.

A “drone” means a device that is autonomous or unmanned, such that it does not have a human operator onboard. A drone may include, but is not limited to, an unmanned aerial vehicle (UAV), unmanned ground vehicle (UGV), or an unmanned stationary device. The drone may include an interface for receiving, stowing, providing one or more items, or connecting and/or communicating by means of data or information transfer to designated items, for example one or more sensors. The interface may include active and/or passive mechanisms that couple to an item and secure the item with the drone during transportation in flight or on the ground. In addition, the mechanisms may decouple the item from the drone upon reaching a designated destination, e.g., to provide the item at a designated location, to pick up an item at a designated location. In an embodiment, the mechanism includes an actuated mechanical arm with a grip interface to attach and de-attach the item. The drone may include any number of sensors for data collection, navigation, landing, or other functionality. Additionally, the drone may include one or more motors (e.g., electric motors) for actuating one or more rotors, wheels, tracks, or other means of travel. In some embodiments, the drone may include more than one motor. An onboard battery, which may be rechargeable, provides power for the motors as well as other functionality of the drone. The drone may be configured to operate remotely without a wired connection or via a wired connection.

The primary signal molecules that are produced as a plant's first response to environmental stresses include non-volatile phytohormones such as salicylic acid (SA), Jasmonic Acid (JA), Abscisic acid (ABA), and Indole-3-acetic acid (IAA), and volatile organic compounds including ethylene and terpenoids. SA and JA are the two primary phytohormones released during systemic acquired resistance (a mechanism of protection against a wide variety of stresses), while ethylene and other phytohormones modulate the overall plant response. Alternating levels of SA, JA, and ABA have been proved to be an indicator of drought, salt, and temperature stresses in plants. Likewise, plants release ethylene and terpenoids under abiotic stresses such as drought, salinity, temperature variations, and biotic stresses such as pests, herbivores, or microbe attacks. Ethylene also regulates fruit ripening and the development and senescence processes in plants. Therefore, accurate and timely measurements of SA, JA, ABA, IAA, and ethylene would aid the producers in early diagnosing crop stresses before visible symptoms appear and taking immediate steps to mitigate the productivity losses.

In addition to phytohormones, stomata (adjustable pores beneath the crop leaves) regulate photosynthesis and other internal processes in plants by controlling gas exchange with the ambient. For instance, transpiration facilitates the release of water vapor by controlling the carbon dioxide intake, oxygen release, and utilization of nutrients. Particularly, transpiration is directly related to the vapor pressure deficit (VPD), which depends on both temperature as well as relative humidity (RH) levels of the ambient and leaf surfaces. Higher VPD results from significantly higher transpiration compared to the translocation of water from the soil to the leaf. As a result, the plant is under water stress and requires more water to utilize the peripheral CO₂. In contrast, a lower VPD value indicates vapor saturation on the leaf surface, which can be a driving factor for fungal infection on leaves. Thus, the VPD is an effective measure of the transportation of water and nutrients from the soil to the leaves. Similar to the phytohormone levels, VPD levels are regulated by temperature, humidity, duration of sunlight exposure, and soil water content. Therefore, real-time measurements of VPD are crucial to plant growth monitoring.

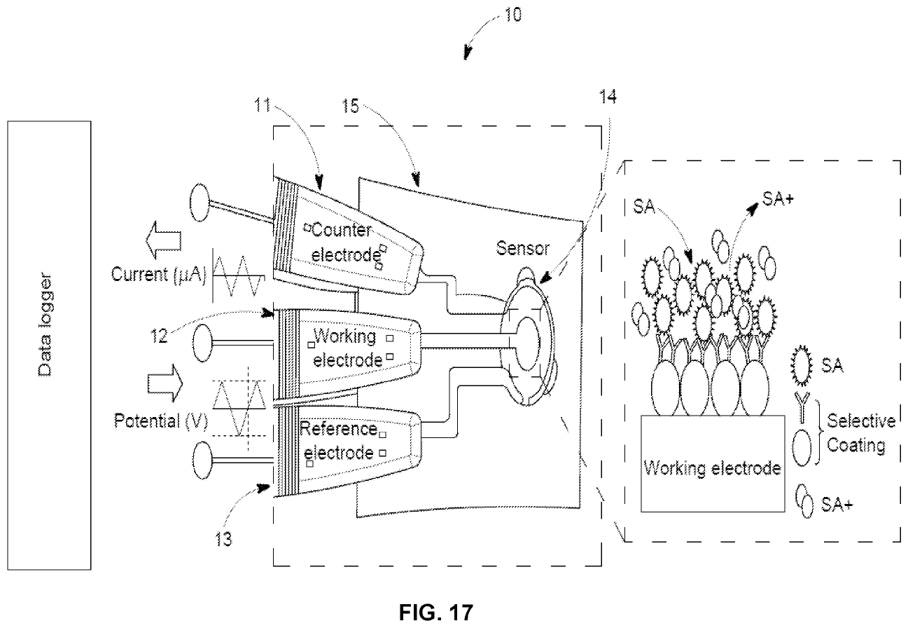

FIG. 17 shows a non-limiting specific embodiment of a hormone sensor. The electrochemical hormone sensor 10 used in the examples as noted below to detect Salicylic Acid. The sensor 14, disposed on a substrate 15, relied on electrochemistry wherein redox reaction of SA on the chemically functionalized working electrode 12 was translated to a current flow proportional to the hormone concentration. The working electrode 12 was coated with a CuMOF:CB layer selective to SA. The data logger (not shown), comprising the Emstat potentiostat used in the examples, sent a voltage pulse between the working and reference electrodes (12, 13) of the sensor. As a result, SA was oxidized on the working electrode 12 and formed SA+, while the CuMOF was reduced. This redox reaction generated a current flow between the working and counter electrodes (11, 12), recorded and analyzed by the data logger.

In some embodiments, the present disclosure includes any one or combination of the following non-limiting numbered items:

1. A flexible plant sensor, comprising:
a flexible substrate;
at least one sensor disposed on the flexible substrate selected from a humidity sensor, a temperature sensor, a strain sensor, a pressure sensor, an electrochemical sensor, or a combination thereof,
wherein the at least one sensor comprises two or more electrodes.

2. A flexible plant sensor, comprising:
one or more flexible substrates;
an array of sensors disposed on the one or more flexible substrates comprising:
i) an electrochemical sensor and at least one of a temperature sensor and a humidity sensor; and
ii) a strain sensor and a pressure sensor.

3. The flexible plant sensor of any one of items 1 or 2, wherein the at least one sensor has a coefficient of variance between calibration curves of not more than 3% based on four repeating measurements.

4. The flexible plant sensor of any one of items 1 or 2, wherein the at least one sensor has a coefficient of variance between calibration curves of not more than 5% based on four repeating measurements.

5. The flexible plant sensor of any one of items 1-4, wherein the at least one sensor has a coefficient of variance between calibration curves of not more than 8% before and after a dynamic folding test, wherein in the dynamic folding test the flexible plant sensor in an unbent orientation is bent to a 90° angle, returned to the unbent orientation, and repeated up to 30 cycles, or 60 cycles, or 90 cycles, or 100 cycles.

6. The flexible plant sensor of any one of items 1-5, wherein the at least one sensor has a hysteresis between 0th and 100th cycles of less than 5%.

7. The flexible plant sensor of any one of items 1-6, wherein the at least one sensor has a coefficient of variance of <5% up to one hour, or <5% up to 7 days.

8. The flexible plant sensor of any one of items 1-7, wherein the at least one sensor has a coefficient of variance between calibration curves of not more than 9% over a temperature range of 10° C. to 55° C.

9. The flexible plant sensor of any one of items 1-8, wherein the at least one sensor has a coefficient of variance between calibration curves of not more than 9% over a relative humidity (RH) range of 10 RH to 90 RH.

10. The flexible plant sensor of any one of items 1-9, wherein the flexible substrate is a thermoplastic and/or thermosetting film.

11. The flexible plant sensor of any one of items 1-10, wherein the flexible substrate is a flexible polyimide film, a perfluorinated sulfonic-acid isomer film, or a sulfonated tetrafluoroethylene fluoropolymer-copolymer film.

12. The flexible plant sensor of any one of items 1-11, wherein the flexible substrate has a thickness of 50 μm to 500 μm, or 100 μm to 400 μm, or 125 μm to 350 μm, or 150 μm to 250 μm, or 175 μm to 200 μm.

13. The flexible plant sensor of any one of items 1-12, having a width of 0.1 cm to 2 cm, or 0.25 cm to 1.5 cm, or 0.5 cm to 1 cm, or 0.75 cm, and a length of 0.5 cm to 5 cm, or 0.75 cm to 2.5 cm, or 1 cm to 2 cm, or 1.5 cm.

14. The flexible plant sensor of any one of items 1-13, wherein flexible plant sensor is 5 grams or less.

15. The flexible plant sensor of any one of items 1-14, wherein flexible plant sensor has a surface area of 1 to 15 cm², or 3 to 12 cm² or 4 to 10 cm², or 6 to 8 cm².

16. The flexible plant sensor of any one of items 1-15, wherein the at least on sensor further comprises a coating selected from a graphene ink, an Ag/AgCl paste, a metal organic framework (MOF), a poly(3,4-ethylenedioxythiophene) polystyrene sulfonate (PEDOT:PSS) cross-linked with 3-glycidyloxypropyl)trimethoxysilane (GOPS), or a combination thereof.

17. The flexible plant sensor of any one of items 1-16, wherein the metal organic framework comprises at least one metal selected from copper, zinc, or gold.

18. The flexible plant sensor of any one of items 1-17, wherein the at least one sensor comprises a coating of CuMOF and carbon black (CB) in a weight ratio of 1:3 to 3:1.

19. The flexible plant sensor of any one of items 1-18, wherein the at least one sensor comprises a coating of a composite copper complex (I)-single-walled carbon nanotube coating.

20. The flexible plant sensor of any one of items 1-19, wherein the at least one sensor comprises a coating of a functionalized multiwalled carbon nanotube (f-MWCNT) and hydroxyethyl cellulose (HEC) in a weight ratio of 1:6 to 1:1.

21. The flexible plant sensor of any one of items 1-20, wherein the at least one sensor comprises a coating of Poly(3,4-ethylenedioxythiophene):poly(styrenesulfonate) (PEDOT:PSS) and 3(glycidyloxypropyl)trimethoxysilane (GOPS) in a weight ratio of 1:1 to 1:11.

22. The flexible plant sensor of any one of items 1-21, wherein the at least one sensor comprises a coating of polydimethylsiloxane (PDMS), deep eutectic solvent (DES), and carbon black (CB) in a weight ratio of 1:1-0.5:0.02-0.04.

23. The flexible plant sensor of any one of items 1-22, wherein the at least one sensor further comprises an encapsulation layer having a thickness of 0.5 mil to 2 mil.

24. The flexible plant sensor of any one of items 1-23, wherein the at least one sensor is capable of real-time and/or continuous monitoring of one or more physical and/or chemical parameters in a plant.

25. The flexible plant sensor of any one of items 1-24, wherein the electrochemical sensor is at least one of a phytohormone sensor or a volatile organic compound sensor.

26. The flexible plant sensor of any one of items 1-25, wherein the strain sensor has a gauge factor of at least 800 at a strain of 0.4% to 2% and a curvature angle detection of at least 0.01 degrees.

27. The flexible plant sensor of any one of items 1-26, wherein the strain sensor has an angle of curvature detection from 5° to 350° or 6° to 320° or 7° to 300° or 8° to 290°.

28. The flexible plant sensor of any one of items 1-27, wherein the volatile organic compound sensor has a detection limit of at least 0.6 ppm.

29. The flexible plant sensor of any one of items 1-28, wherein the phytohormone sensor has a detection range of 1-1000 μM.

30. The flexible plant sensor of any one of items 1-29, wherein the phytohormone sensor comprises a material that is capable of selective oxidation of a target hormone.

31. The flexible plant sensor of any one of items 1-30, wherein the phytohormone sensor is configured to quantitatively measure the level of at least one of salicylic acid, abscisic acid, jasmonic acid, and indole-3-acetic acid.

32. The flexible plant sensor of any one of items 1-31, wherein the pressure sensor has a detection range of 0.1 kPa to 100 kPa.

33. The flexible plant sensor of any one of items 1-32, wherein the at least one sensor is bioagent-free.

34. The flexible plant sensor of any one of items 1-33, wherein the at least one sensor is non-invasive.

35. The flexible plant sensor of any preceding item, further comprising a data acquisition system, wherein the data acquisition system comprises a processor; a communication unit; and a power supply unit, and wherein the data acquisition system is in communication with the at least one sensor.

36. The flexible plant sensor of any preceding item, further comprising a potentiostat.

37. The flexible plant sensor of any preceding item, wherein the electrochemical sensor comprises a reference electrode (RE), a counter electrode (CE), and at least one working electrode (WE).

38. The flexible plant sensor of any preceding item, wherein the data acquisition system comprises a non-transitory computer readable medium communicatively coupled to the processor, the non-transitory computer readable medium having stored thereon computer software comprising a set of instructions that, when executed by the processor, causes the electrode control unit to:
receive electrode data from each of the three or more electrodes; and
send, via the communication unit, the sensor data to an external device.

39. The flexible plant sensor of any preceding item, wherein the sensor data are sent, via the communication unit, to an Internet of Things (IoT) cloud server configured to interact with one or more IoT-capable devices.

40. The flexible plant sensor of any preceding item, wherein the sensor data are sent, via the communication unit, to an external device.

41. The flexible plant sensor of any preceding item, wherein the flexible plant sensor is configured to select one or more calibration plots to analyze at least one electrode data.

42. The flexible plant sensor of any preceding item, wherein the flexible plant sensor is configured to perform a signal calibration of at least one sensor.

43. The flexible plant sensor of any preceding item, wherein the calibration comprises a pH-based signal correction, a temperature-based signal correction, a humidity-based signal correction, a sensor bending correction, a pressure-based signal calibration, a signal calibration based on the signal of an analyte, or a combination thereof.

44. The flexible plant sensor of any preceding item, wherein the at least one sensor is configured to attach to a plant leaf, a plant stem, or both separately or concurrently.

45. A kit comprising the plant sensor of any one of items and a data acquisition system.

46. A method for continuously measuring one or more plant conditions, comprising: attaching to the plant an array of sensors, wherein each sensor is operatively connected to data acquisition system;
measuring at least one signal correction parameter;
determining a value of the one or more plant conditions using a pre-determined calibration plot, wherein the pre-determined calibration plot is based on the measured value of the at least one signal correction parameter.

47. The method of item 46, wherein the at least one signal correction parameter is selected from temperature, humidity, sensor bending, pH, and an analyte.

48. The method of items 46-47, wherein the data acquisition system comprises a potentiostat.

49. The method of items 46-48, wherein the continuous measurement occurs for at least 120 days, or at least 90 days, or least 60 days, or at least 30 days, or at least two weeks, or at least 10 days, or at least 7 days.

50. The method of items 46-49, wherein the data acquisition system comprises a processor and a communication unit.

51. The method of items 46-50, wherein one or more plant conditions data are sent, via the communication unit, to an Internet of Things (IoT) cloud server configured to interact with one or more IoT-capable devices.

52. The method of item 51, wherein the data are sent, via the communication unit, to an external device.

53. The method of items 46-52, wherein one or more calibration plots are selected to analyze the electrode data.

54. The method of items 46-53, wherein a signal calibration is performed.

55. The method of item 54, wherein the calibration comprises at least one of a pH-based signal correction, a temperature-based signal correction, a humidity-based signal correction, a sensor bending signal calibration, a pressure-based signal calibration, or a signal calibration based on the signal of an analyte, or a combination thereof.

56. The method of items 46-55, wherein the array of sensors is attached to a leaf of the plant.

57. The method of items 46-56, wherein the array of sensors is attached to a stem of the plant.

58. The method of items 46-57, wherein the array of sensors is attached to at least two locations of the same plant.

59. The method of items 46-58, further comprising measuring kinetics of the one or more phytohormones in the plant.

60. The method of items 46-59, further comprising measuring the distribution the one or more phytohormones in the plant.

61. The method of items 46-60, further comprising continuously measuring one or more conditions of one plant or a plurality of plants.

62. The method of items 46-61, further comprising modifying an irrigation control system in response to the conditions of a plant or a plurality of plants.

63. The method of items 46-62, further comprising modifying and/or applying a pesticide treatment in response to the conditions of a plant or a plurality of plants.

64. The method of items 46-63, further comprising modifying and/or applying a fertilizer or nutrient treatment in response to the conditions of a plant or a plurality of plants.

65. The method of items 46-64, further comprising harvesting the plant in response to the conditions of a plant or a plurality of plants.

66. The method of items 46-65, further comprising continuously measuring a second parameter selected from a physical parameter or a chemical parameter in response to the conditions of a plant or a plurality of plants.

67. The method of items 46-66, wherein the second parameter comprises humidity, temperature, soil conditions, plant growth, pH, or volatile organic compounds.

68. The method of items 46-67, wherein the one or more conditions are detected with a deviation of less than 10%, or less than 5%, or less than 1%, or less than 0.5%, or less than 0.1%, wherein the deviation is across at least three repeated measurements.

69. A method of manufacturing a flexible plant sensor, comprising:
a) attaching a transfer film onto a part of a flexible substrate;
b) forming a pattern in the transfer film attached to the flexible substrate, wherein the pattern is not continuously present in the flexible substrate;
c) removing a portion of the transfer film on the flexible substrate corresponding to the formed pattern to expose a portion of the flexible substrate;
d) applying an ink solution uniformly over the exposed portion of the flexible substrate;
e) curing the dispersion solution at 50° C. to 100° C. for 5 minutes to 60 minutes; and
f) removing the remaining transfer film from the flexible substrate to obtain the flexible plant sensor.

70. The method of item 69, further comprising forming one or more additional patterns in the transfer film by repeating steps b to e prior to step f.

71. The method of any one of items 69-71, wherein the flexible substrate is a flexible thermoplastic and/or thermosetting film.

72. The method of any one of items 69-71, wherein the flexible substrate is a flexible polyimide film or a perfluorinated sulfonic-acid isomer based film.

73. The method of any one of items 69-72, wherein the flexible substrate is 50 μm to 500 μm thick.

74. The method of any one of items 69-73, wherein the step of forming the pattern comprises cutting the transfer film at a speed of 50 mm/s to 200 mm/s, or 75 mm/s to 150 mm/s, or 80 mm/s to 100 mm/s, or 90 mm/s, or 91 mm/s, or 92 mm/s, or 93 mm/s, or 94 mm/s, or 95 mm/s, or 96 mm/s, or 97 mm/s, or 98 mm/s, or 99 mm/s.

75. The flexible plant sensor of any one of items 69-74, wherein the step of forming the pattern comprises cutting the transfer film using a force of 1.00 N to 6.00 N, or 1.50 N to 5.50 N, or 2.00 N to 5.00 N, or 2.50 N to 4.50 N, or 3.0 N to 4.25 N, or 4.01 N to 4.10 N.

76. The method of any one of items 69-75, wherein the ink solution comprises at least one of a sol, a gel, or a paste.

77. The method of any one of items 76, wherein the ink solution comprises a N-Methyl-2-pyrrolidone (NMP), dimethylformamide (DMF), or deionized water.

78. The method of any one of items 69-77, wherein the ink solution comprises a printable conductive ink.

79. The method of any one of items 69-78, wherein the printable conductive ink is selected from graphene, reduced graphene oxide, Ag/AgCl, or a combination thereof.

80. The method of any one of items 69-79, further comprising drop coating one or more additional coatings on the flexible plant sensor.

81. The method of any one of items 69-80, wherein a second sensor is formed on the opposite side of the flexible substrate containing a first sensor.

82. The method of any one of items 69-81, wherein a first flexible substrate containing a first set of flexible plant sensors may be attached to a second flexible substrate containing a second set of flexible plant sensors using an adhesive.

### EXAMPLES

The following examples are intended to exemplify the present disclosures and are not limitations of the claimed invention. All molecules, compositions, methods, assays, and results disclosed in the examples form part of the present invention.

### Example 1

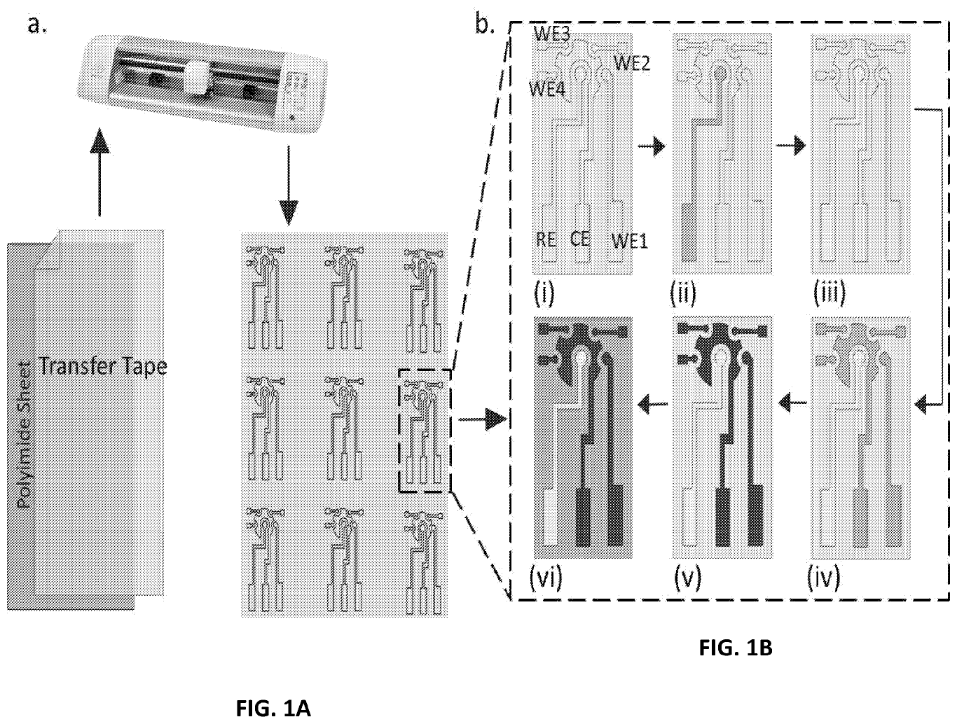

Screen Printed Electrode (SPE) Fabrication. A multiplexed electrochemical sensor was fabricated on a flexible polyimide sheet. The sensor was comprised of four working electrodes (WEs) for monitoring four phytohormones, one reference electrode (RE), and one counter electrode (CE). Firstly, the CAD design of the electrodes was loaded on a craft cutter printer. Next, a sheet of transfer tape with adhesive backing was attached to a 125 m thick polyimide sheet. The taped polyimide sheet was then inserted into the craft cutting machine, which cut an array of the electrode design on the transfer tape, as is illustrated in FIG. 1A. The cutter speed and force were adjusted to be 93 mm/s and 4 N, respectively so that only the transfer tape on top was cut without cutting the polyimide sheet underneath. After the electrode patterns were cut on the transfer tape (i) in FIG. 1B, the pattern of the RE region was removed, exposing the polyimide sheet in the RE region (ii) in FIG. 1B. Ag/AgCl paste was applied to this exposed region of the polyimide sheet using a small paintbrush (iii) in FIG. 1B. The sheet was then placed in an oven at 80° C. for 15 minutes for curing the Ag/AgCl ink. After that, the remaining cut parts of the transfer tape were removed (iv) in FIG. 1B and graphene ink was applied on top of the sheet and screen-printed using a squeegee (v) in FIG. 1B. The graphene ink was then dried in the oven at 300° C. for 15 minutes. After cooling the taped polyimide sheet to room temperature, the remaining transfer tape was peeled off (vi) in FIG. 1B. The final device had a length of 3.5 cm and a width of 2.5 cm. The diameter of each WE was 2 mm and the RE was 1 mm.

Formation of CuMOF-based Composite Coating. To selectively detect the SA, the graphene WEs were coated with a composite of copper-based metal-organic framework (CuMOF), which has unique characteristics such as a high surface area and tunable pore size. The carbon black (CB) functional material was incorporated into the CuMOF to enhance its hydrothermal stability.

The CuMOF coating was synthesized by dissolving 8 mL of N, Ndimethylformamide (DMF) with 8 mL of ethanol. Subsequently, 0.40 g of polyvinyl pyrrolidone (PVP) was added to the mixture and centrifuged at 200 rpm. Next, 46.64 mg of Cu(NO₃)₂·4H₂O and 10.86 mg of 2-amino terephthalic acid were dissolved in 4 mL of DMF and centrifuged to form a homogeneous mixture. Then this DMF solution was added to the PVP solution and ultrasonicated for 30 minutes. Afterward, the resulting solution was heated in an oven at 100° C. for 5 hours. The obtained precipitate was collected and dissolved in 40 mL of DMF, and heated at 100° C. for 8 hours. Subsequently, the CuMOF precipitates were obtained by centrifuging at 1000 rpm for 30 minutes. CuMOFs and CB (in a 2:1 ratio) were dissolved in 6 mL of deionized water and sonicated for 30 minutes. Next, 5 μL of nafion (w/v) (0.01%) was added to this solution and ultrasonicated for 30 minutes. As a result, a composite of CuMOFs-CB-nafion was obtained. 10 μL of the CuMOFs-CB-nafion solution was drop-coated on the WE.

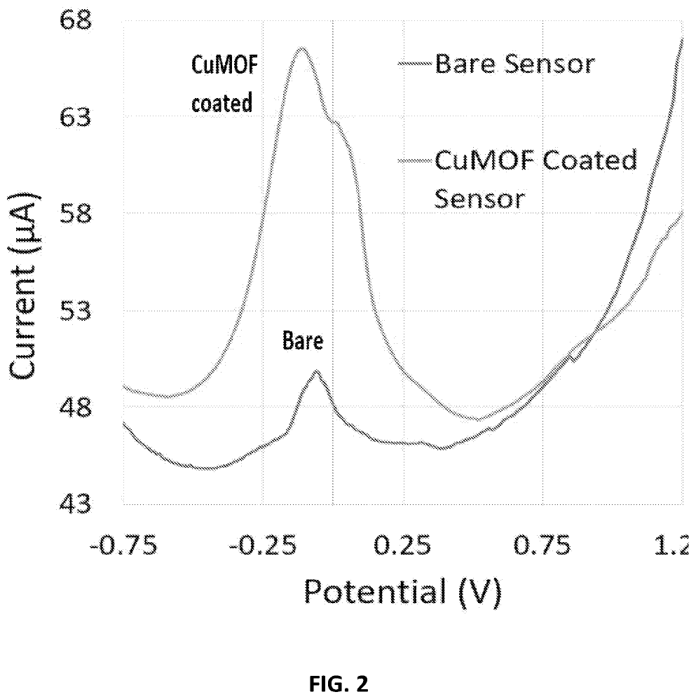

Electrochemical Characterization. A portable electrochemical workstation EmStat (PalmSense, Houten, Netherlands) was used to perform the electrochemical tests. Differential Pulse Voltammetry (DPV) was conducted in a voltage range from −1.0 V to 1.6V. All electrochemical measurements were performed in the 0.05 M tris-HCl (pH=7.1) buffer. FIG. 2 shows DPV responses for the bare electrode and CuMOF-coated electrode. The CuMOF coating introduced a new redox current peak at −0.17 V, which was due to the presence of the copper ions in the electrode, indicating the ability of CuMOF to promote the oxidation of SA. The bare electrode peak shifted from −0.06 V to 0.009 V.

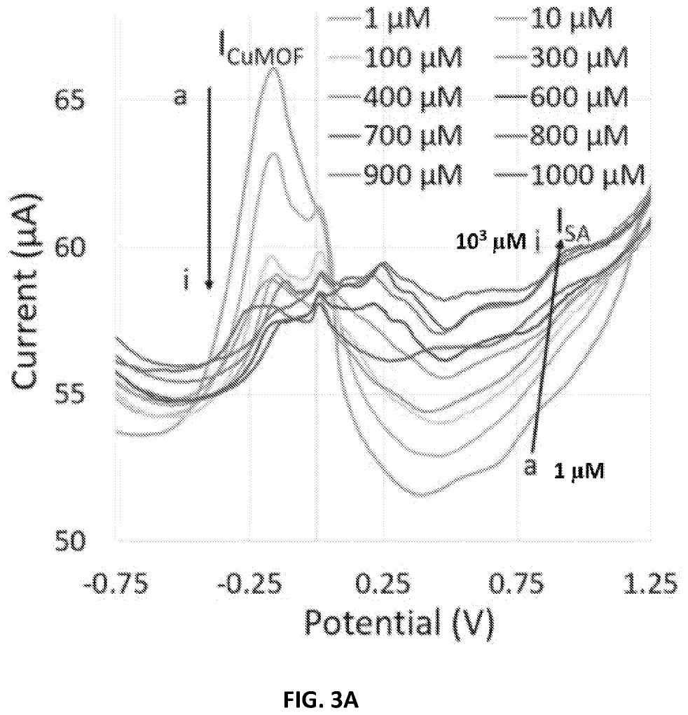

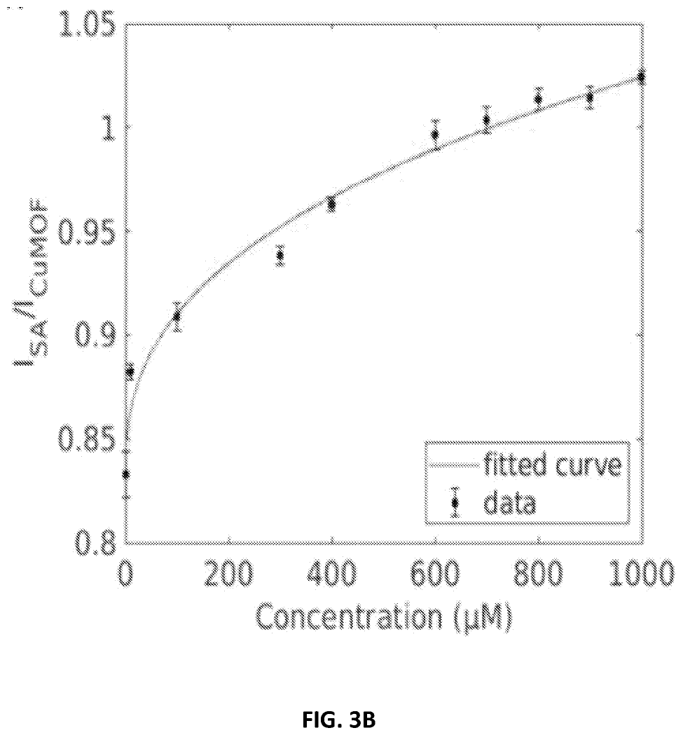

Electrochemical Measurements of SA. The electrochemical sensor was designed to quantitatively measure four phytohormones on a single chip or patch. FIG. 3A shows the DPV responses of the sensor for SA concentrations of 1, 10, 100, 300, 400, 600, 700, 800, 900, and 1000 μM. FIG. 3A shows a redox peak at around −0.17 V (the CuMOF peak), which was due to the reduction of Cu²⁺ in the CuMOF. Another redox peak at ˜0.85 V (the SA peak) accounted for the SA oxidation. As the SA concentration increased, the reduction peak current for CuMOF decreased, indicating that CuMOFs effectively catalyzed the oxidation of SA. On the other hand, the oxidation peak current for SA increased with increasing concentrations of SA. The CuMOF and SA redox peaks were separated by 1.02 V and hence the ratio of the two peak currents (ISA/ICuMOF) was used as the response signal for the sensor. FIG. 3B shows the calibration plot of ISA/ICuMOF as a function of SA concentrations. The fitted curve is given by Formula (1) having R²=0.98:

$${I}_{S A} / {I}_{CuMOF} = 0.01332 {C}^{0.3872} + 0.8 \; 309 \qquad ( 1 )$$

where, C denotes the concentration in M. Following the sensitivity calculations, the sensitivity at lower concentration of 1 μM was found to be 0.0051 μM⁻¹ and at higher concentration of 900 μM was found to be 0.000078 μM⁻¹. The limit of detection was found to be 11.3 μM.

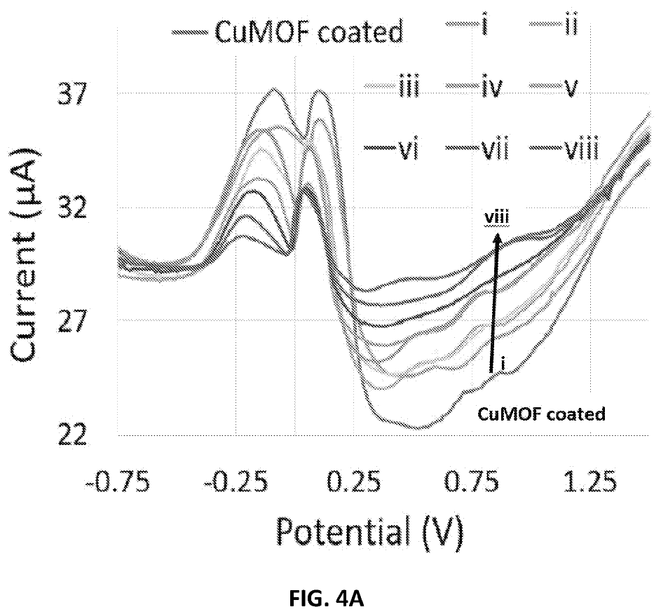

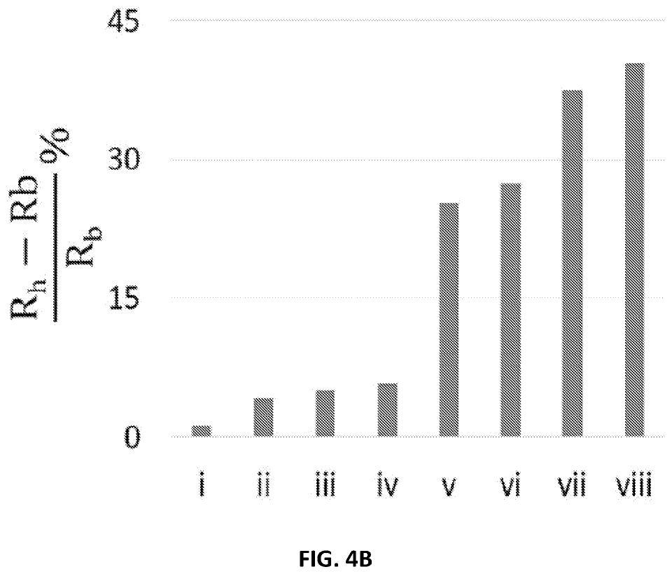

Selectivity test. Plant sap contains several interfering species including Jasmonic acid (JA), Indole-3-acetic acid (IAA), and glucose. Hence, we investigated the specificity of our sensor to SA in the presence of these interfering species as is illustrated in FIGS. 4A-4B. The sensor was exposed to the following solutions:
i. 50 μM of JA
ii. 50 μM of IAA
iii. 50 μM of glucose
iv. a mixture of 50 μM of JA, 50 μM of IAA, and 50 μM of glucose
v. 100 μM of SA
vi. a mixture of 100 μM SA, 50 μM of JA, 50 μM of IAA, and 50 μM of glucose
vii. 900 μM of SA
viii. a mixture of 900 μM of SA, 50 μM of JA, 50 μM of IAA, and 50 μM of glucose

The relative signal was defined by the following Formula (2):

$$R elative Signal = ( {R}_{h} - {R}_{b} ) / {R}_{b} \qquad ( 2 )$$

where Rh represents the ratio of the hormone redox current to the CuMOF redox current and Rb represents the ratio of the base current to the CuMOF redox current when no hormones were present in the solution. As was evident from FIG. 4B, the relative signal was much higher for SA as compared to that with the interfering species.

### Example 2

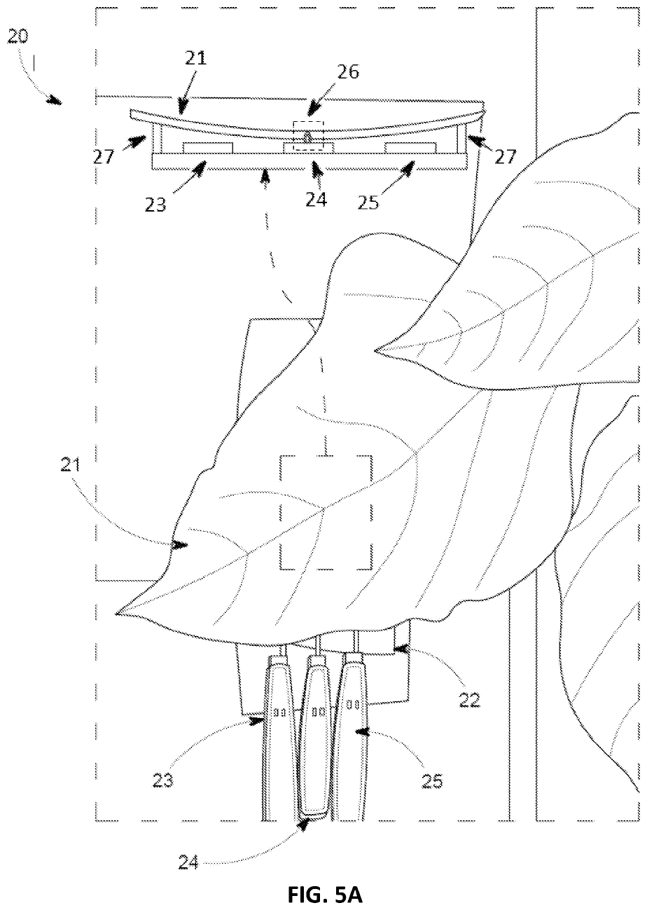

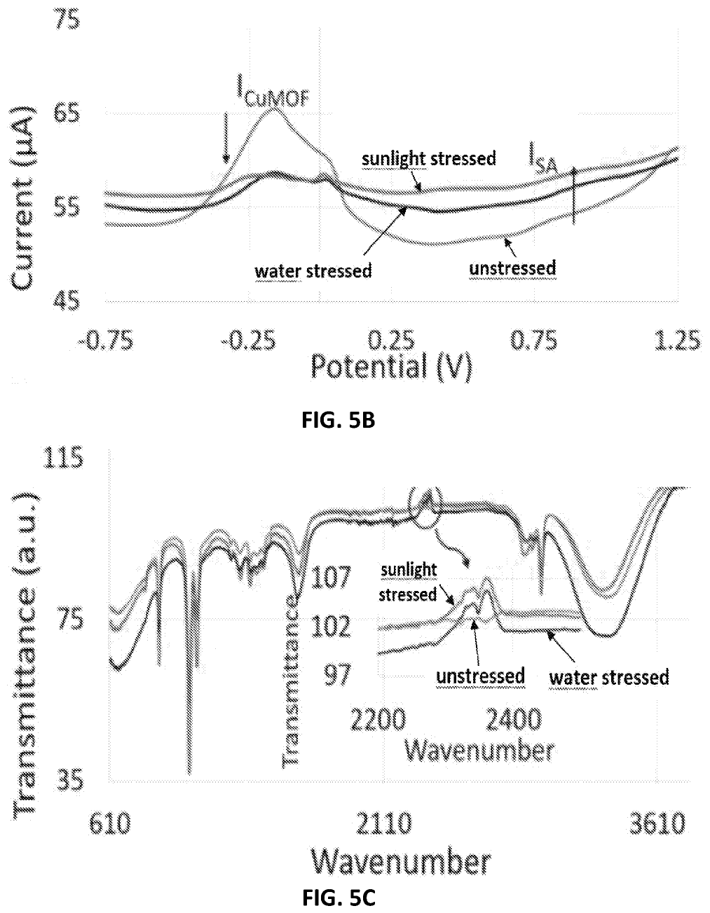

SA Detection in Living Plants. For in-situ experiments, the screen-printed flexible sensor of Example 1 was installed on the leaves of a red bell pepper plant. FIG. 5A shows a schematic representation of the flexible plant sensor 20 on the leaf 21. An inset box on the leaf 21 illustrates the configuration of the sensor on the side of the leaf facing away from the presented view point. The plant leaf 21 is penetrated by a tiny hole 26 (approximately 10 μm) that was punched on the vascular bundle of the leaf and the sensor 20 was placed beneath the leaf. To collect enough sap (˜20 μL) for the biosensor to detect the SA concentrations, a spacer (inset of FIG. 5A) was fabricated by cutting a 1.5 cm diameter hole on a separate polyimide sheet. This polyimide spacer 27 (125 μm thick) was adhered to the sensor in such a way that only the detection region of the electrodes was exposed to the leaf while covering the conductive traces. The sensor 20 comprising a flexible substrate 22, a reference electrode 23, a counter electrode 24, and a working electrode 25, together with the spacer 27, was fixed on the back of the leaf (FIG. 5A). FIG. 5B shows the DPV results for the unstressed (control), water-stressed, and sunlight-stressed plants. The water and sunlight-stressed plants did not get water and sunlight respectively, for four days. It was evident that the unstressed plant had the lowest I_{SA}/I_{CuMOF} ratio (0.9042), while the sunlight stressed plant showed the highest I_{SA}/I_{CuMOF} ratio (0.94319). The sensor calibration plot as shown in FIG. 3B was used to estimate the concentrations of salicylic acid in the leaves of unstressed, water- and sunlight-stressed plants. The results are listed in Table I.

TABLE I

| Plant | FTIR | Electrochemical | Relative |
|---|---|---|---|
| Stress | Reference | Sensor | Deviation |
| Condition | (μM) | (μM) | (%) |
| Unstressed | 84.52 | 81.79 | 3.33 |
| Water Stressed | 166.08 | 162.41 | 2.26 |
| Sunlight Stressed | 241.92 | 246.098 | 1.69 |

Next, the SA levels measured by our sensor were validated against the values from Fourier Transform Infrared Spectroscopy (FTIR) tests conducted on the sap collected from the leaves. FIG. SC shows the FTIR responses for sap solutions collected from unstressed, water-stressed, and sunlight-stressed plants. A similar trend was observed with our sensor, thereby confirming its reliability. The comparative analysis of SA levels measured with our sensor and the FTIR test is shown in Table I.

### Example 3

### Screen Printed Interdigitated Electrodes (IDEs) on Polyimide

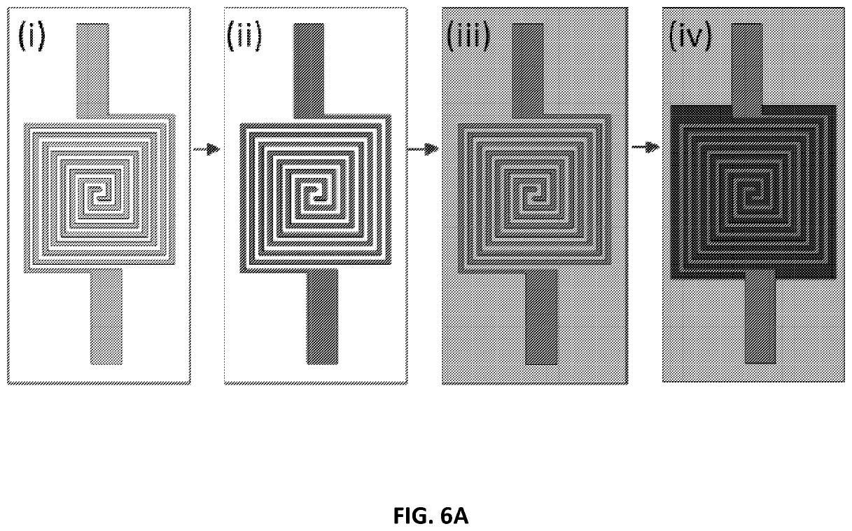

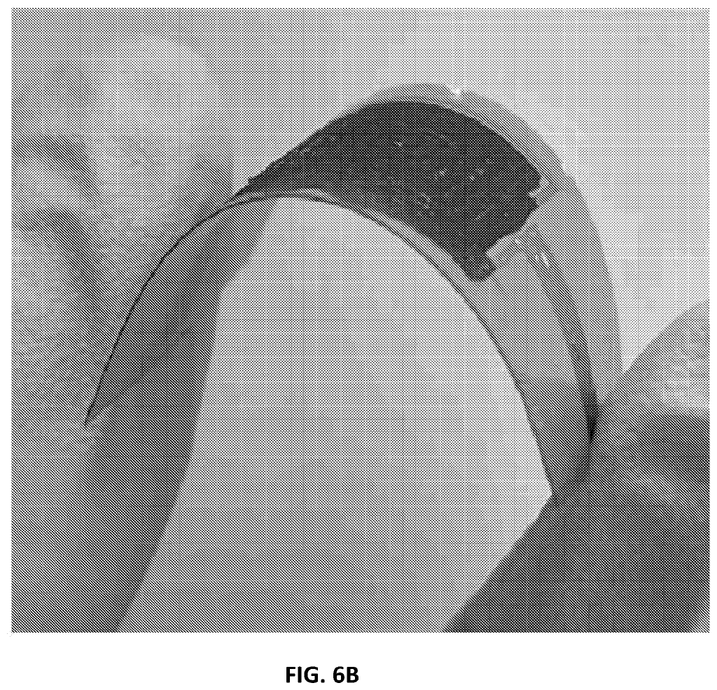

FIG. 6A schematically shows the fabrication of the flexible and bendable strain sensor using a simple and cost-effective screen-printing procedure. The design of the IDEs was generated using AutoCAD Fusion 360, followed by loading the design on a craft cutter. A transfer tape was attached to a 125 μm thick polyimide sheet, which was then inserted into the craft cutter. The cutter speed was 100 mm/s, and the force was 4 N. These cutter parameters were chosen to cut only the transfer tape at the top, but not the polyimide sheet. After the electrode pattern was cut on the transfer tape atop the polyimide sheet, the patterns were removed using a tweezer (i) in FIG. 6A, and graphene ink was applied over the exposed polyimide patterns (ii) in FIG. 6A. A squeegee was used to uniformly spread the graphene ink, which was thermally cured at 100° C. for 60 minutes. Finally, the remaining transfer tape was removed to achieve graphene IDEs on polyimide with an overall dimension of 2 cm×2 cm (iii) in FIG. 6A. The IDE traces were 600 μm thick with an electrode spacing of 600 μm.

Reduced Graphene Oxide (rGO) Dispersion Preparation. A thin layer of rGO was drop-coated on the graphene IDEs (iv) in FIG. 6A. In this step, a 2 mg/mL rGO dispersion solution was prepared in N-Methyl-2-pyrrolidone (NMP) through ultrasonication for 1 hour. 90 μL of this resulting dispersion solution was drop cast on the graphene IDEs. The device was then heated on a hotplate at 80° C. for 10 minutes. After the NMP evaporated, the graphene IDEs were coated with a thin layer of rGO, thereby resulting in a flexible strain sensor, as is shown in FIG. 6B.

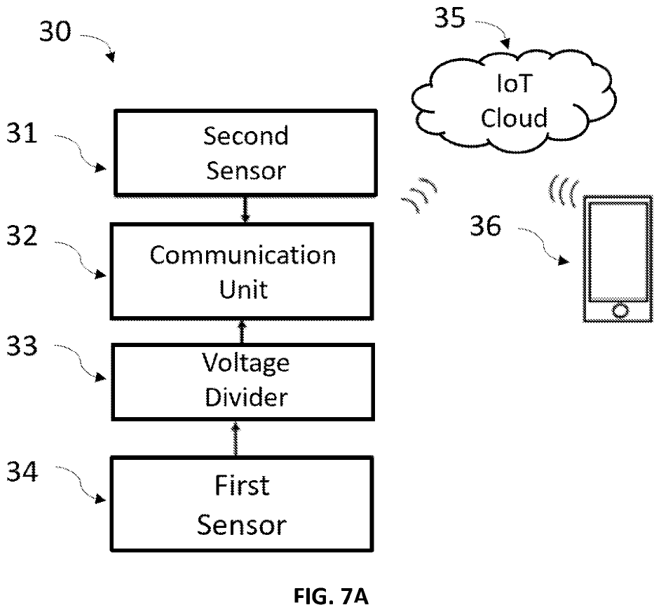

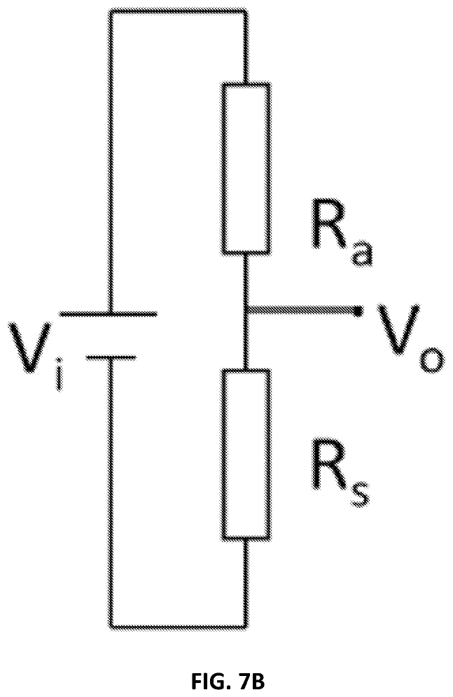

Resistance Measurements. For data acquisition, the strain sensor of Example 3 was interfaced with an Arduino MKR1000 Wi-Fi board which had a 32-bit low-power microcontroller unit. The auto ranging functionality of the MKR1000 board was used to measure the resistance variations of the strain sensor in response to varying degrees of bending. Cylindrical blocks of varying diameters were made by a 3D printer to mimic the stems of small plants such as bell pepper (stem diameter=0.6 cm), cucumber (stem diameter=1.1 cm), squash (stem diameter=1.3 cm), tomato (stem diameter=1.34 cm), and maize (stem diameter=2.8 cm) plants. The strain sensor was wrapped around the blocks, and the resistance variations resulting from varying degrees of bending were measured via the Arduino board. The board had a built-in Wi-Fi module, which transmitted the data wirelessly to an IoT platform called Blynk cloud and then to the Blynk mobile application. The system was controlled remotely via the Blynk application on a smartphone. The overall system is similar to the exemplary architecture that is shown in FIG. 7A. The system 30 includes first and second sensors (31, 34), operatively connected to a communication unit 32 and a voltage divider 33. The communication unit 32 is configured to transmit information/data to an IoT cloud system 35 that can accessed through an external device 36. The mobile application displayed the diameter of the crop stem (mimicked through the 3D printed cylindrical blocks), real-time resistance variations of the developed strain sensor, and temperature of the ambient medium, as described below.

A voltage divider circuit was designed to convert the resistance variations of the strain sensor into voltage signals that were detected by the microcontroller. FIG. 7B shows the voltage divider circuit where Ra represents a known resistance, with respect to which the unknown resistance, Rs, of the strain sensor was measured. Rs varied from 200 kΩ to 10 MΩ. Hence, Ra values were selected to be 250k, 330k, 680k, 820k, 1 M, and 4.7 M. The microcontroller automatically selected the Ra values based on the detected Rs. Vi was the same as the supply voltage (=3.3V) for the Arduino board, and V₀ was the measured voltage across the strain sensor. The unknown resistance, Rs, of the strain sensor, was measured using the voltage divider formula (3):

$${R}_{s} = ( {R}_{a} ) / ( ( {V}_{i} / {V}_{0} ) - 1 ) \qquad ( 3 )$$

Temperature Measurements. In an actual agricultural field setting, ambient temperature is expected to affect the resistance of the strain sensor. To correct for temperature effects, the strain sensor was calibrated for different temperatures ranging from 25° C. to 45° C. To measure the temperature, the TMP 36 sensor was used which could detect temperatures ranging from −45° C. to 125° C. The temperature measured by TMP 36 is expected to be more representative of the canopy temperature.

Interfacing with Arduino. The MKR1000 Arduino board was programmed with the Arduino Integrated Development Environment. The Arduino board was programmed to first read the ambient temperatures detected by the TMP 36 sensor, and then select the stored calibration curve of the strain sensor based on the detected temperature. The board was connected to the Wi-Fi network to transmit the real-time temperature and resistance measurements to the Blynk cloud platform.

Strain Measurements. The strain sensor was wrapped around various uniform cylindrical blocks with the following diameters: 0.79 cm, 0.916 cm, 1.06 cm, 1.09 cm, 1.27 cm, 1.43 cm, 1.76 cm, 2.18 cm, 2.5 cm, 2.85 cm, and 3.6 cm. The blocks were designed in AutoCAD Fusion 360 and printed using the Form 3B 3D printer. The degree of curvature of the strain sensor is given by the following Formula (4):

$$θ = \frac{3 ó 0 S}{2 π r} \qquad ( 4 )$$

where θ is the angle of curvature, S is the arc length and r is the radius of curvature. The length of our strain sensor was 2 cm, hence S=2 cm and r is the radius of the cylinders. The response of the strain sensor was defined as ΔR/R₀, where R₀ was the initial value of resistance in the absence of any bending, and ΔR=Rₛ−R₀ (with Rₛ shown in FIG. 7B) was the resistance variations under an applied strain.

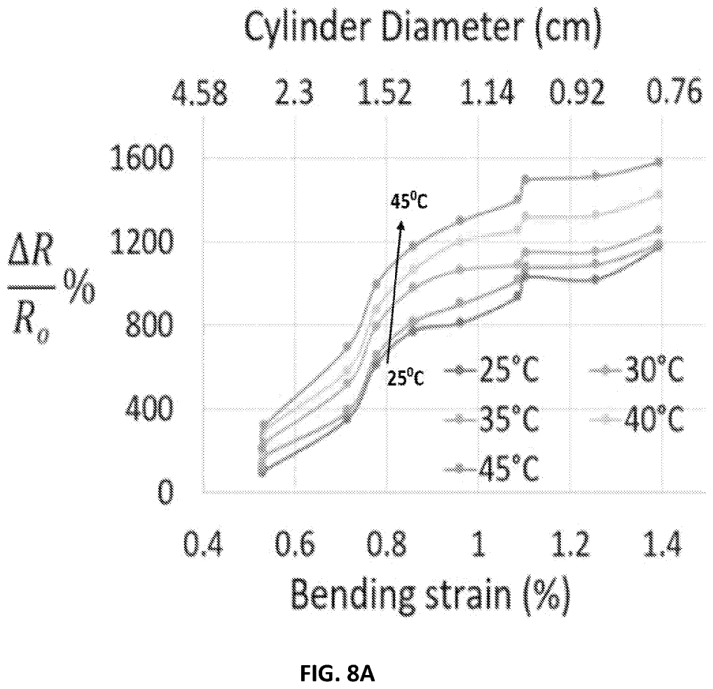

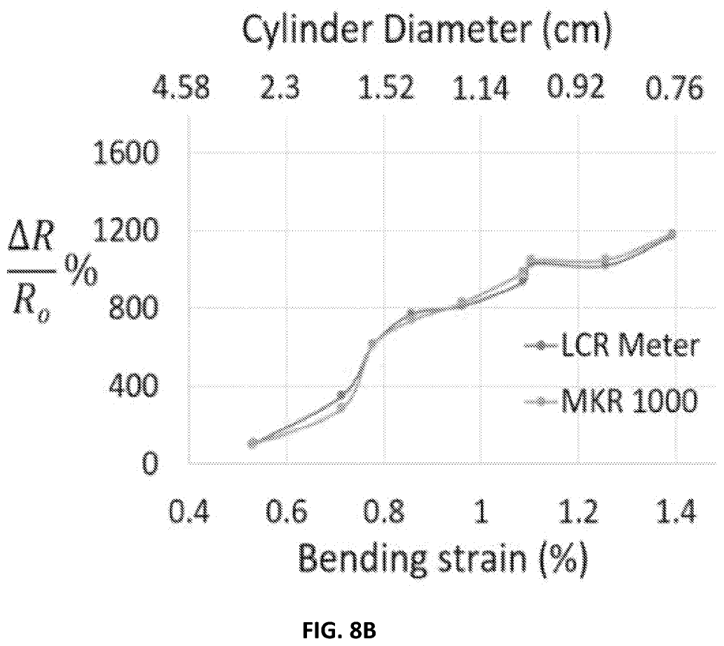

The responses of the strain sensor were measured with the Applent AT3817 LCR meter. In the absence of any bending, the resistance of the sensor was R₀. FIG. 8A shows the response of the strain sensor as a function of bending strain and stem diameters (mimicked through the cylinders) at five different temperatures. As can be seen from FIG. 8A, the resistance increased with an increased bending strain and decreased cylinder diameter. In addition, the curve shifted toward increased resistance as the temperature increased as was also observed in. The gauge factor at a strain of 1.39% was found to be 827. Table II shows performance comparison of our strain sensor with current thin-film sensors and our sensor is superior in terms of gauge factor and remote monitoring capability. The smallest angle of curvature detected by our sensor was 0.0180.

TABLE II

| | Sensor | Gauge | Strain | |
|---|---|---|---|---|
| | Material | Factor | (%) | Connectivity |
| Comparative | Ag | 39 | 0.26-0.62 | N/A |
| Example | nanowire | | | |
| Comparative | Graphene platelets | 181 | 0-7.5 | N/A |
| Example | and multi-walled | | | |
| | CNT on PU | | | |
| Comparative | rGO on | 70 | 0-6 | N/A |
| Example | paper | | | |
| Working | rGO on | 827 | 0.53-1.39 | Wi-Fi |
| Example | polyimide | | | |

The response of the strain sensor was measured with the MKR1000 Arduino board and compared with the measurements recorded by the LCR meter. FIG. 8B shows the sensor responses at 25° C. measured with the LCR meter and the MKR1000 board. The mean deviation between the two measurements was calculated to be only 3.5%.

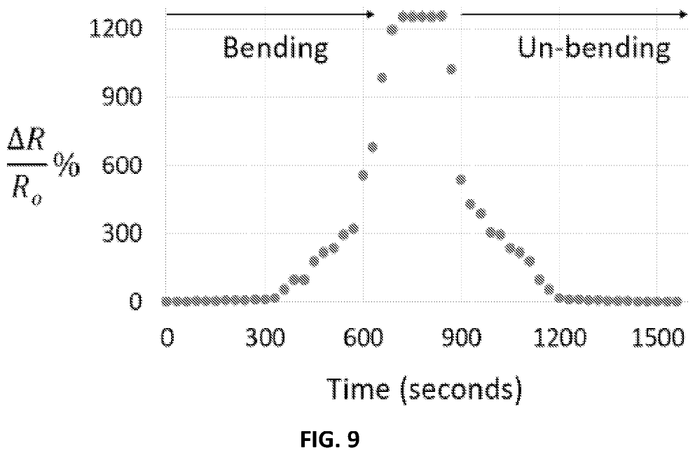

Repeatability Studies. The response of the strain sensor was further shown under bending and unbending cycles. FIG. 9 shows the sensor response as a function of time, measured with the MKR1000 board at 25° C. During the bending cycle, the sensor was wrapped around a 0.916 cm diameter cylinder, which put tension on the sensor and increased its resistance. During the unbending cycle, the sensor returned to the relaxed state and its resistance reduced to the baseline value. These results demonstrated the repeatable behavior of our strain sensor.

### Example 4

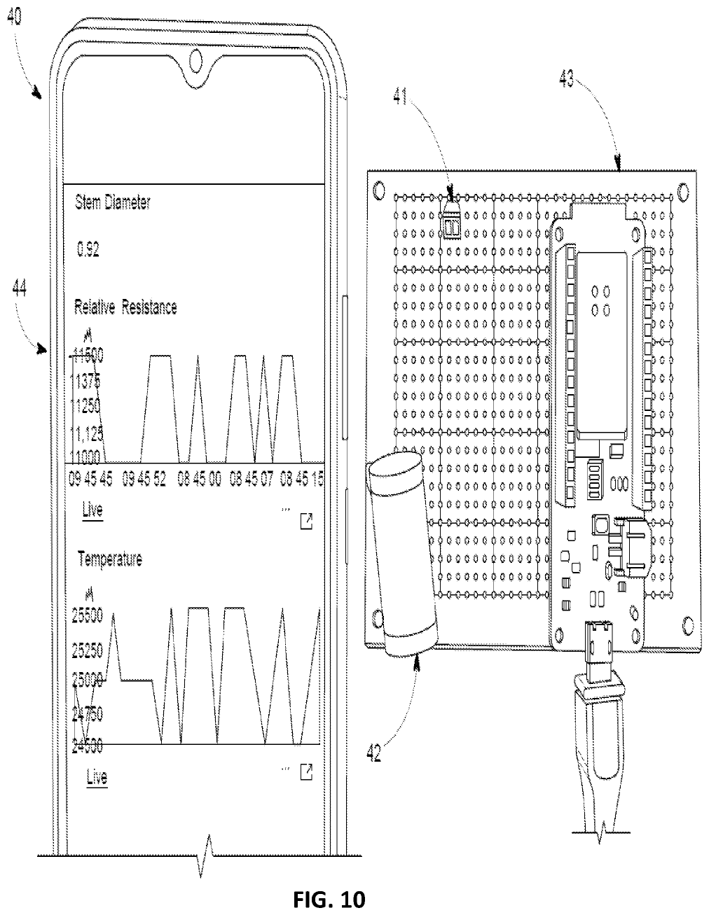

Real-Time Bending Measurements with IoT. FIG. 10 illustrates an exemplary stem diameter monitoring system 40 where data was accessed through an external device 44 (e.g., a smartphone application). The strain sensor 42 as described in Example 3 was mounted on a cylinder block and connected to a voltage divider (not shown), the analog output of which was detected by the data acquisition system 43 (the onboard SAMD21 Cortex-MO microcontroller) and converted to a digital signal. The microcontroller processed the readings from the temperature and strain sensors (41, 42) to select a previously stored calibration curve (that is shown in FIG. 8A). The diameter of the cylinder was then estimated from the calibration curve and the microcontroller transmitted this information to a peripheral smartphone using the WINC1500 Wi-Fi module. The Blynk mobile application displayed the diameter of the cylindrical block (mimicked as the stem diameter), the real-time response (i.e., relative resistance, ΔR/R₀) of the strain sensor, and the ambient temperature. A p-value of 0.0012 indicated that the cylinder diameters estimated by our sensor system were significantly close to the actual diameters.

### Example 5

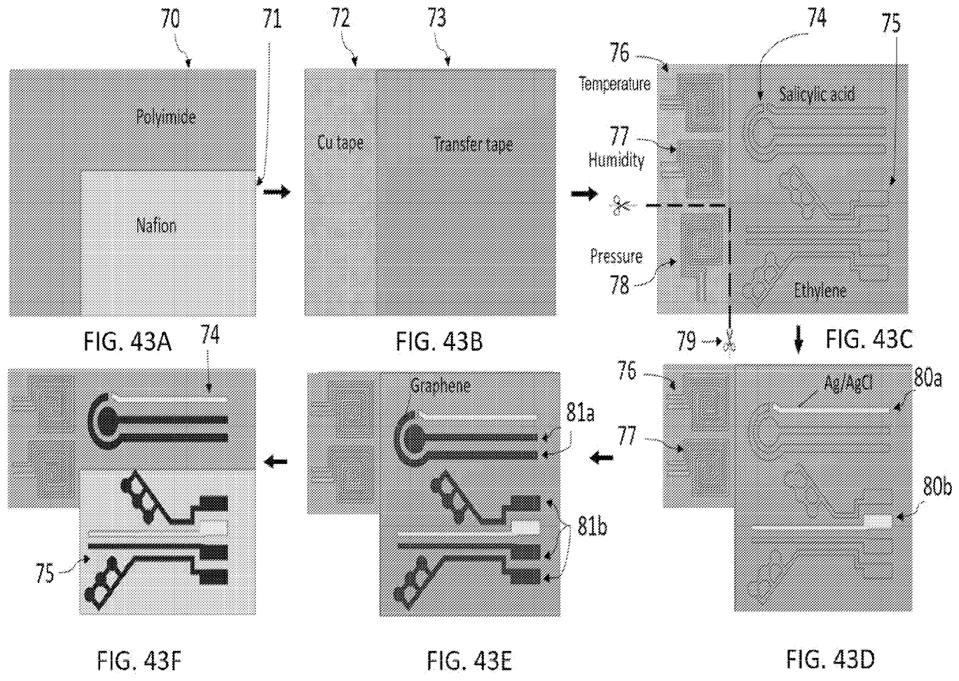

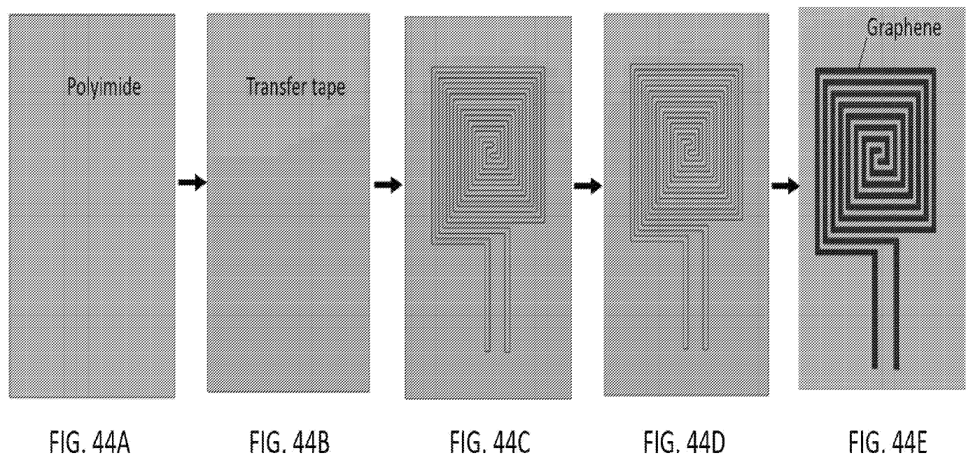

Sensor Fabrication. The hormone sensors (salicylic acid and ethylene) were comprised of three electrodes: working electrode, counter electrode, and reference electrode. The temperature, humidity, strain, and pressure sensors, each composed of two electrodes. The conductive electrodes for salicylic acid, temperature, humidity, strain, and pressure sensors were fabricated on a flexible polyimide substrate, while the ethylene sensor was made on a flexible Nafion sheet using a screen-printing technique. The thicknesses of the polyimide and Nafion sheets were 125 μm and 150 μm, respectively. FIGS. 43A-43F illustrate the step-by-step fabrication procedure for the salicylic acid sensor 74, ethylene sensor 75, temperature sensor 76, humidity sensor 77, and pressure sensor 78 and FIGS. 44A-44E show the fabrication procedure for strain sensor. First, the 2D design of the sensors was developed using AutoCAD Fusion 360 and the design was exported to the PrismCut craft cutter. A single piece of polyimide sheet 70 was used to fabricate all of the sensors, to achieve roll-to-roll production. One-third of the polyimide sheet 70 was taped with a Nafion sheet 71 at one corner for screen printing the ethylene sensor 75 (FIG. 43A). A copper tape 72 was attached to the areas dedicated for temperature, humidity, and pressure sensors, while a transfer tape 73 was attached to the areas meant for salicylic acid and ethylene sensors (FIG. 43B). Subsequently, the polyimide sheet was loaded to the craft cutter. The PrismCut machine cut the electrode patterns using a blade with 30° angled tip at a speed of 97 mm/s and a force of 4.01 N for salicylic acid 74 and ethylene sensors 75, and at 80 mm/s speed and 3.57 N force for temperature 76, humidity 77, and pressure sensors 78 (FIG. 43C). Next, the pressure sensor was cut off the polyimide sheet (as shown by cut lines 79), the undesired portions of the Cu tape were peeled off with a tweezer to produce the copper interdigitated electrodes (FIG. 43D).

The next steps involved screen printing of conductive inks to produce the electrodes. First, the transfer tape was removed from the areas meant for the reference electrodes of the salicylic acid 74 and ethylene sensors 75. A squeegee was used to apply a uniform layer of Ag/AgCl paste 80a and 80b on the exposed regions (FIG. 43D). Next, the polyimide sheet was placed inside a convection oven and heated at 80° C. for 30 minutes. After the annealing of the Ag/AgCl paste, the sheet was removed from the oven and cooled down to the room temperature. Similarly, the working and counter electrodes of the salicylic acid and ethylene sensors were screen printed with graphene ink (81a, 81b; FIG. 43E). The graphene ink was heated in the oven for 30 minutes at 100° C. Finally, the transfer tape was peeled off to transfer the electrode patterns to the polyimide sheet (FIG. 43F).

It is to be noted that the strain and pressure sensors were made on the opposite sides of the same piece of polyimide sheet. FIGS. 44A-44E illustrate the step-by-step fabrication of the strain sensor. The process is similar to the screen printing of salicylic acid and ethylene sensors, i.e., attaching a transfer tape (FIG. 44B) to a bare polyimide sheet shown in FIG. 44A, cutting the interdigitated electrode patterns on the transfer tape (FIG. 44C), peeling off the transfer tape from the electrode patterns (FIG. 44D), and coating the exposed areas with graphene ink and subsequently curing the ink (FIG. 44E).

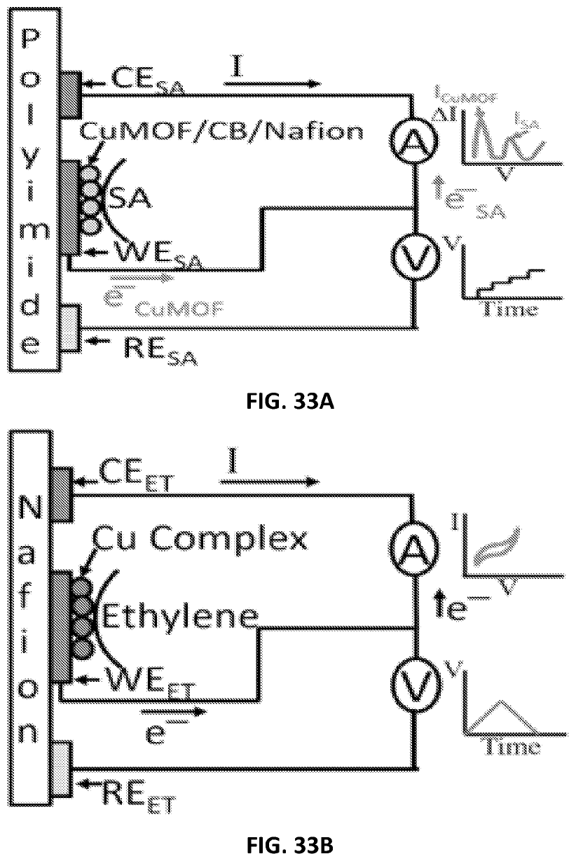

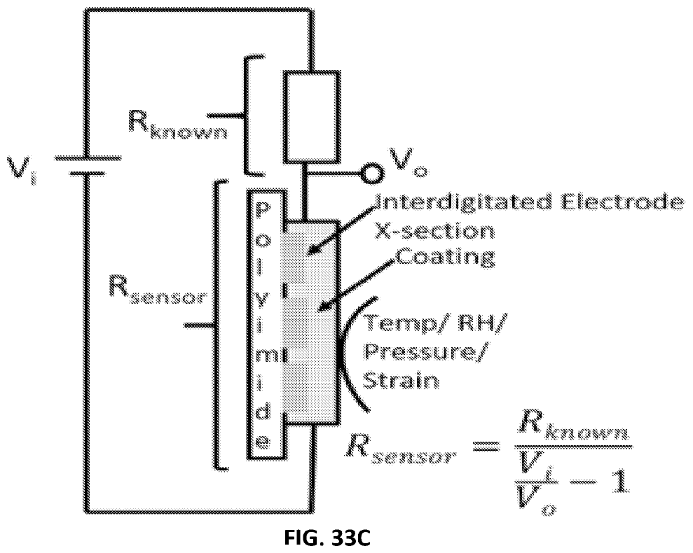

Salicylic Acid sensor. The sensing mechanism relied on electrochemistry wherein the redox reaction of SA on the chemically functionalized working electrode (WE_{SA}) was translated to a current flow proportional to the hormone concentration. The sensing mechanism of the electrochemical SA sensor is illustrated in FIG. 33A. The working electrode was coated with a CuMOF:CB: Nafion layer selective to SA as explained below. The data logger (commercial EmStat potentiostat) sent a staircase voltage pulse between the working (WE_{SA}) and reference electrodes (RE_{SA}) of the sensor. As a result, SA was oxidized on the working electrode and formed SA+, while the CuMOF was reduced. This redox reaction generated a current flow between the working (WE_{SA}) and counter (CE_{SA}) electrodes, recorded and analyzed by the data logger. A ratiometric approach was used to compute the response signal from the ratio of SA and CuMOF oxidation peak currents. The current ratio was correlated to the concentration of SA oxidized at the WE_{SA} surface.

Ethylene sensor. The ethylene sensor was made on a thin Nafion sheet. A triangular voltage waveform was applied between the working (WE_{ET}) and reference (RE_{ET}) electrodes by our data acquisition and processing (DAP) module. Upon exposure to gaseous ethylene, the active sites in the composite layer of copper complex and SWCNT were blocked by ethylene, which resulted in a decrease in the current measured between WE_{ET} and CE_{ET}. The Nafion sheet enabled electrolysis in the gas phase without the need for any buffer solution (FIG. 33B).

Temperature, humidity, pressure, and strain sensors. The temperature, humidity, pressure, and strain sensors were each made of two interdigitated electrodes. The dimension of the interdigitated electrode was the same for all the sensors. The impedance of the electrode was a function of electrode separation, electrode length, width, and the coating property. The resistance of the selective coating changed in response to variations in temperature/RH/pressure/strain (FIG. 33C). An Applent LCR meter (AT 3817A) was used to calibrate the sensors. However, to make the system wearable and collect in situ measurements from plants, the sensor was connected in series with a known resistor to form a voltage divider circuit, as also explained in the main article. As a result, the resistance variations were converted into voltage signals that were detected by a microcontroller. FIG. 33C shows the voltage divider circuit where R_{known} represents a known resistance and Rₛₑₙₛₒᵣ is the sensor resistance. Vᵢ represents the supply voltage of 3.3V and V₀ is the voltage measured across the temperature/RH/pressure/strain sensor. The auto-ranging functionality was adopted to select R_{known} from some previously known resistor values. The resistance of the sensor, Rₛₑₙₛₒᵣ, was measured using the following voltage divider formula: Rₛₑₙₛₒᵣ=(R_{known})/((Vᵢ/V₀)−1).

### Example 6

### Synthesis of Selective Sensor Coatings

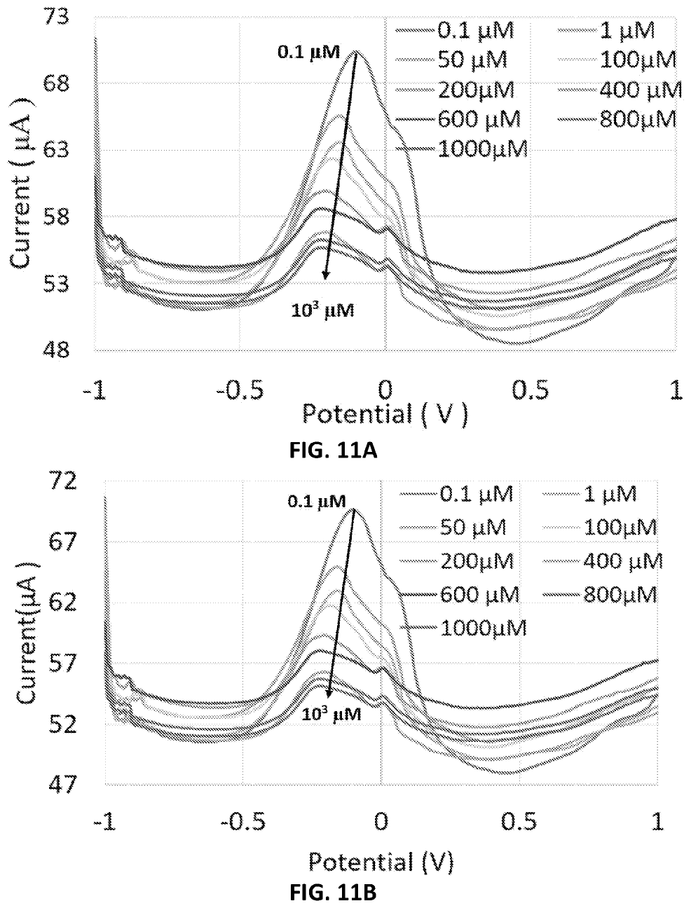

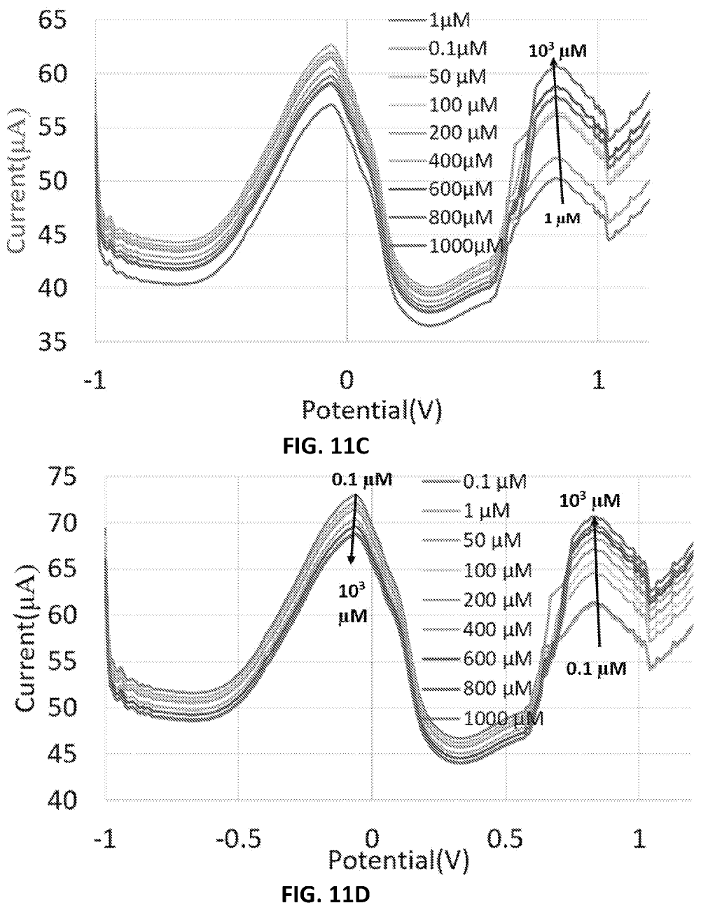

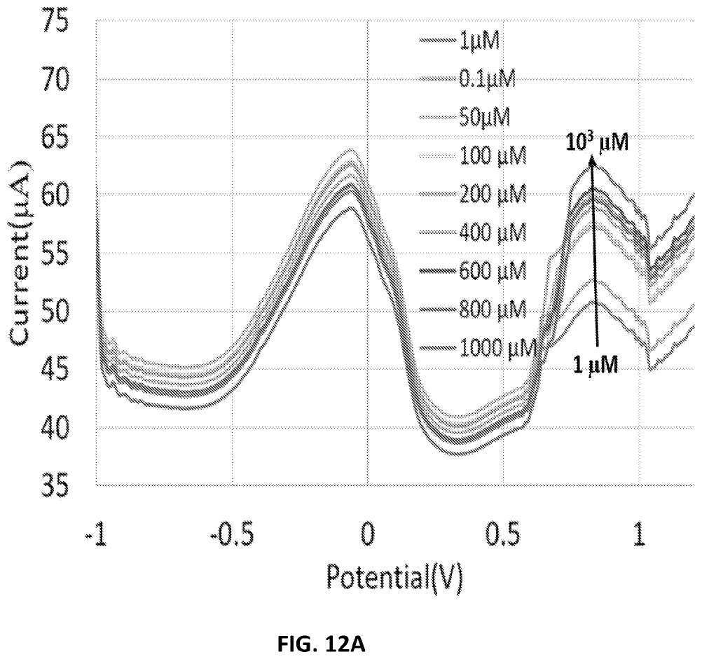

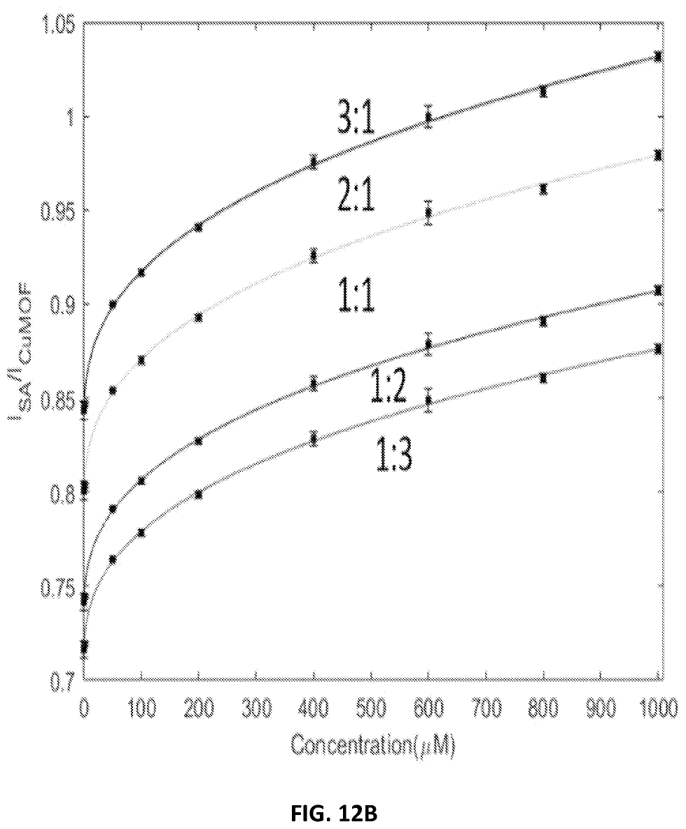

A composite coating of copper metal organic framework-carbon black-Nafion was prepared for Salicylic Acid sensing. The synthesis of copper-based metal organic framework (CuMOF) was prepared by mixing 8 mL of dimethylformamide (DMF) and 8 mL of ethanol and then centrifuging at 300 rpm for 15 minutes followed by ultrasonication for 30 minutes. Afterwards, 0.4 g of polyvinyl pyrrolidone (PVP) was added to the mixture and sonicated for 30 minutes. Next, 46.64 mg of copper (II) nitrate tetrahydrate was separately mixed with 10.86 mg of 2-aminoterephthalic acid in 4 mL of DMF and ultrasonicated for 30 minutes. The resulting solutions were mixed and ultrasonicated for 40 minutes. The mixture was then heated in a convection oven at 100° C. for 5 hours. The resulting green precipitate was collected and subsequently dissolved in 40 mL of DMF. Next, this solution was centrifuged at 1000 rpm for 30 minutes and a green CuMOF precipitated at the bottom. The supernatant DMF was removed and the CuMOF precipitate was dried at 50° C. in the oven. To obtain the optimized coating for SA, five different weight ratios (1:3, 1:2, 1:1, 2:1, 3:1) (w/w) of CuMOF and carbon black (CB) were dissolved in DI water by ultrasonication for 30 minutes. Next, 5 μL of nafion (w/v) (0.01%) was added to this solution and ultrasonicated for 30 minutes to obtain a composite of CuMOFs-CB-nafion. Finally, 10 μL of the composite solution was drop casted on the working electrode of the SA sensor. The 2:1 weight ratio of CuMOF and CB provided the highest sensitivity and peak current in response to SA level variations, as demonstrated in FIGS. 11A-11D, FIGS. 12A-12B, and Table III.

TABLE III

| | Low Conc. | High Conc. | | Limit of | |
|---|---|---|---|---|---|
| CuMOF:CB | Sensitivity | Sensitivity | | Detection (LOD) | |
| 1:3 | 0.002216 μM⁻¹ | 7.32589 × 10⁻⁵ | μM⁻¹ | 0.6977 | μM |
| 1:2 | 0.002115 μM⁻¹ | 7.3158 × 10⁻⁵ | μM⁻¹ | 0.678 | μM |
| 1:1 | 0.002233 μM⁻¹ | 7.3894 × 10⁻⁵ | μM⁻¹ | 0 . . . 687 | μM |
| 2:1 | 0.002264 μM⁻¹ | 7.409 × 10⁻⁵ | μM⁻¹ | 0.664 | μM |
| 3:1 | 0.002198 μM⁻¹ | 7.3968 × 10⁻⁵ | μM⁻¹ | 0.7005 | μM |

A composite copper complex (I)-single-walled carbon nanotube coating was prepared for ethylene sensing. At first 0.4 g of NaBH₄ and 7.55 g of [3,5-(CF₃)₂-pyrazol-1-yl](also known as 3,5-(CF₃)₂-pz) were mixed with kerosene in a conical flux to form a homogeneous mixture. The solution was slowly heated to 190° C. with a 1° C./min ramp rate and kept at that temperature for 4 hours. The flux was partially submerged in silicone oil during the heating process. The solution was occasionally (every 15 minutes) heated with a heat gun until pyrazole melted. Next, the solution was cooled down to room temperature and diethyl ether was added to filter out the excess reagents. The resulting white solid was characterized with nuclear magnetic resonance (NMR) imaging to confirm the formation of the product Na[HB(3,5-(CF₃)₂-pz)₃]. Next, 8 mg of copper(I) trifluoromethane sulfonate benzene complex was dissolved in 3 mL dry, degassed toluene. Subsequently, in this solution 17 mg of Na[HB(3,5-(CF₃)₂-pz)₃] was introduced and the solution was stirred for 20 hours at room temperature. The resulting solution was filtrated through a Whatman 0.02 μm syringe filter and a colorless solution of copper complex-1 was obtained.

In a separate tube, 0.5 mg of single-walled carbon nanotube was added to a mixture of 0.8 mL 1,2-dichlorobenzene and 1.16 mL toluene, and the resulting mixture was sonicated for 2 hours to prepare a homogeneous solution. Next, the freshly prepared copper complex-1 solution was added to this mixture and sonicated for another 1 hour. Finally, 30 μL of this solution was drop casted on the working electrode of the ethylene sensor.

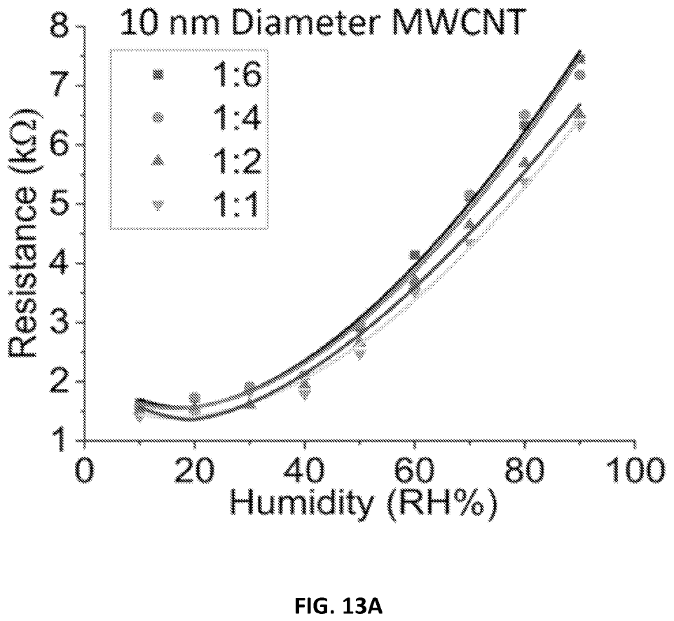

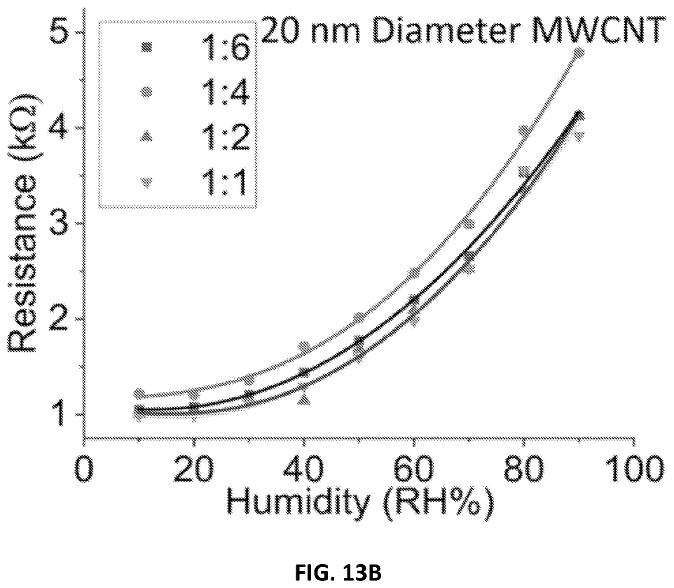

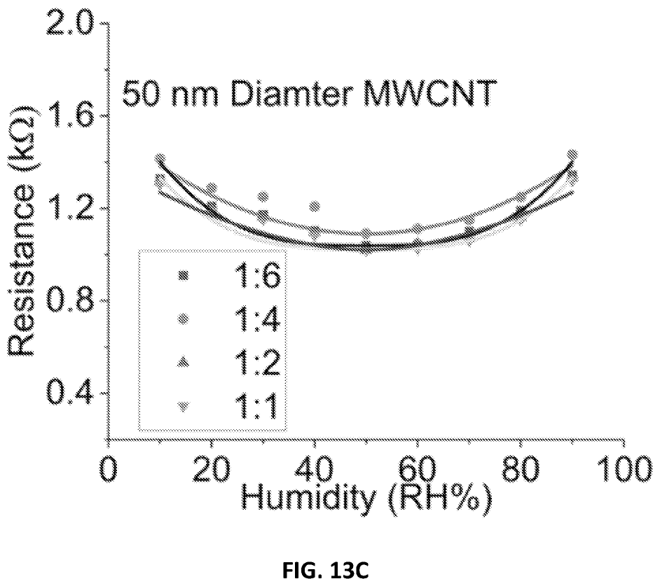

A functionalized multiwalled carbon nanotube-hydroxyethyl cellulose coating was prepared for relative humidity sensing. The selective coating for the humidity sensor was composed of functionalized multiwalled carbon nanotube (f-MWCNT) and hydroxyethyl cellulose (HEC). Modification of MWCNT was done to increase its hydrophilicity and prepare a homogeneous dispersion in an aqueous solution. Therefore, the MWCNT was functionalized with hydroxyl (—OH) groups using an acid treatment. At first 200 mg of MWCNT was added to 3:1(v/v %) mixture of sulfuric acid and nitric acid. Next, this mixture was stirred at 500 rpm for 3 hours while applying 140° C. reflux simultaneously. Afterwards, both the reflux and stirring were stopped, and the mixture was left to cool down to room temperature. Ammonium hydroxide was added until pH of the solution reached 5.5. Next, the f-MWCNT was vacuum filtered using a 0.2 μm PTFE membrane and then heated in the convection oven at 140° C. for 12 hours. Following this step, a 1.2 wt % of f-MWCNT dispersion was made in DI water. To prevent evaporation of DI water, constant magnetic stirring was performed for 3 hours. Consequently, four different weight ratios of f-MWCNT to HEC (1:6, 1:4, 1:2, 1:1) were prepared and stirred for 3 hours. It is noteworthy that HEC is hygroscopic and hence has a high solubility in water, thereby preventing the agglomeration of f-MWCNT. The mixture containing f-MWCNT and HEC was constantly stirred for 30 hours to prepare a homogeneous mixture. Next, 50 wt % of polyvinylpolypyrrolidone (PVPP) was added with a 1:2 binder to filler ratio and the resulting solution was stirred for 5 hours to achieve homogeneity. This ink was evenly applied to the interdigitated electrodes to realize a humidity sensor. The f-MWCNT to HEC ratio of 1:6 (with the length of f-MWCNT being 10 nm) provided the highest sensitivity to relative humidity variations (FIGS. 13A-13C), and Table IV, below.

TABLE IV
Sensitivity and LOD analysis of the different ratio of f-MWCNT:HEC

| | | Low-RH | High-RH | Limit of |
|---|---|---|---|---|
| | Ratio | Sensitivity | Sensitivity | Detection |
| f-MWCNT size | (f-MWCNT:HEC) | kΩ/(% RH) | kΩ/(% RH) | (LOD) |
| 10 nm | 1:6 | 0.011589 | 0.1485 | 11.321% RH |
| | 1:4 | 0.011089 | 0.1324 | 11.541% RH |
| | 1:2 | 0.001056 | 0.1245 | 11.856% RH |
| | 1:1 | 0.0010158 | 0.1215 | 12.057% RH |
| 20 nm | 1:6 | 0.011223 | 0.1365 | 11.258% RH |
| | 1:4 | 0.011258 | 0.1408 | 11.311% RH |
| | 1:2 | 0.010876 | 0.1376 | 11.452% RH |
| | 1:1 | 0.010681 | 0.1357 | 11.523% RH |
| 50 nm (fitted with | 1:6 | 0.0004 | 0.005 | 41.732% RH |
| polynomial) | 1:4 | 0.0003 | 0.0052 | 40.365% RH |
| | 1:2: | 0.0002 | 0.0025 | 45.258% RH |
| | 1:1 | 0.0001 | 0.0011 | 49.568% RH |

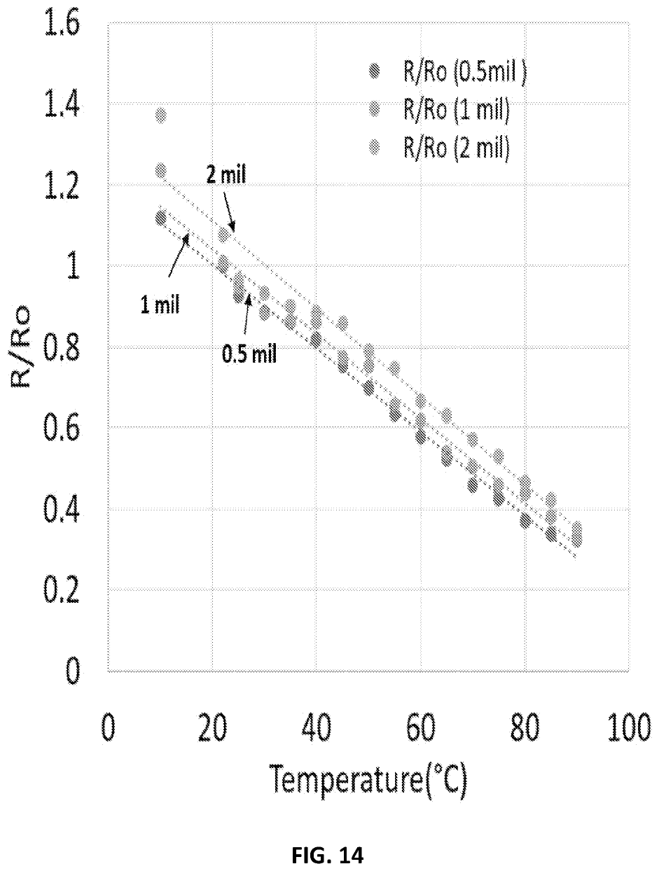

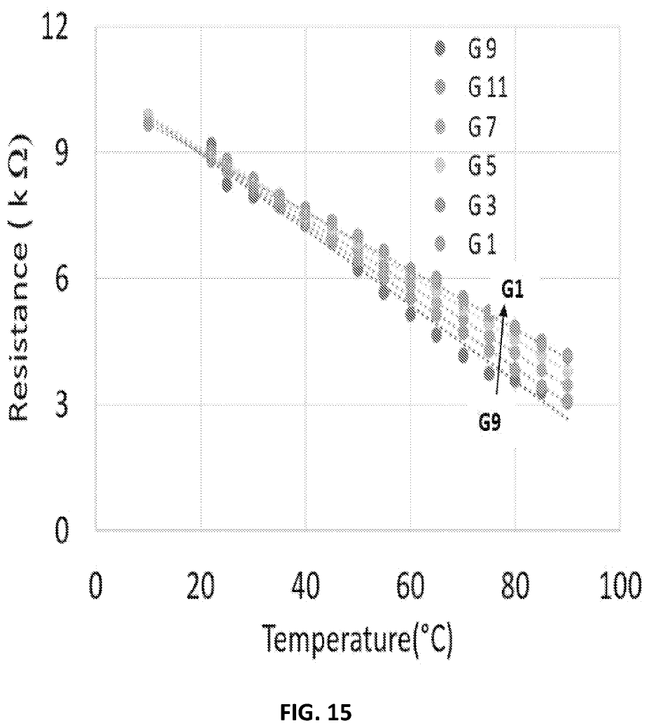

A PEDOT:PSS coating was prepared for temperature sensing. Poly(3,4-ethylenedioxythiophene): poly(styrenesulfonate) (PEDOT:PSS) was selected as the selective coating for temperature sensing. 1.3 wt % of 100 mg PEDOT:PSS was mixed with 50 mg Trion X-100 surfactant and the mixture was centrifuged at 300 rpm for 10 minutes. Different amounts of the 3(glycidyloxypropyl)trimethoxysilane (GOPS) cross-linker was added to this mixture. The weight ratio of PEDOT:PSS to GOPS was varied (1:1, 1:3, 1:5, 1:7, 1:9, and 1:11); calibration curves of the temperature sensor for different weight ratios of PEDOT:PSS to GOPS (1:1(G1), 1:3(G3), 1:5(G5), 1:7(G7), 1:9(G9) and 1:11(G11); FIG. 15). The mixture was centrifuged at 400 rpm for 30 minutes to achieve uniformity and then degassed in a desiccator for 30 minutes to remove the air bubbles from the mixture. Then, 300 μL of this solution was drop casted over the interdigitated electrode of the temperature sensor and subsequently annealed at 140° C. for 60 minutes so that the solvent was entirely evaporated, and the cross-linking was complete. As PEDOT:PSS is sensitive to moisture, Kapton tape of different thicknesses (½ mil, 1 mil and 2 mil) were used to encapsulate the PEDOT:PSS coating. Our experiments showed that the ½ mil Kapton tape resulted in the best performance (FIG. 14). Additionally, it was observed that the PEDOT:PSS to GOPS ratio of 1:9 provided the highest sensitivity to temperature variations (FIG. 15).

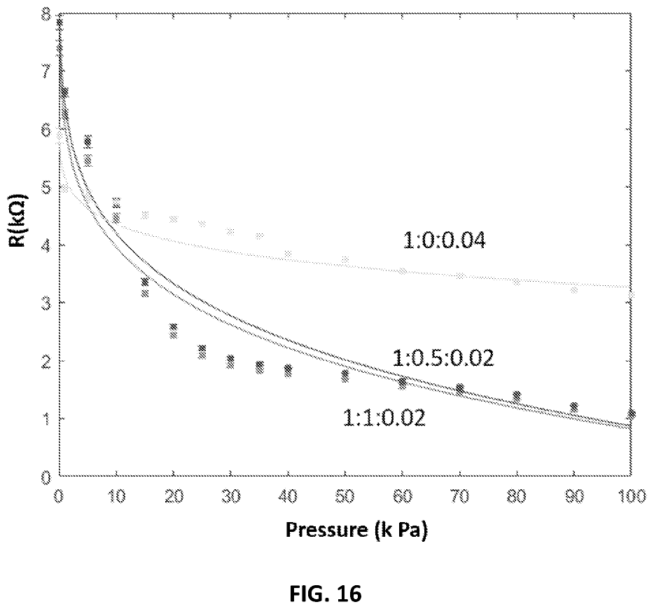

A porous PDMS framework was prepared for pressure sensing. Benzophenone and diphenylamine were mixed at a molar ratio of 1:1 to form a deep eutectic solvent (DES). The eutectic solution had a yellowish color due to charge transfer interactions. Subsequently, a fixed amount of CB was introduced to the yellow deep eutectic solution, which formed a uniform gel owing to the van der walls and π-π interactions between DES and CB. Separately, polydimethylsiloxane (PDMS) polymer was prepared using the standard mixing ratio of 10-parts base elastomer and 1-part curing agent. Then the as-prepared PDMS was added to the DES-CB gel to get a slurry-like ink. Different weight ratios of 1:1:0.02, 1:0.5:0.02, and 1:0:0.04 were experimented to find the optimized ratio of PDMS:DES:CB (FIG. 16). It was found that the 1:1:0.02 ratio of PDMS:DES:CB resulted in the highest sensitivity to pressure changes. This composite ink was drop casted over the interdigitated electrode of the pressure sensor and pre annealed at 75° C. to cure the PDMS followed by a final annealing at 140° C. to remove the DES. The composite ink may have a phase separation between the DES and PDMS due to their immiscibility. During the pre-annealing step, the boiling point of DES was higher than the curing temperature of PDMS. As a result, DES remained as a liquid template in the casted film. When the pre annealed layer was heated to 140° C., DES was evaporated, leaving behind a porous structure in the resulting composite film. Such a porous network offered superior performance in response to applied pressure variations.

A reduced graphene oxide coating was prepared for strain sensing. The strain sensor was made of a reduced graphene oxide (rGO) dispersion. At first rGO was added to N-methyl-2-pyrrolidone (NMP) to make a 2 mg/mL solution. The solution was sonicated for 1 hour. Subsequently, 50 μL of this uniform dispersion was drop casted over the graphene interdigitated electrode. Reduced graphene oxide is sensitive to water vapor. Therefore, the rGO-coated electrode was encapsulated with different thicknesses of Kapton tape (½ mil, 1 mil and 2 mil). The ½ mil Kapton tape demonstrated optimized sensor performance.

### Example 7

Cyclic Voltammetry (CV) and Differential Pulse Voltammetry (DPV) were conducted using the commercially available potentiostat EmStat (PalmSense, Houten, Netherlands). Hitachi™ 4000 plus Scanning Electron Microscope (SEM) and Nicolet Avatar 360 E.S.P ATR-FTIR (Attenuated Total Reflectance-Fourier Transform Infrared) spectrometer were used to characterize the coatings. Applent AT 3817 LCR meter was utilized to measure the resistance (for calibration).

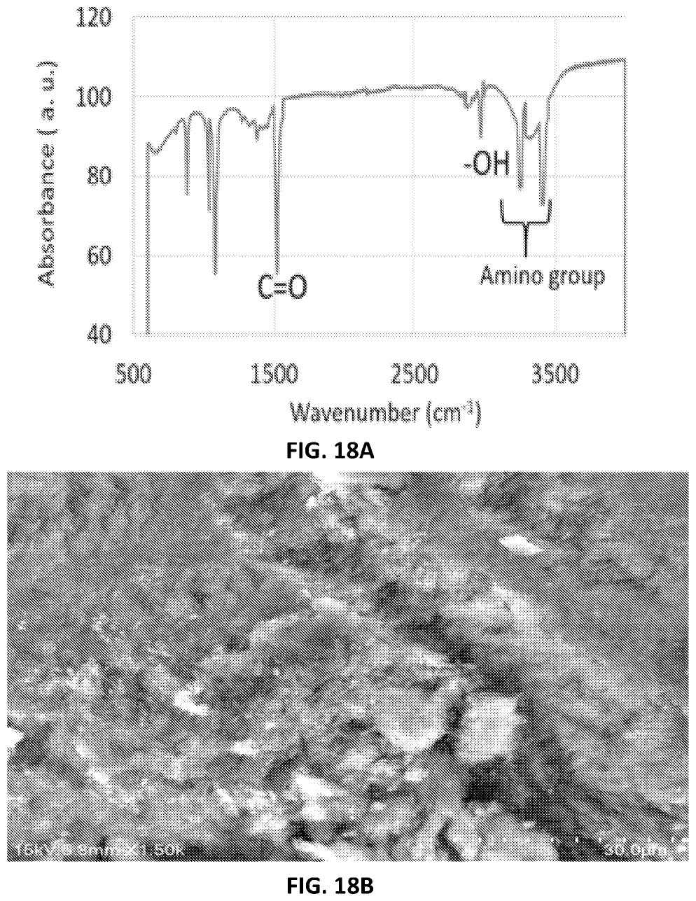

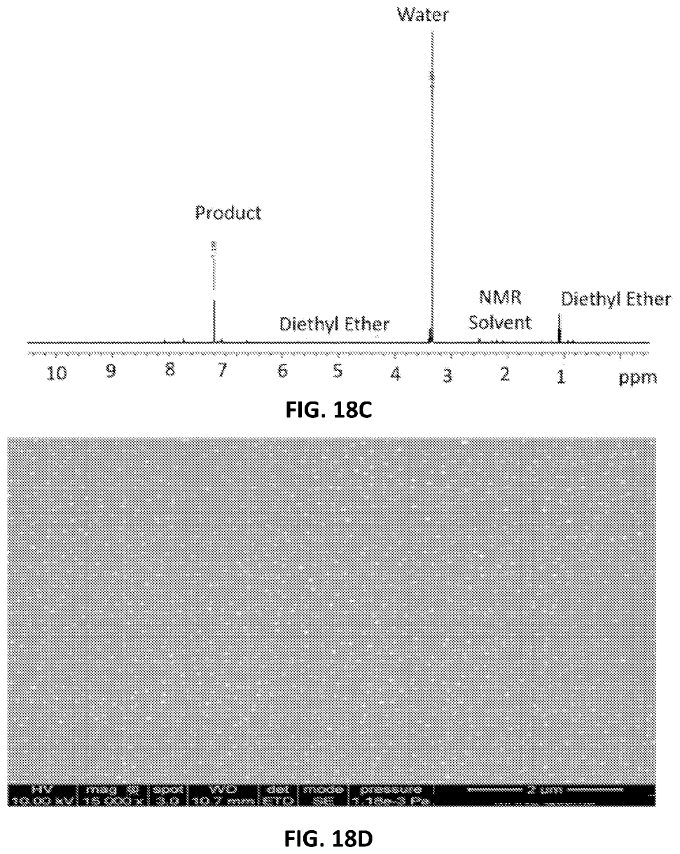

Characterization of the CuMOF coating according to Example 6. The CuMOF coating was characterized with Fourier Transform Infrared (FTIR) spectroscopy, as shown in FIG. 18A. The peaks at 3550 cm⁻¹ and 3390 cm⁻¹ were due to the asymmetric and symmetric stretching vibrations of —NH₂, confirming the presence of amino groups in CuMOF. The stretching vibration peak that appeared at 2950 cm⁻¹ for —OH was mitigated by the interactions between Cu²⁺ ion and —COOH of 2-aminoterephthalic acid. The FTIR spectra of the CuMOF coating agreed with the previous reports. The scanning electron microscopy (SEM) image of the composite CuMOF-CB-Nafion coating is shown in FIG. 18B.

One crucial step in the synthesis of the copper complex (I) coating was accurate formation of the intermediate product Na[HB(3,5-(CF₃)₂-pz)₃]. Hence, formation of this intermediate product was verified through the NMR spectroscopy, as depicted in FIG. 18C. The spectroscopic results confirmed the presence of product Na[HB(3,5-(CF₃)₂-pz)₃]. Furthermore, the SEM image demonstrated size, distribution, and morphology of the copper complex (I) nanoparticles deposited on the graphene-coated electrode surface (FIG. 18D). The nanoparticles had a mean diameter of about 10 nm.

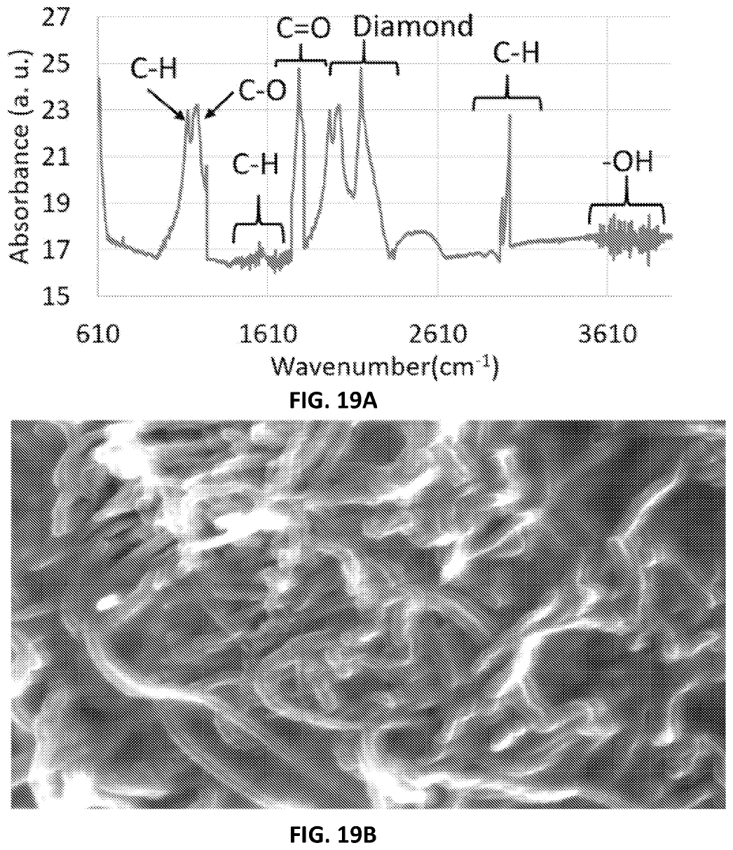

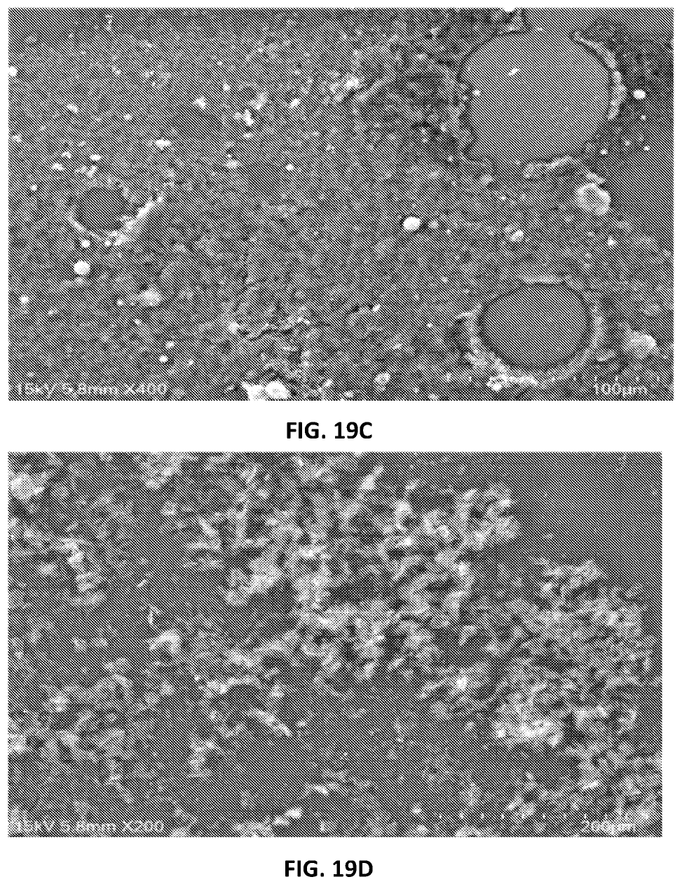

The f-MWCNT was characterized with FTIR (FIG. 19A). The absorption peaks between 3500 cm⁻¹ and 4000 cm⁻¹ were due to the presence of —OH functional groups. The peaks located between 2975-3065 cm⁻¹, 1390-1700 cm⁻¹ and 1100-1160 cm⁻¹ confirmed the presence of C—H bonds. The unexpected peaks that occurred between 1950 to 2210 cm⁻¹ represented the artifacts of the diamond ATR setup. The FTIR spectra of the f-MWCNT coincides with the previous reports. The SEM image in FIG. 19B shows the morphology of the HEC/MWCNT coating.

FIG. 19C shows the SEM image of the porous PDMS network formed by first trapping and then evaporating the deep eutectic solvent. The pores had diameters ranging from 10 μm to 400 μM (FIG. 19C). FIG. 19D shows an SEM image of the reduced graphene oxide (rGO) bundles.

### Example 8

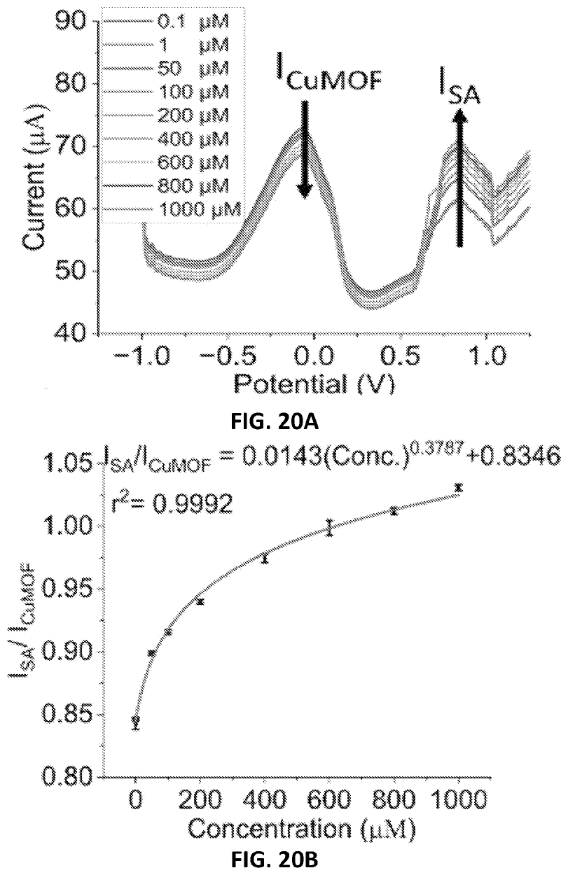

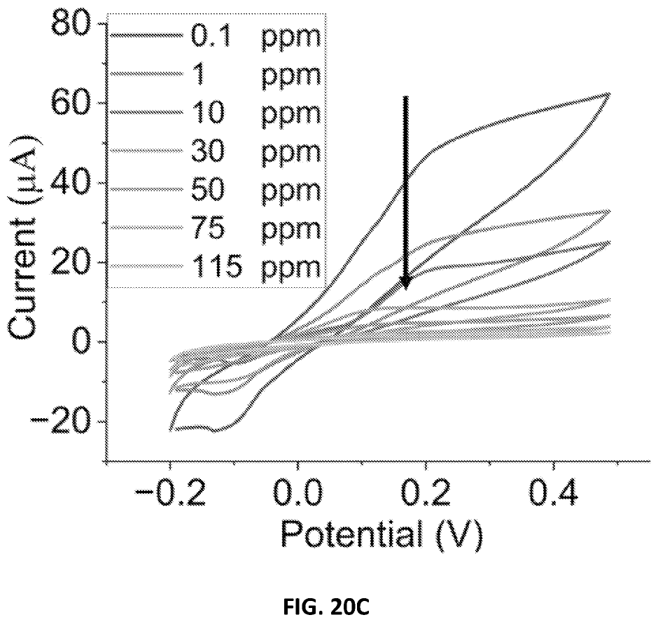

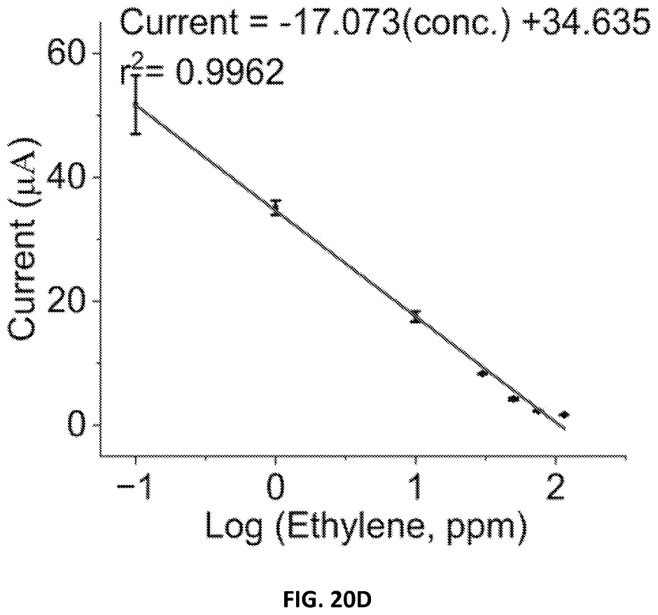

Calibration of the sensors fabricated and coated in Examples 5-6. Electrochemical characterizations were performed for both salicylic acid and ethylene sensors. First, differential pulse voltammetry (DPV) was performed in a 0.05 M tris HCl buffer (pH=7.1) to characterize the salicylic acid sensor. The DPV test was performed in the voltage range from −1.0V to 1.5V with a 0.01V step and scan rate of 10 mV/s. The magnitude and duration of the pulse (Eₚᵤₗₛₑ and tₚᵤₗₛₑ) were 0.3V and 0.1 is, respectively. The SA sensor was calibrated with 0.1 μM, 1 μM, 50 μM, 100 μM, 200 M, 400 μM, 600 μM, 800 μM and 1000 M of SA. The DPV response had two redox peaks, as shown in FIG. 20A. The current peak (I_{CuMOF}) located at approximately −0.2V was due to the reduction of Cu²⁺ in the CuMOF coating, whereas the peak (I_{SA}) at 0.85V was attributed to salicylic acid oxidation by the composite coating. It is noteworthy to mention that with increasing concentrations of SA, the redox peak current at 0.85V increased. Simultaneously, the Cu²⁺ peak current diminished owing to the increased reduction of Cu²⁺ ion. Because of the considerable separation of 1.05V between the Cu²⁺ and the SA peaks, the ratio of two peak currents was used as the sensor response. FIGS. 20A and 20B show the DPV responses and the corresponding calibration curve fitted with a power series.

Cyclic voltammetry (CV) was used to characterize the ethylene sensor. The CV was performed from −0.2V to 0.5V at the scan rate of 50 mV/s, with Eₛₜₑₚ=0.01V. The sensor was exposed to varying concentrations of gaseous ethylene, such as 0.1 ppm, 1 ppm, 10 ppm, 30 ppm, 50 ppm, 75 ppm, and 115 ppm. The CV plots shown in FIG. 20C indicate that the redox peak occurred between 0.13V to 0.20V. Upon exposure to ethylene, copper complex (I) bound with ethylene to form a second complex, which had a limited interaction with the single-walled carbon nanotube. As a result, the conductivity of single-walled carbon nanotube decreased and hence current decreased. With an increased concentration of ethylene, there was a proportional reduction in the peak current (FIG. 20C). The peak current values were plotted as a function of ethylene concentrations to generate the calibration curve shown in FIG. 20D. The peak current showed a linear response to varying ethylene concentrations.

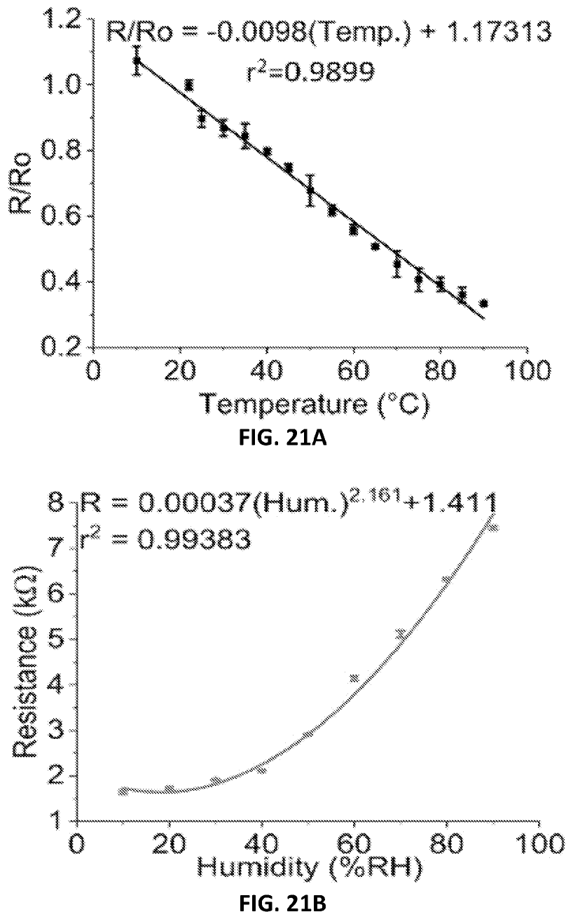

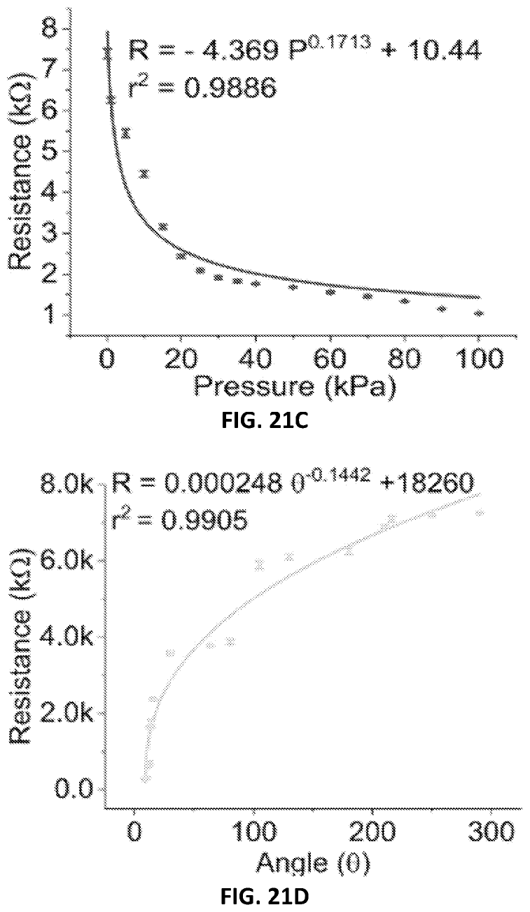

The temperature, humidity, pressure and strain sensors were resistive by nature. This attributed to resistance variations in response to temperature, humidity, pressure, or strain variations. The temperature sensor was fabricated with a Poly(3,4-ethylenedioxythiophene): poly(styrenesulfonate) (PEDOT:PSS) coating, the relative humidity sensor was composed of functionalized multiwalled carbon nanotube-hydroxyethyl cellulose coating, the pressure sensor had a porous polydimethylsiloxane-deep eutectic solvent-carbon black (PDMS:DES:CB) framework, and the strain sensor was made with reduced graphene oxide (rGO). The sensors were calibrated with an LCR meter, capable of measuring inductance, capacitance, and resistance at an operating frequency of 100 Hz. The temperature sensor was calibrated with temperature values ranging from 10° C. to 90° C. As PEDOT:PSS has a negative temperature coefficient of resistance, its resistance decreased with increasing temperature. The calibration curve of the temperature sensor showed a high degree of linearity with a Pearson coefficient of 0.9899 (FIG. 21A). Next, the humidity sensor was calibrated for relative humidity values ranging from 10% to 90%. With increasing relative humidity, the resistance also increased, as demonstrated in FIG. 21B. The resistance versus relative humidity measurements were fitted with a power series having r²=0.9789.

Similarly, the pressure sensor was calibrated with various pressure values ranging from 0.1 kPa to 100 kPa. As the pressure increased, the resistance of the pressure sensor decreased, confirming the negative pressure coefficient of resistance of PDMS:DES:CB (FIG. 21C). The strain sensor was calibrated for various angles of curvature. The equation that relates the angle of curvature and radius is given by Formula (4). Where s, r, and θ represent arc length, radius, and angle of curvature, respectively. Here, the arc length, s, is the same as the length of the sensor (2 cm). Various radii (r=1.8 cm, 1.43 cm, 1.25 cm, 1.09 cm, 0.88 cm, 0.72 cm, 0.63 cm, 0.54 cm, 0.53 cm, 0.45 cm, and 0.4 cm) values were measured to cover the stems of small plants such as bell pepper (stem diameter=0.6 cm), cucumber (stem diameter=−1.1 cm), squash (stem diameter=1.3 cm), tomato (stem diameter=1.34 cm), and maize (stem diameter=2.8 cm). As the stem grows radially, the strain sensor encounters a proportional change in its resistance. The resistive response of the sensor was tested for different angles of curvature ranging from 8.98° to 290°. As the angle of curvature decreased, the radius increased according to Formula (4) and the resistance of the strain sensor decreased. The r values were substituted into Equation (1) to get the angles of curvature ranging from 8.98° to 290° The calibration curve for the strain sensor is shown in FIG. 21D.

The gauge factor

$$( GF = \frac{\frac{Δ R}{R}}{ε} ) ,$$

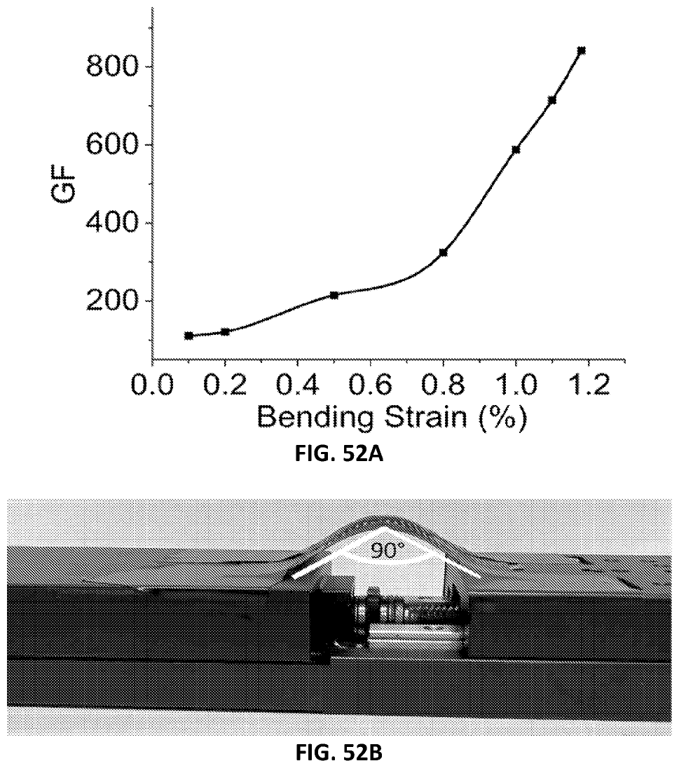

defined as the ratio of relative change in sensor resistance to the mechanical strain, was found to be 842 under a bending strain of 1.4%. Equation 2 shows the equation for the mechanical bending strain,

$$ε = \frac{t}{2 {r}_{b}} ,$$

where t is the combined thickness of the polyimide sheet and the overlaid sensing layers (=127 μm) and r_{b} is the bending radius of the sensor under the bending state. The bending radius was calculated as described above. FIG. 52A shows the gauge factor versus the bending strain plot. A motorized translation stage (MTS50-Z8, Thorlabs Inc., Newton, NJ, USA) was used to measure the bending radius, as illustrated in FIG. 52B. The resolution, defined as the smallest detectable change in the angle of curvature in response to the radial growth in the stem, was calculated to be approximately 0.02°.

Sensitivity and LOD Analysis. The calibration curves for salicylic acid, humidity, pressure, and strain sensors were fitted with power series (y=ax^{b}+c). The sensitivity, S_{y}|ₓ, was calculated using

$${{S}_{y} \vert}_{x} := \frac{dy}{dx} = {abx}^{b - 1}$$

where x and y represent the target parameter (i.e., SA concentration/RH/pressure/angle of curvature depending on the sensor type) and sensor response, respectively, while a and b denote parameters of the fitted curve. Sensitivity values were calculated at both the lowest and highest x values. In contrast, ethylene and temperature sensors exhibited a linear response (FIG. 20D and FIG. 21A) and hence the slope (m) of the linear fit (y=mx+c) was used as a measure of sensitivity.

The limit of detection (LOD) for the physical sensors, i.e., temperature, humidity, pressure, and strain sensors, was calculated using the following formula

$$LOD = \frac{3 * std . dev .}{Sensitivity} .$$

The LOD for chemical sensors, i.e., SA and ethylene sensors, was calculated using the following sets of equations:

$$LOB = mean of signal ( blank sample ) + 1.645 ( std . dev . of blank sample ) \qquad ( 5 )$$

$$yLOD = LOB + 1.645 ( std . dev . of target at low concentration ) \qquad ( 6 )$$

$$LOD = \frac{yLOD - intercept}{slope} \qquad ( 7 )$$

Tables V and VI summarize the sensitivity, LOD, and resolution for all the sensors.

TABLE V
Performance metrics of non-linear sensors.

| | | Low | High | | | | |
|---|---|---|---|---|---|---|---|
| | | Concentration | Concentration | | | | |
| Sensor | Equation | Sensitivity | Sensitivity | LOD | | Resolution | |
| Salicylic | I_{SA}/I_{CuMOF} = | 0.002264 | 7.409 × 10⁻⁵ | 0.644 | μM | 0.301 | μM |
| acid, SA | 0.0143(SA)^{0.3787} + | μM⁻¹ (at 0.1 | μM⁻¹ (at 1000 | | | | |
| | 0.8346 | μM) | μM) | | | | |
| Humidity, | R = 0.0037 | 0.011589 kΩ/(% RH) | 0.1485 kΩ/(% RH) | 11.321% | RH | 0.245% | RH |
| RH | (RH)^{2.161} + 1.411 | (at 10% RH) | (at 90% RH) | | | | |
| Pressure, | R = −1.369 P^{0.1713} + | 5.04 kΩ/ | 0.0164 kΩ/ | 0.3733 | kPa | 0.11 | kPa |

| | | | | | |
|---|---|---|---|---|---|
| P | 10.44 | (kPa) (at 0.1 kPa) | (kPa) (at 100 kPa) | | |
| Strain, θ | R = 0.000248 | 1.5935 × 10⁻⁶ | 1.006 × 10⁻⁸ | 9.3211° | 0.02° |
| | θ^{0.1442} + 18260 | kΩ/° (Gauge | kΩ/° (Gauge | (0.1%) | |
| | | factor = 100) | factor = 900) | | |

TABLE VI
Performance metrics of linear sensors.

| Sensor | Equation | Sensitivity | LOD | Resolution |
|---|---|---|---|---|
| Ethylene, | I = − 17.073 | 17.073 | 0.6089 | 0.424 ppm |
| ET | (ET) + 34.635 | μA/log(ppm) | ppm | |
| Tempera- ture, T | $\frac{R}{R o} = 0.0098 ( T )$ | 0.0098/° C. | 10.5478° C. | 1.03°C |

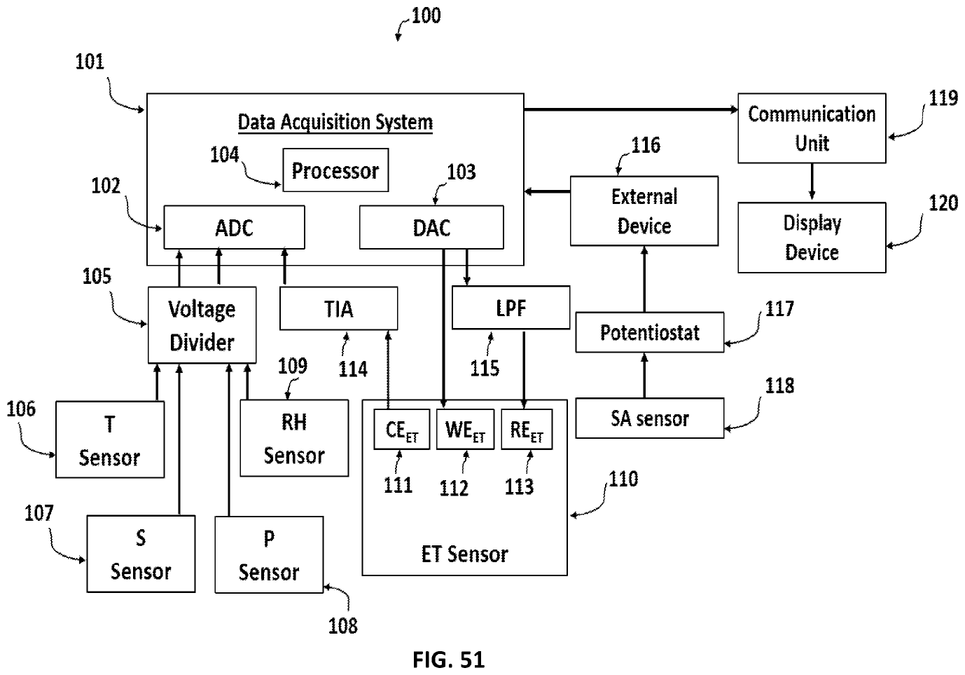

A system 100 was used as shown in FIG. 51, including a voltage divider circuit 105 to measure the resistance variations of the temperature (T) using a temperature sensor 106, relative humidity (RH), using an RH sensor 109, pressure (P), using a pressure sensor 108, and strain sensor (S), using a strain sensor 107, and a potentiostat circuit capable of conducting cyclic voltammetry on the ethylene (ET) sensor 110, including a counter electrode 111, a working electrode 112, and a reference electrode 113. An ESP32 microcontroller with in-built communication unit 119 (i.e., a WiFi capable unit) served as the main processing unit, shown as a processor 104 and memory (not shown), of the data acquisition system (DAS) 101. The resistance variations of the T, RH, P, and strain sensors (106, 109, 108, and 107) were converted to voltage measurements via a voltage divider circuit 105. A constant voltage of 3.3V was applied across the voltage divider 105 and the auto-ranging functionality was adopted to select a known resistor from a specifically identified range. The DAS had an in-built analog-to-digital converter (ADC, 102), which read the voltage across the T, RH, P, and strain sensors (106, 109, 108, and 107) and converted the analog voltages to digital values.

To obtain cyclic voltammetry measurements from the ET sensor 110, an 8-bit digital-to-analog converter (DAC, 103) generated a staircase voltage waveform (the excitation signal for cyclic voltammetry) and applied that across the working (WE_{ET}, 112) and reference (RE_{ET}, 113) electrodes of the ET sensor. Two staircase waveforms were generated by two DAC modules to verify the accuracy of the excitation signal. Primarily, the timing sequence of the two staircase waveforms was measured. The timing sequence that had less than 1% deviation compared to the software timer, was selected as the excitation signal to run cyclic voltammetry. The DAS 101 had an in-built DACs 103 to perform this operation. A low pass filter (LPF, 115) was added between the DAC 103 and the reference (RE_{ET}, 113) electrode of the ET sensor 110 to remove high-frequency noise from the signal. A trans-impedance amplifier (TIA, 114) converted the analog current measured across the working (WE_{ET}, 112) and counter (CE_{ET}, 111) electrodes of the ET sensor 110 into a voltage signal that was read by the in-built ADC 102 of the DAS 101.

For the T, RH, P, strain (106, 109, 108, and 107), and ET sensor 110, direct memory access (DMA) operation was used to transfer the intermediate ADC reading to the specified memory space. The voltage value, V, was calculated from the ADC reading following the equation

$$V = \frac{ADC Reading * DC input voltage}{{2}^{n} - 1}$$

(where, DC input voltage=3.3V and n=8). Data from all the sensors were processed by the processor 104 to estimate the unknown T, RH, P, strain, and ET measurements from previously stored calibration plots.

The SA sensor 118 employed a ratiometric approach wherein the ratio of the two oxidation peak currents was used as the response signal. Due to this detection method, differential pulse voltammetry (DPV) was found more suitable to investigate the electrocatalytic activity of the SA sensor. A potentiostat 117 (i.e., a commercially available EmStat), connected to an external device 116 (e.g., a laptop computer) was used to conduct DPV measurements. In some embodiments, a DPV circuit may instead be integrated into the DAS.

The hormone levels (SA and ET), temperature, humidity, pressure, and stem diameter measurements were sent to the cloud wirelessly via the communication unit 119 and accessed on a display device 120 (e.g., a smartphone application via the Blynk IoT interfacing).

In an example using the above described system, two plants were measured, wherein plant 1 was unstressed and plant 2 was subjected to water deficiency from Day 1. The water-stressed plant showed higher levels of SA and ET on Day 5 as compared to the measurements observed on Day 1. However, the change in VPD levels was nearly the same in both plants, indicating the potential of the hormone levels in providing an early indication of water deficiency. In some embodiments, the system is configured to display one or more prompts in response to the measured hormone levels, e.g., a “Needs Water” prompt on the display device.

### Example 9

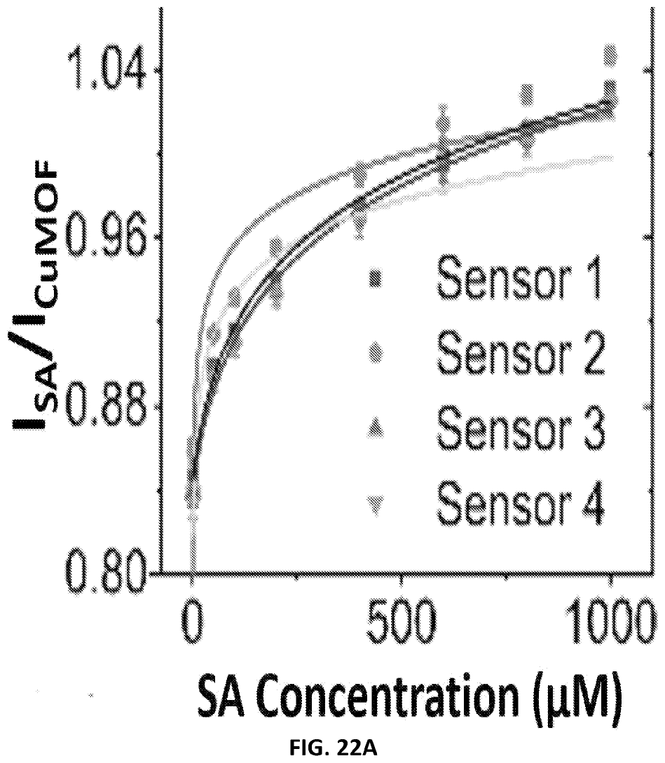

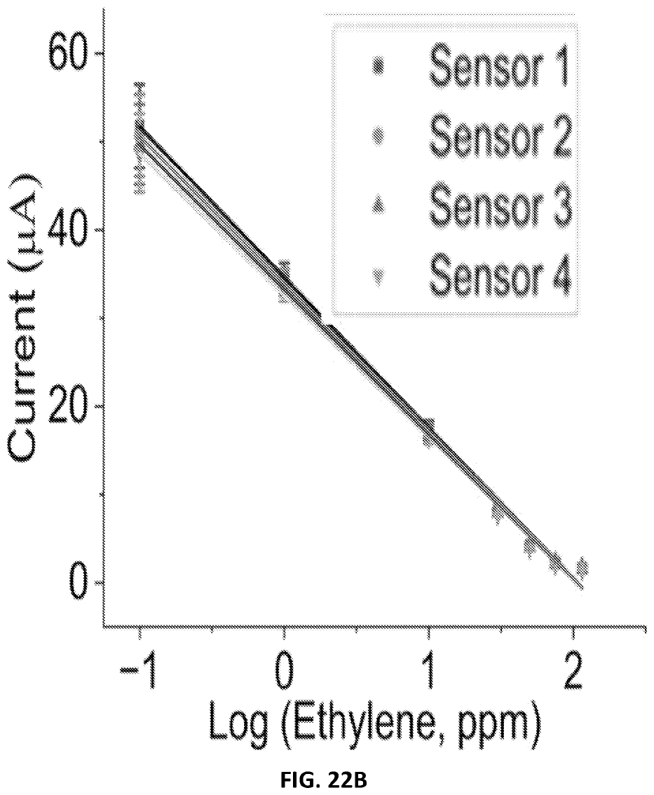

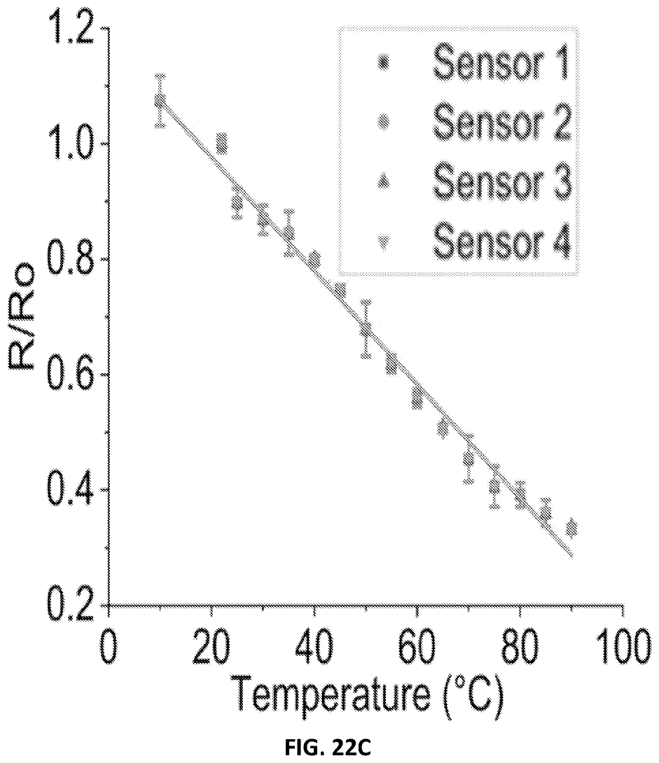

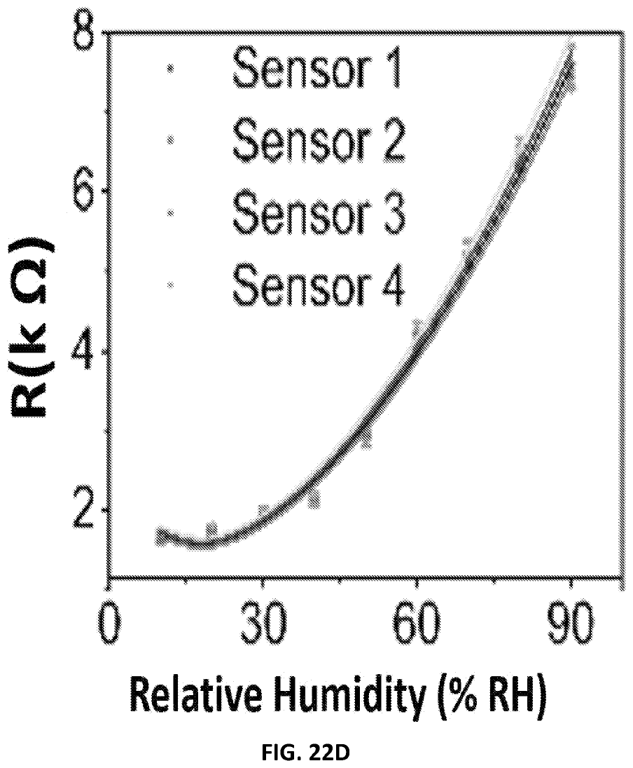

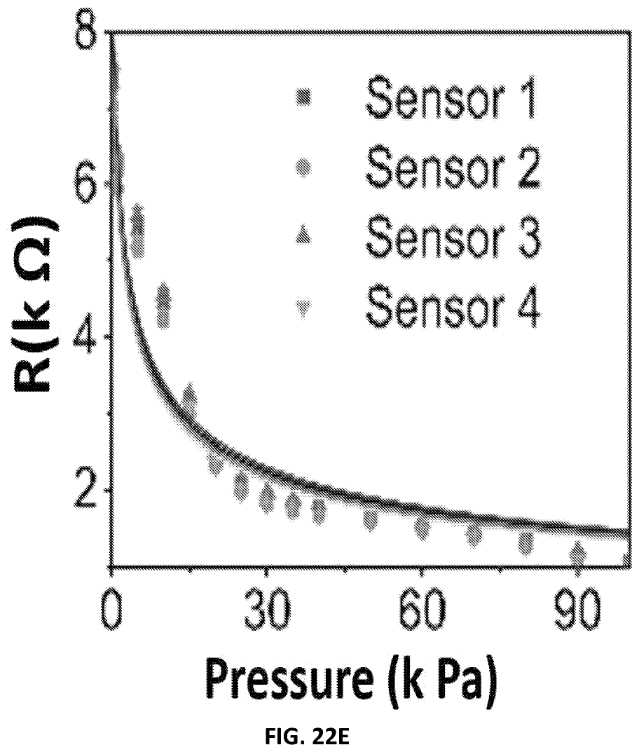

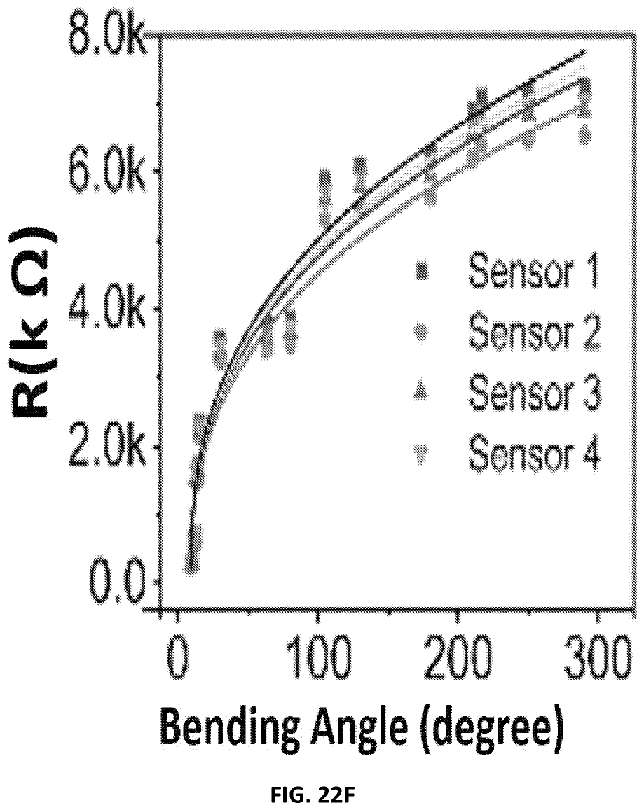

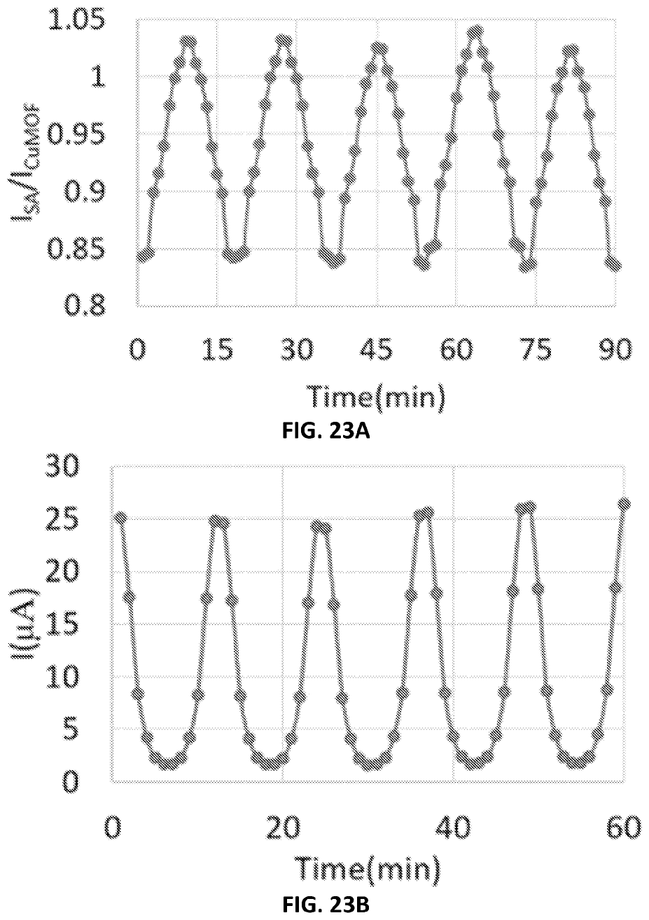

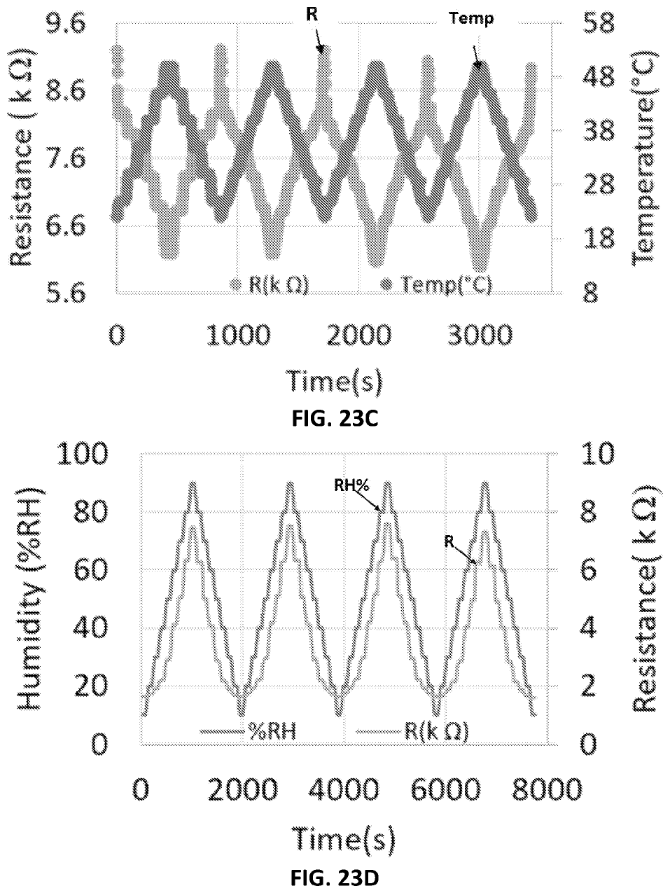

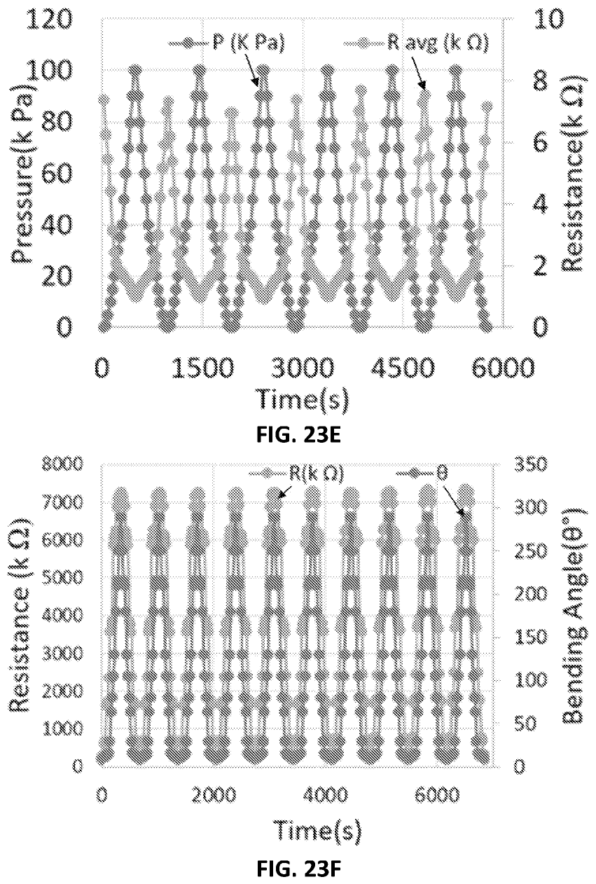

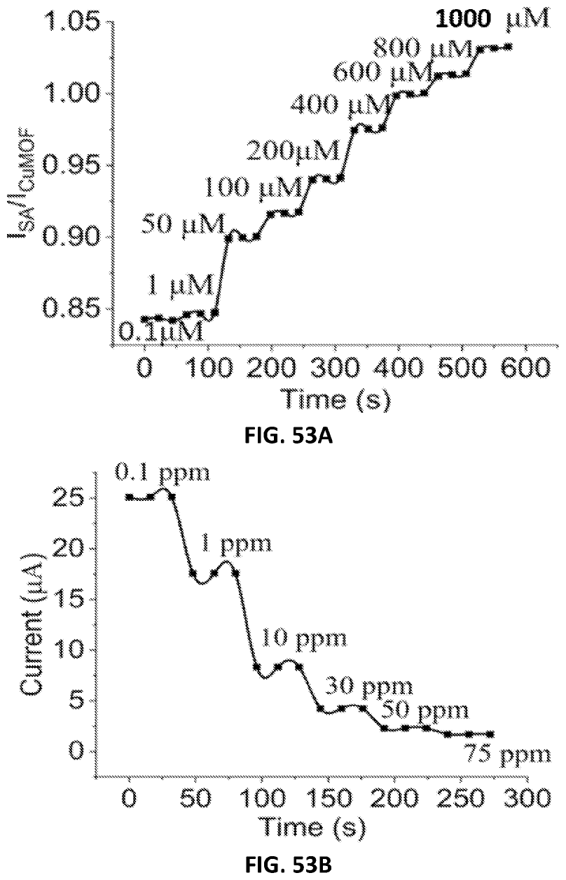

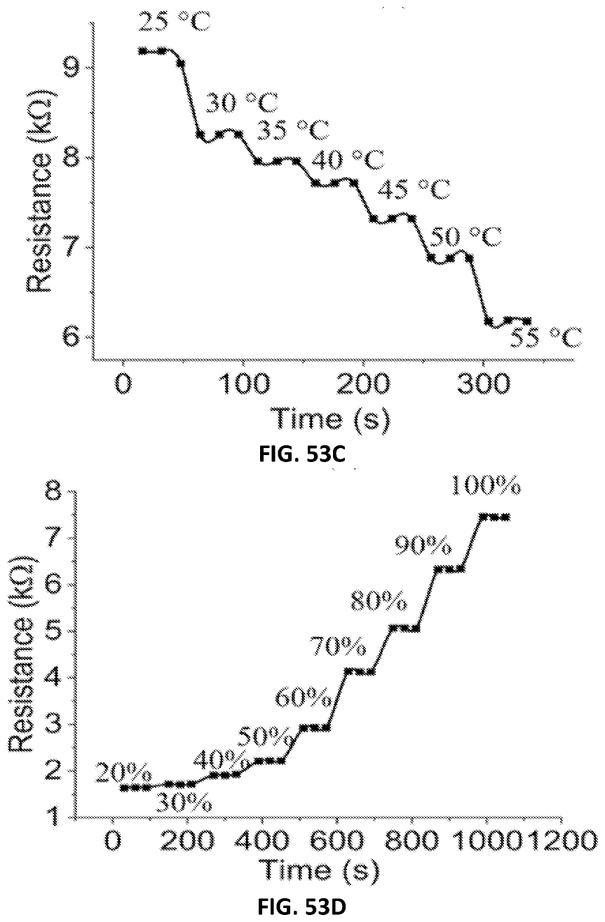

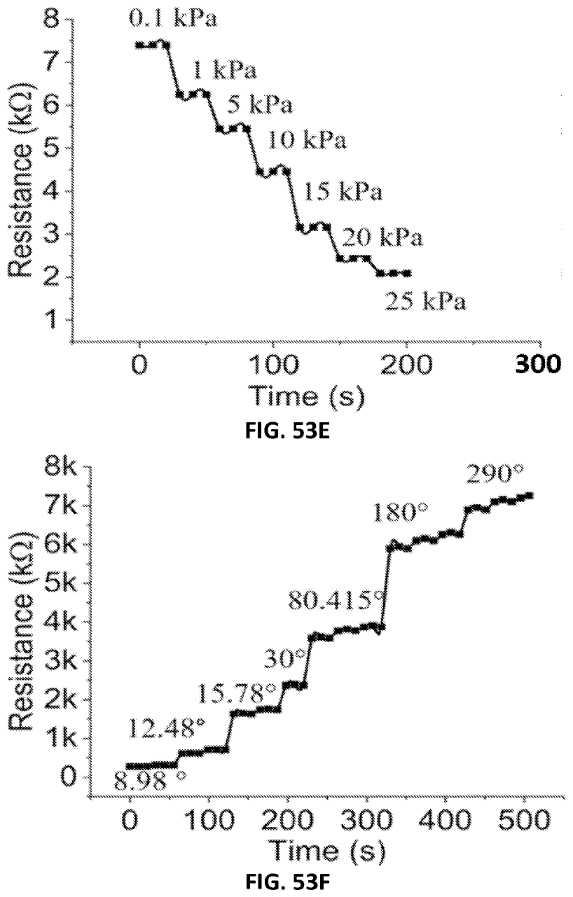

Repeatability, Reproducibility, Bending, and Hysteresis Tests. All the six sensors were characterized for repeatability, reproducibility, bending, and hysteresis, to confirm their feasibility of field deployment. A system as described in FIG. 51, with six sensors, was tested for repeatability, reproducibility, bending, and hysteresis, to confirm their feasibility of field deployment. Reproducibility was tested by repeating the calibration with sensors from each category FIGS. 22A-22F. The coefficient of variance for four repeated measurements was found to be less than 3%. The SA concentration values used for the repeatability test were 0.1 μM, 1 μM, 50 μM, 100 μM, 200 μM, 400 μM, 600 μM, 800 μM, and 1000 μM. A similar procedure was adopted for investigating the repeatable characteristics of other sensors. All the sensors demonstrated a coefficient of variance of less than 5%, which is reasonable for in-field operation. FIGS. 53A-53F show the dynamic response of all sensors over half cycle. The responses were measured using a system as depicted in FIG. 51 with sensors attached to a leaf and stem of a plant, respectively. All the sensors demonstrated a rapid response time of less than a minute. Reproducibility was tested by repeating the calibration for sensors from each category (FIGS. 22A-22F). The coefficient of variance for four repeated measurements was found to be less than 3%. The sensors also demonstrated repeatable characteristics under cyclic variations in salicylic acid (FIG. 23A), ethylene (FIG. 23B), temperature (FIG. 23C), humidity (FIG. 23D), pressure (FIG. 23E), and strain (FIG. 23F) measurements, with coefficient of variance less than 5%, which is reasonable for in-field operation.

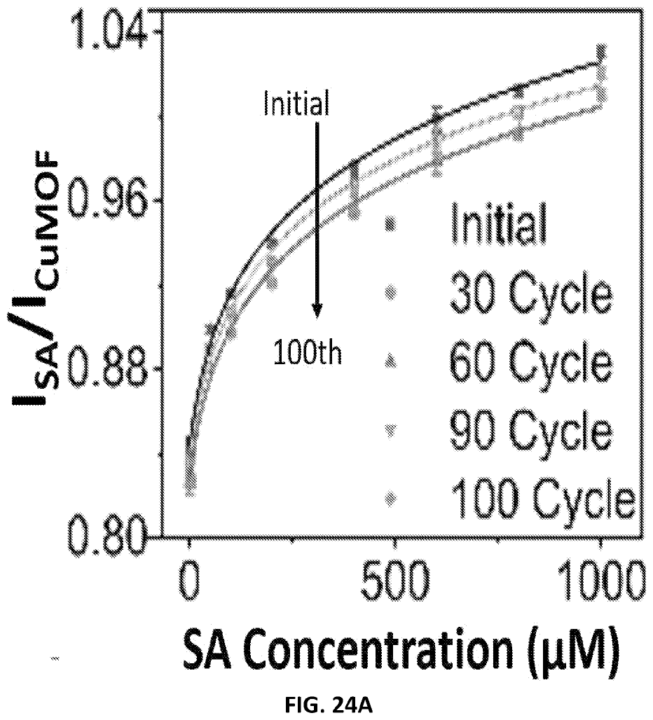

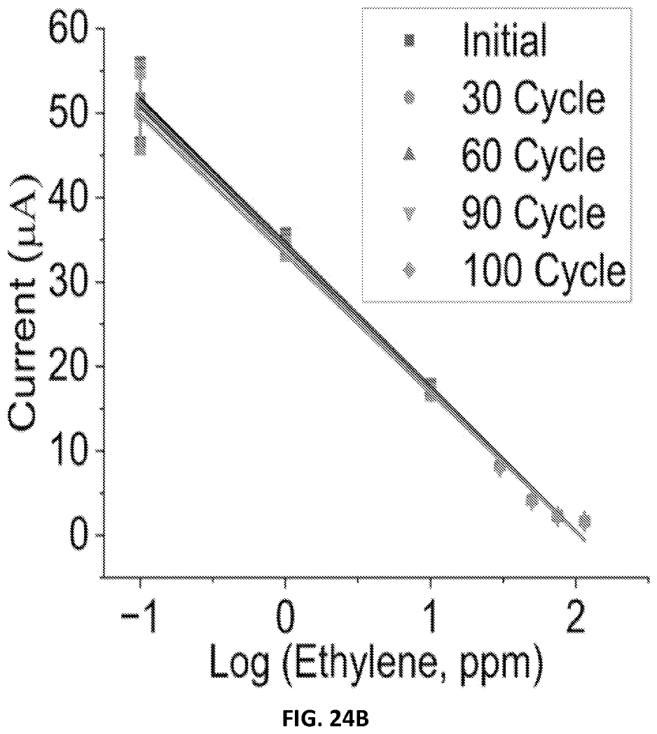

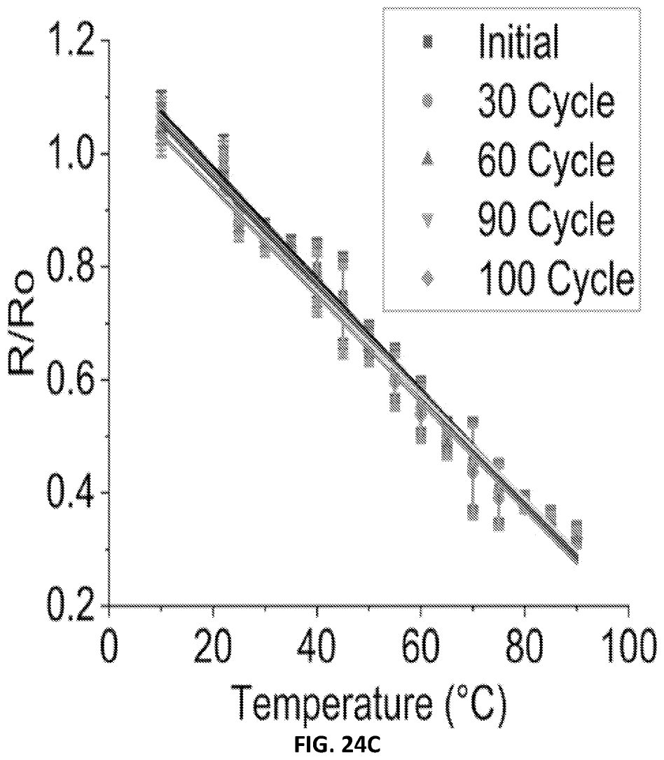

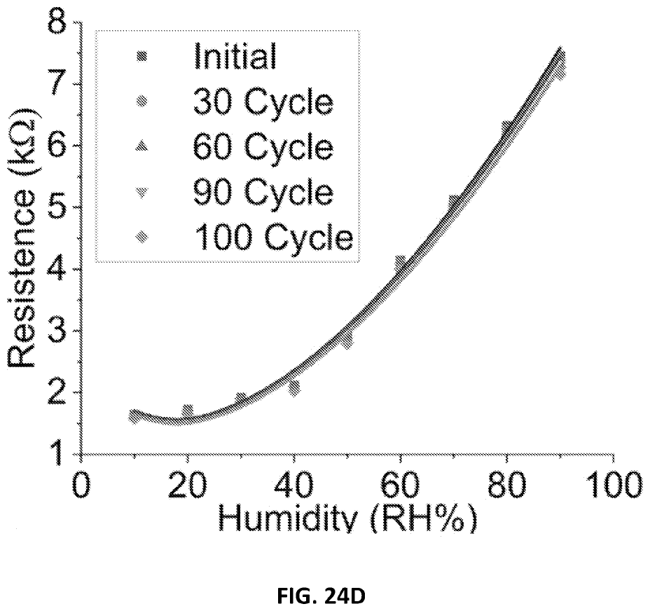

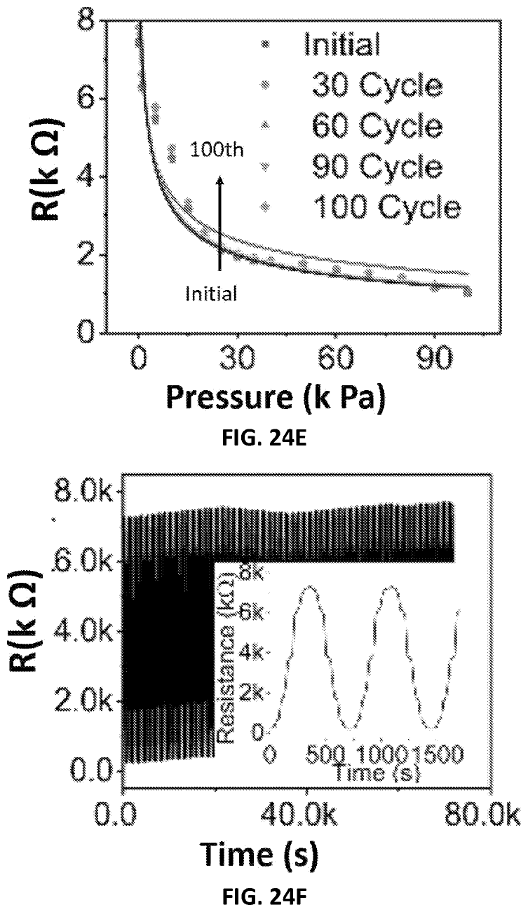

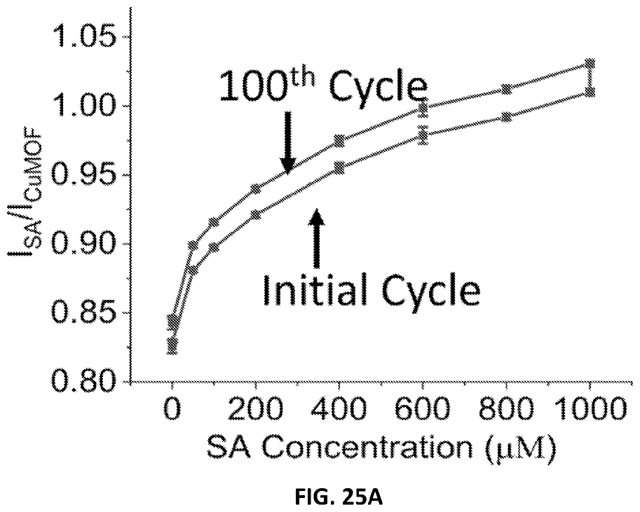

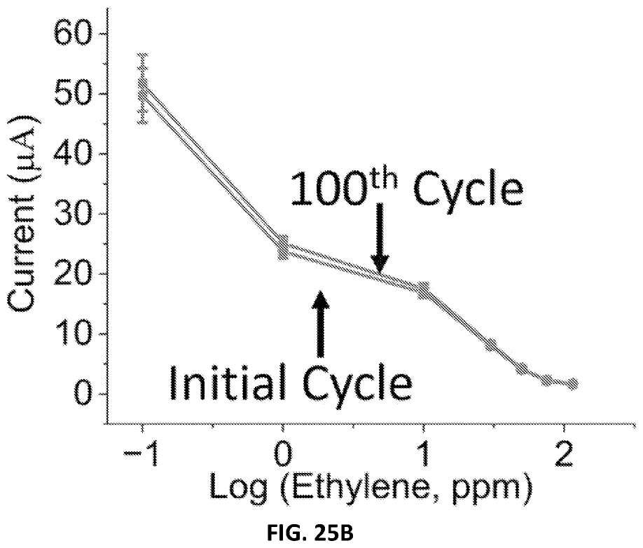

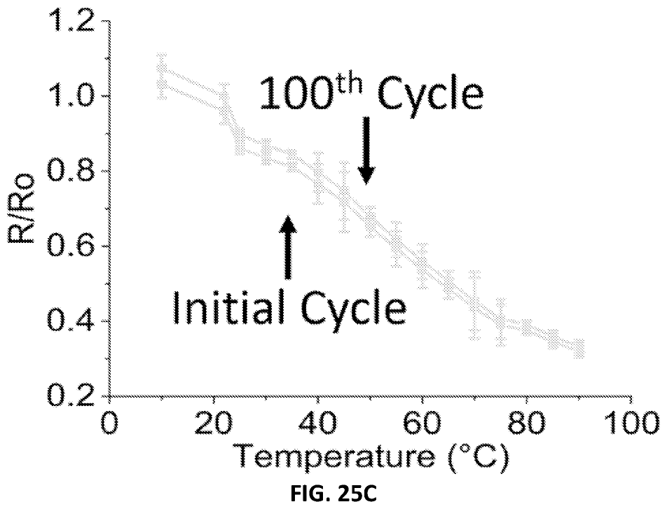

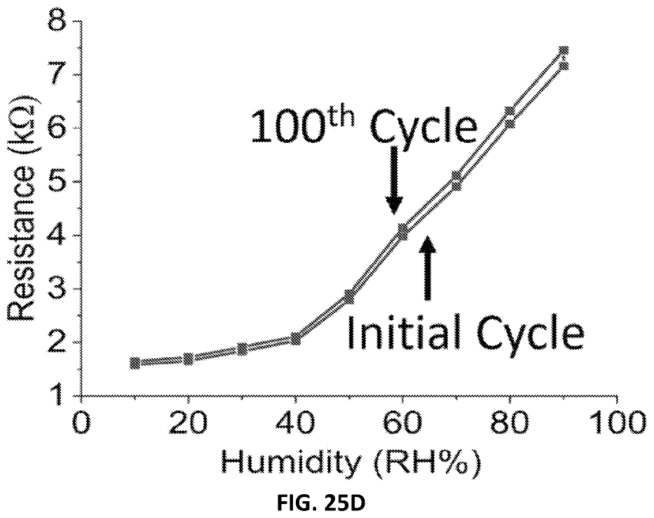

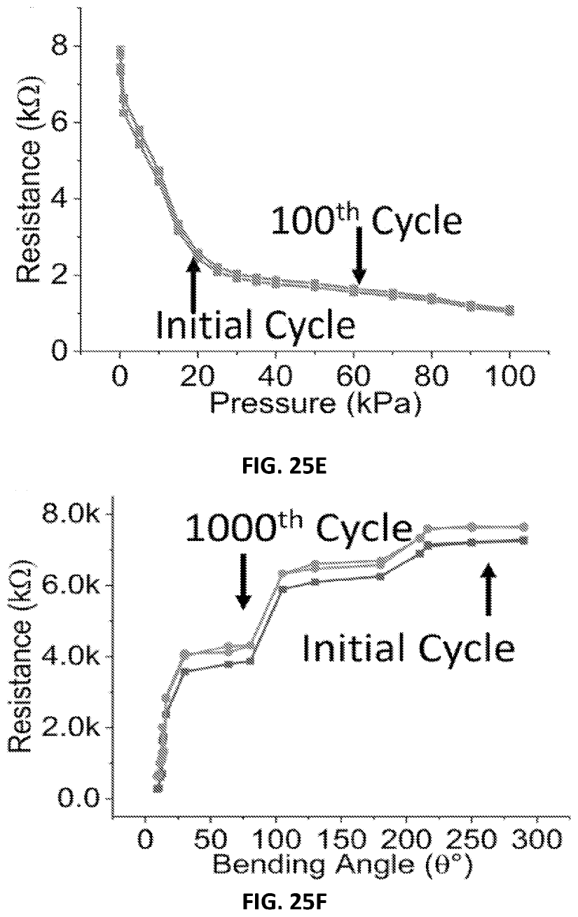

Since all the sensors were made on a flexible substrate, the next test was to bend the substrate at 450 angle up to 30, 60, 90, and 100 cycles and repeat the calibration (FIG. 24A-24F). It was observed that even after bending up to 100 cycles, the coefficient of variance between the calibration curves were less than 3%. The hysteresis between 0^{th} and 100^{th} cycles of bending was calculated to be less than 3% for all but the ethylene sensor (FIG. 25A-25F). A higher hysteresis for the ethylene sensor could be attributed to the deformation of the Nafion sheet after 100 cycles of bending, which resulted in degradation in the sensor performance. Generally, 45° bending of the leaf surface is not expected in a live plant. Yet, a separate correction factor was introduced to account for such performance degradation under large bending angles, as is described in the next section. The response of the strain sensor was recorded for 1000 cycles of repeated bending, as shown in FIG. 25F. Nevertheless, the hysterics was found to be less than 3% even after subjecting the strain sensor to 1000 cycles of bending.

### Example 10

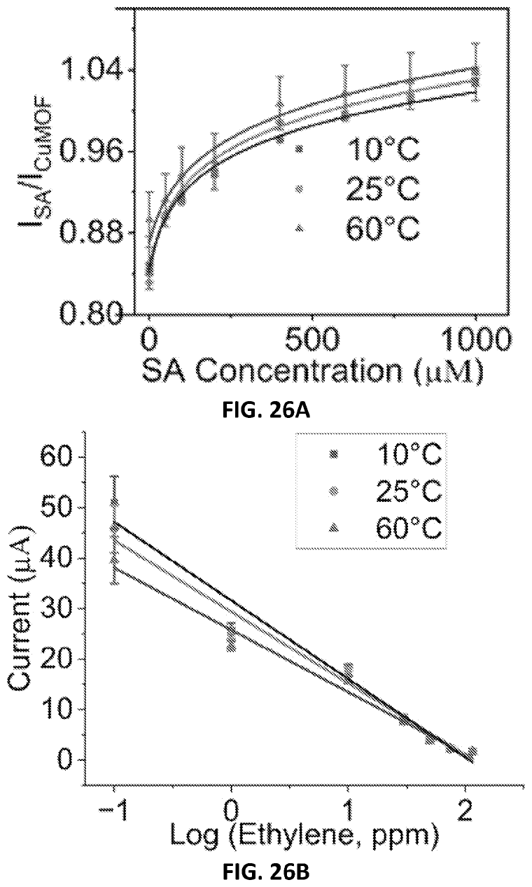

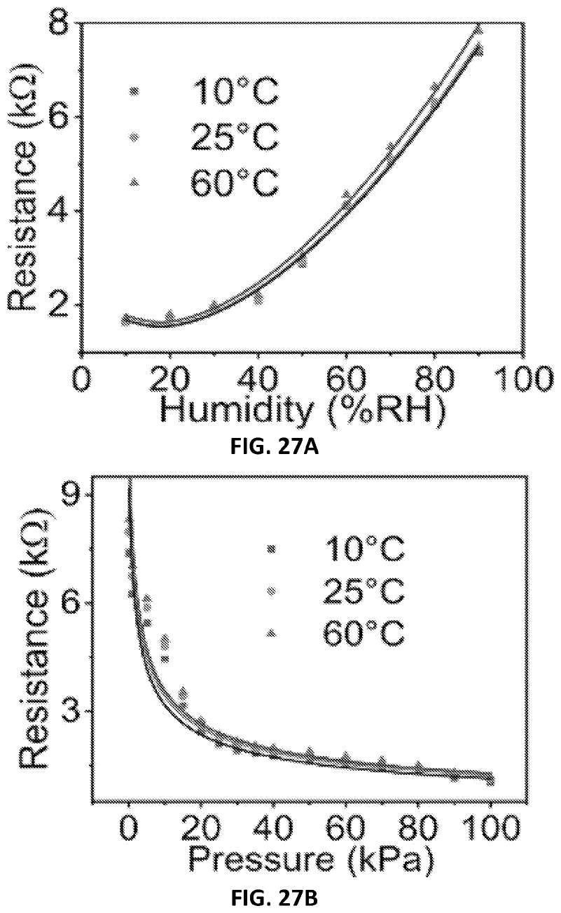

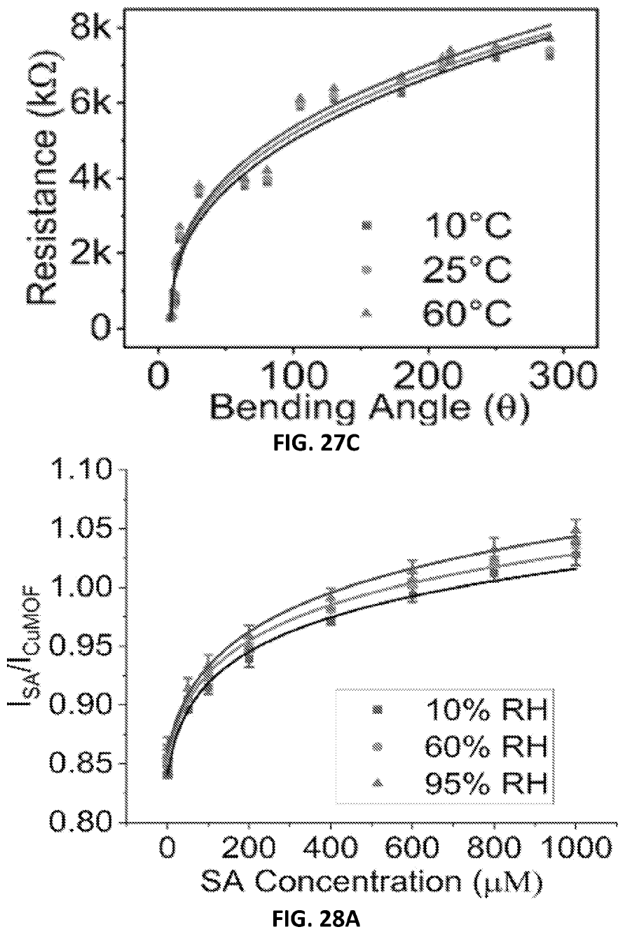

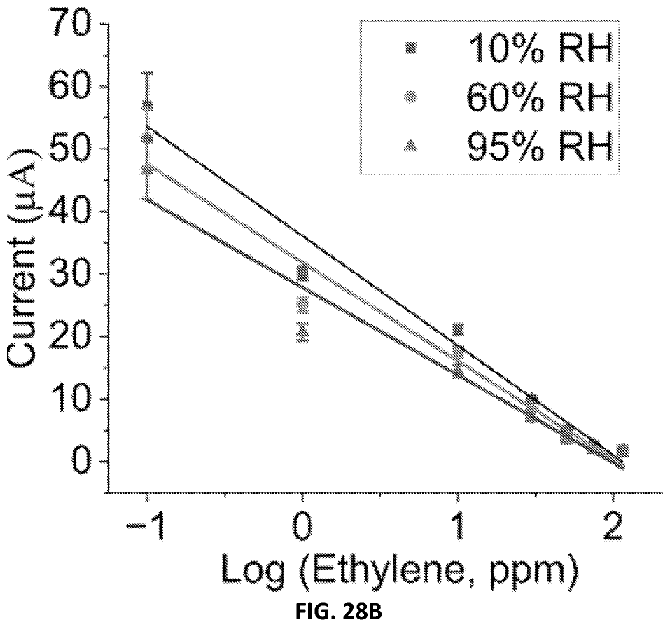

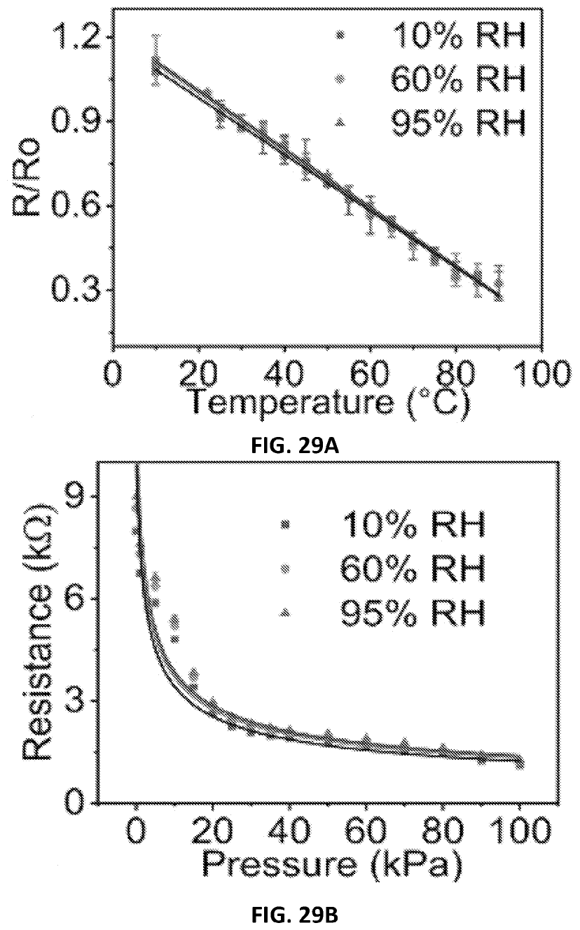

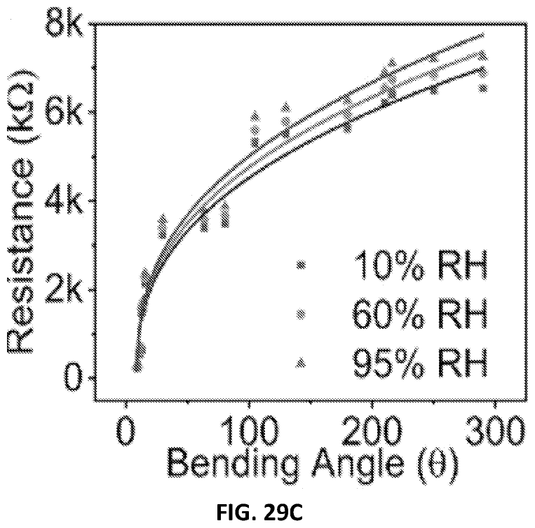

Temperature and Humidity Corrections. All but the temperature sensor was tested and corrected for temperature variations (since the temperature sensor was made to respond to temperature variations). FIGS. 26A-26B and FIGS. 27A-27C show the calibration plots of the sensors under varying temperature conditions. Likewise, FIGS. 28A-28B and FIGS. 29A-29C show the responses of all but the humidity sensor under varying relative humidity levels (since the humidity sensor was made to respond to humidity variations). The coefficient of variance between the calibration plots was found to be less than 9%.

The correction factors for the intercept, slope, and exponent (in case of nonlinear power fit) calculated at different temperature, humidity, and bending were found using the following equations.

$${f}_{intercept ( temp} = \frac{intercept ( temp )}{intercept ( 25 ° C . )} \qquad ( 8 )$$

$${f}_{i n t e r cept ( \% RH )} = \frac{intercept ( \% RH )}{intercept ( \%60 )} \qquad ( 9 )$$

$${f}_{intercept ( bend )} = \frac{intercept ( bending )}{intercept ( no bending )} \qquad ( 10 )$$

$${f}_{slope ( temp} = \frac{slope ( temp )}{slope ( 25 ° C . )} \qquad ( 11 )$$

$${f}_{slope ( \% RH )} = \frac{slope ( \% RH )}{slope ( \%60 )} \qquad ( 12 )$$

$${f}_{slope ( bend )} = \frac{slope ( bending )}{slope ( no bending )} \qquad ( 13 )$$

$${f}_{exponent ( temp )} = \frac{exponent ( temp )}{exponent ( 25 ° C . )} \qquad ( 14 )$$

$${f}_{exponent ( \% RH )} = \frac{exponent ( \% RH )}{exponent ( \%60 )} \qquad ( 15 )$$

$${f}_{exponent ( bend )} = \frac{exponent ( bending )}{exponent ( no bending )} \qquad ( 16 )$$

The entire response of each sensor was corrected on the basis of recalculating the slope, intercept, and exponent of the initial calibration graph. The corrected intercept, slope and exponent were found using the following equations:

$$intercept ( corr . ) = {f}_{intercept} ( temp ) {f}_{intercept} ( \% RH ) {f}_{intercept} ( bend ) intercept ( init . ) \qquad ( 17 )$$

$$slope ( corr . ) = {f}_{slope} ( temp ) {f}_{slope} ( \% RH ) {f}_{slope} ( bend ) slope ( init . ) \qquad ( 18 )$$

$$exponent ( corr . ) = {f}_{exponent} ( temp ) {f}_{exponent} ( \% RH ) {f}_{exponent} ( bend ) exponent ( init . ) \qquad ( 19 )$$

### Example 11

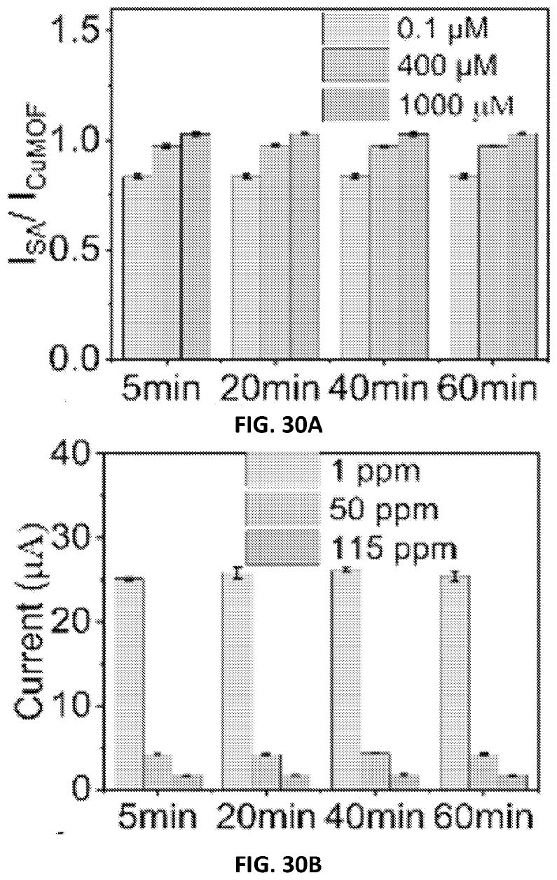

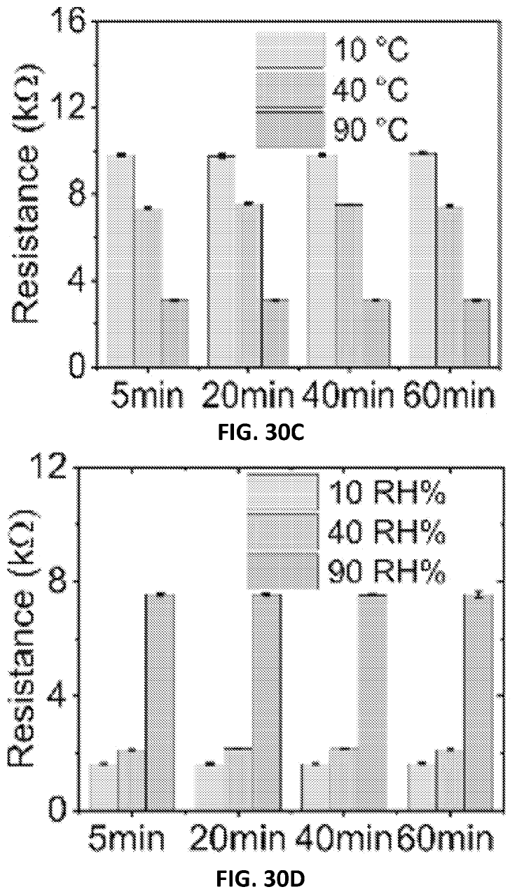

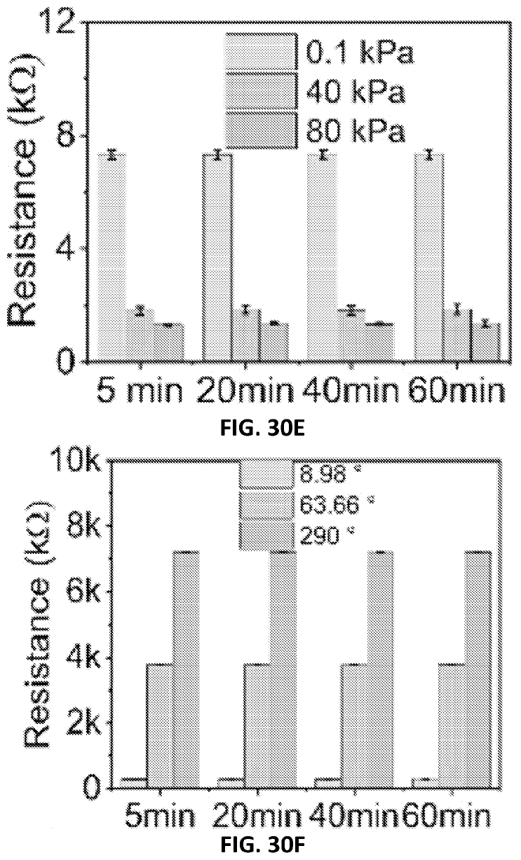

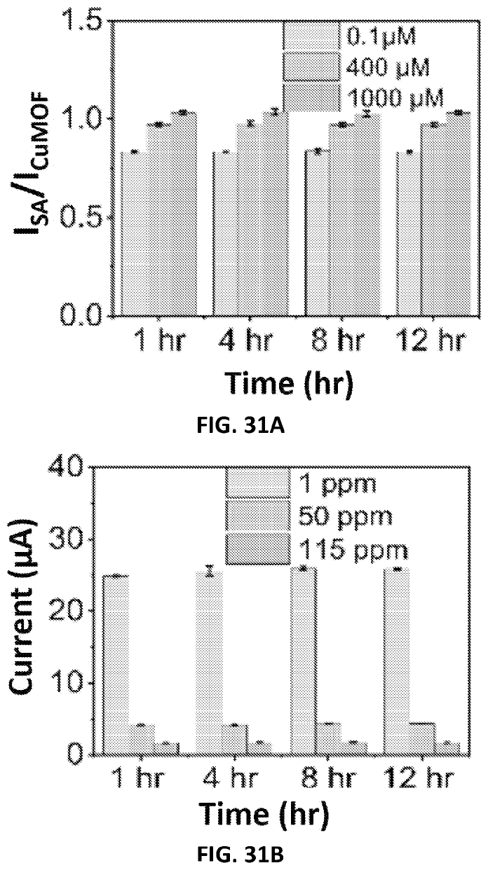

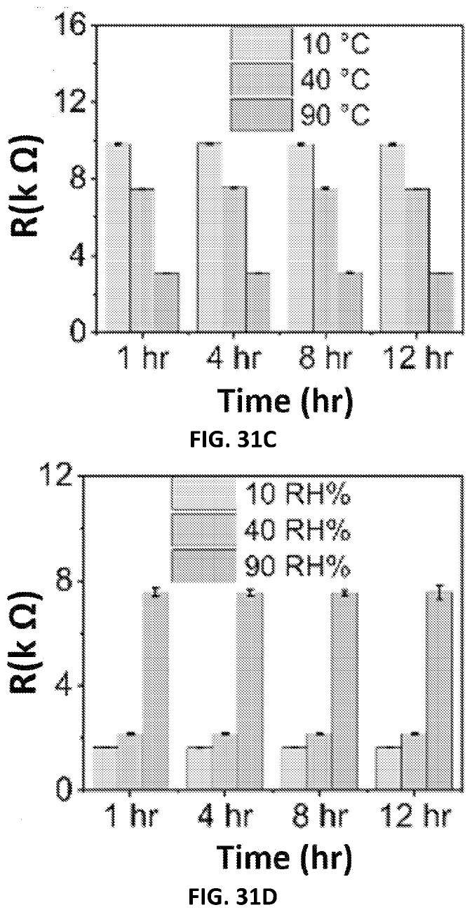

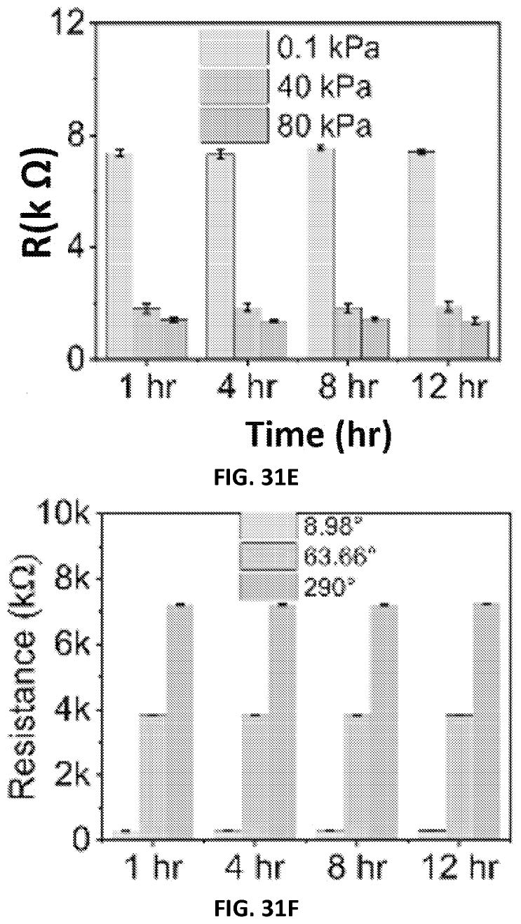

Drift Analysis. All the sensors were analyzed for drift by measuring the sensor response continuously over an hour and then every hour over a 12-hour period. The sensors were characterized for drift by first measuring the sensor response every 20 minutes over an hour (FIGS. 30A-30F, including for (FIG. 30A) salicylic acid sensor at 0.1, 400, and 1000 μM concentrations, (FIG. 30B) ethylene sensor at 1, 50, and 115 ppm, (FIG. 30C) temperature sensor at 10, 40, and 90° C., (FIG. 30D) humidity sensor at 10, 40, and 90 RH %, (FIG. 30E) pressure sensor at 0.1, 40, and 80 kPa, and (FIG. 30F) strain sensor at 8.98, 63.66, and 290° bending angles. The drift was investigated over 12 hours for (FIG. 31A) SA sensor at 0.1, 400, and 1000 μM concentrations, (FIG. 31B) ethylene sensor at 1, 50, and 115 ppm, (FIG. 31C) temperature sensor at 10, 40, and 90° C., (FIG. 31D) humidity sensor at 10, 40, and 90 RH %, (FIG. 31E) pressure sensor at 0.1, 40, and 80 kPa, and (FIG. 31F) strain sensor at 8.98, 63.66, and 290° bending angles. The mean coefficient of variance was <5% over one hour and <8% over 12 hours, indicating the minimal drift displayed by the sensors.

### Example 12

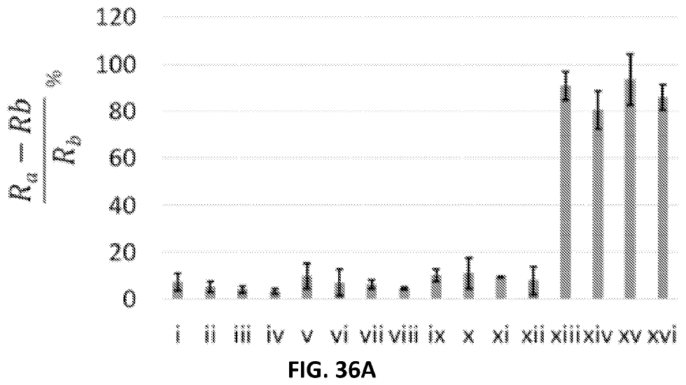

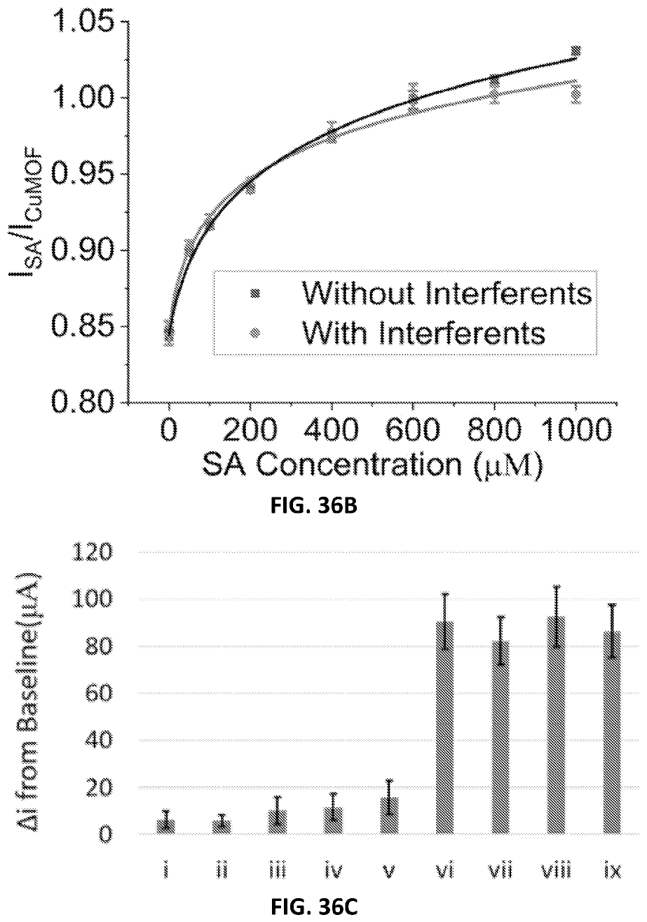

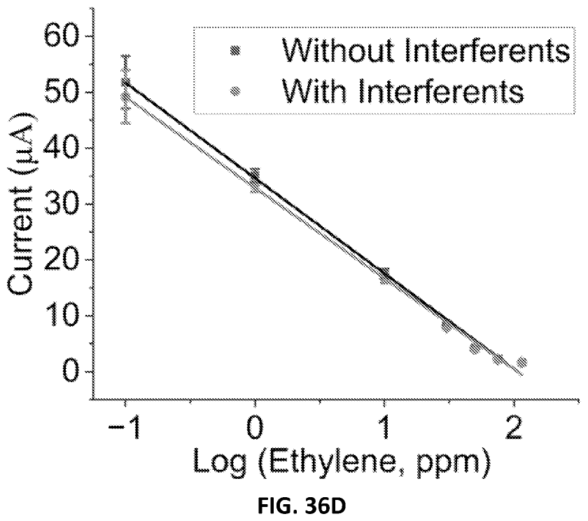

Selectivity Test. To successfully deploy the sensor for real-time monitoring of plant health parameters, it is essential to evaluate the response of the sensors to interfering species. Hence, the salicylic acid sensor was tested against other chemical compounds typically found in plant sap (FIG. 36A). The following solutions were prepared for the selectivity test: (i) 50 μM of glucose, (ii) 50 μM of sucrose, (iii) 50 μM of soluble starch, (iv) 50 μM of L-tryptophan, (v) 50 μM of L-cysteine, (vi) 50 μM of abscisic acid (ABA), (vii) 50 μM of gibberellic acid (GA), (viii) 50 μM of Jasmonic acid (JA), (ix) 50 μM of oleic acid (OA), (x) 50 μM of indole-3-acetic acid (IAA), (xi) 50 μM of citric acid (CA), (xii) 50 μM of salicylic acid (SA), (xiii) a mixture of 50 μM glucose, soluble starch, L-tryptophan, L-cysteine, ABA, GA, JA, OA, IAA, CA each, (xiv) a mixture of 50 μM glucose, soluble starch, L-tryptophan, L-cysteine, ABA, GA, JA, OA, IAA, CA each, and 100 μM of SA, (xv) 900 μM of SA, and (xvi) a mixture of 50 μM glucose, soluble starch, L-tryptophan, L-cysteine, ABA, GA, JA, OA, IAA, CA each, and 900 μM of SA. The relative signals

$$( \frac{R a - R b}{R b} ) ,$$

where Ra=ratio of hormone redox current and CuMOF current and Rb=ratio of base current and CuMOF current) for the solutions not containing SA were significantly lower compared to the solutions containing SA. Moreover, the calibration curve of the SA sensor was plotted in presence of 50 M of glucose, soluble starch, L-tryptophan, L-cysteine, ABA, GA, JA, OA, IAA, CA each. No noticeable variation was observed between the calibration curves in FIG. 20B (in the absence of any interferents) and FIG. 36B, thereby confirming excellent selectivity of the sensor.

The ethylene sensor was also tested in response to various gaseous interferents typically present in the agricultural lands or emitted from the plants. For instance, the ethylene sensor was tested against the following gases: (i) 50 ppm of nitrogen (N₂), (ii) 50 ppm of methane (CH₄), (iii) 50 ppm of nitrous oxide (N₂O), (iv) 50 ppm of ammonia (NH₃), (v) a mixture of 50 ppm of N₂, CH₄, N₂O, NH₃ each, (vi) 10 ppm of ethylene, (vii) 115 ppm ethylene, (viii) a mixture of 50 ppm of N₂, CH₄, N₂O, NH₃ each, and 10 ppm of ethylene, and (ix) a mixture of 50 ppm of N₂, CH₄, N₂O, NH₃ each, and 115 ppm of ethylene. The peak current variations from the baseline for gases other than ethylene were significantly less compared to when ethylene was present in the mixture (FIG. 36C). Gases tested include (i) 50 ppm of nitrogen (N₂), (ii) 50 ppm of methane (CH₄), (iii) 50 ppm of nitrous oxide (N₂O), (iv) 50 ppm of ammonia (NH₃), (v) a mixture of 50 ppm of N₂, CH₄, N₂O, NH₃ each, (vi) 10 ppm of ethylene, (vii) 115 ppm ethylene, (viii) a mixture of 50 ppm of N₂, CH₄, N₂O, NH₃ each, and 10 ppm of ethylene, and (ix) a mixture of 50 ppm of N₂, CH₄, N₂O, NH₃ each, and 115 ppm of ethylene. Moreover, negligible difference was observed between the ethylene sensor calibration plots in the presence (FIG. 36D) and absence (FIG. 20D) of the interfering gases.

### Example 13

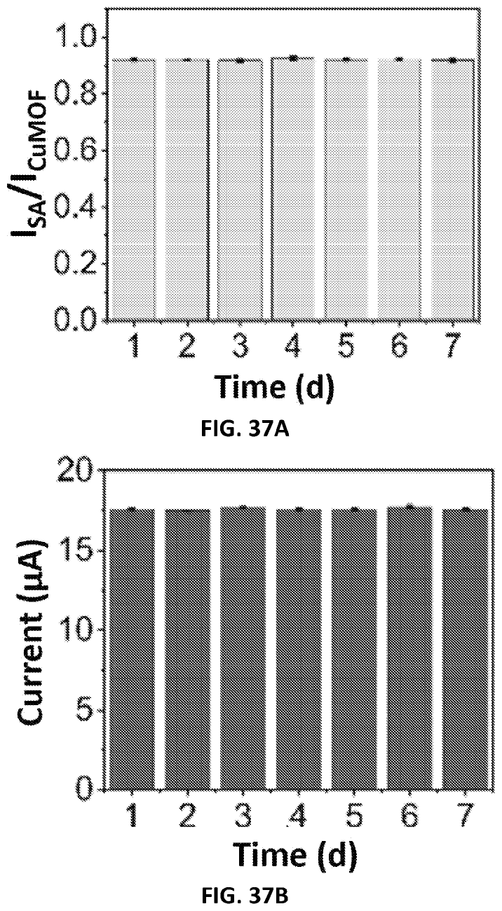

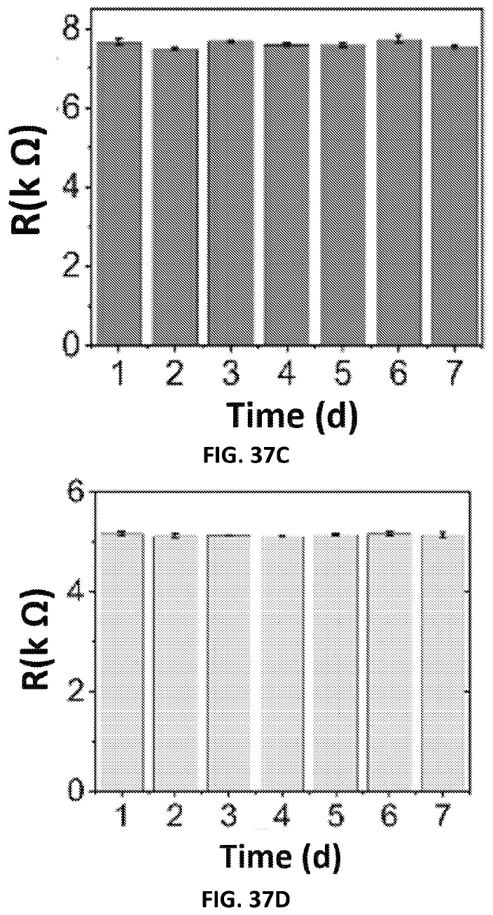

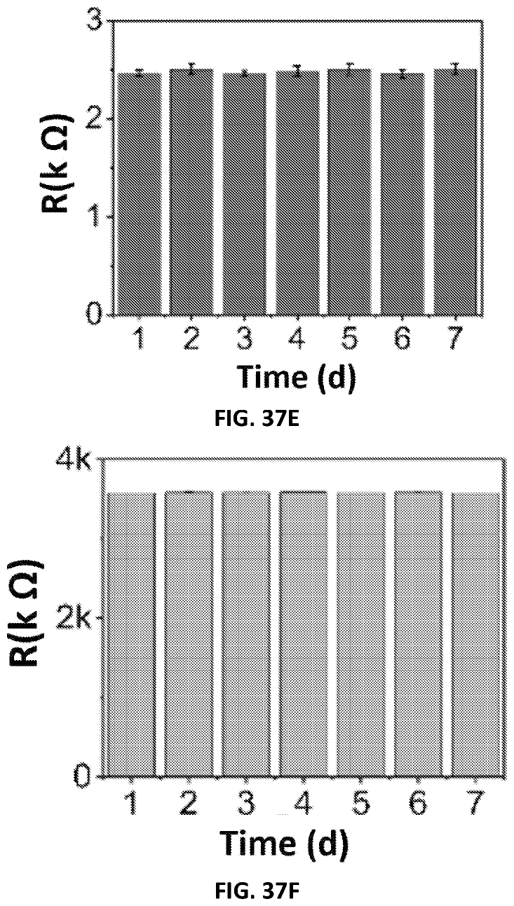

Stability Test. The long-term stability of the six sensors was evaluated by testing the sensors over a week. The sensor responses are demonstrated in FIG. 37A-37F. The long-term stability of (FIG. 37A) salicylic acid sensor (tested with 100 μM), (FIG. 37B) ethylene sensor (tested with 10 ppm), (FIG. 37C) temperature sensor (tested with 35° C.), (FIG. 37D) humidity sensor (tested with 75% RH), (FIG. 37E) pressure sensor (tested with 20 kPa), and (FIG. 37F) strain sensor (testing with 30° bending angle). The coefficient of variance in the sensor response was measured to be <2% over 7 days, indicating an acceptable stable response for on-plant measurements.

### Example 14

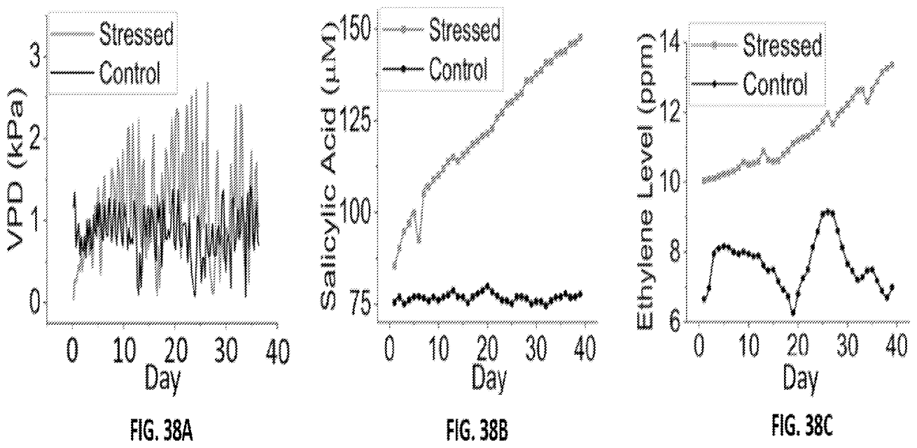

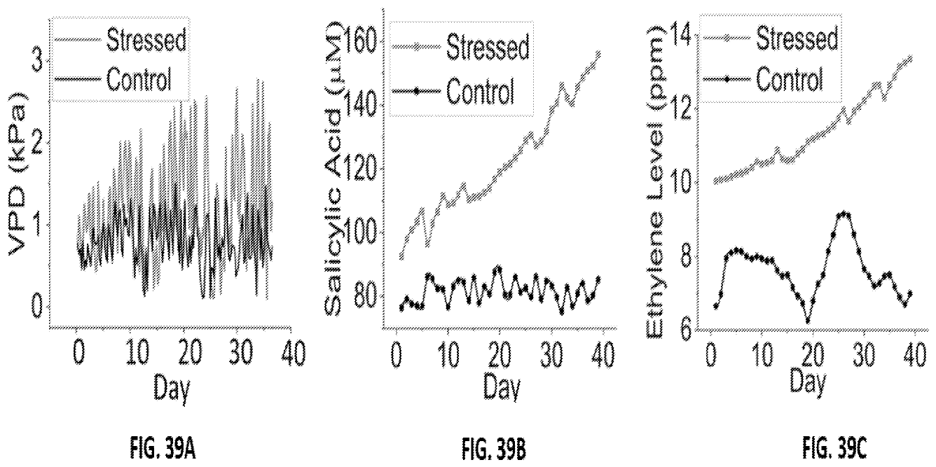

Real-time Measurements of Plant Health. SA and ethylene levels measured with the sensors were validated against the values from liquid and gas chromatography mass spectrometry measurements. The sensors were tested on live plants by placing the flexible patch carrying the temperature, humidity, SA, and ethylene sensors beneath the leaf of a bell pepper plant. The combined strain and pressure sensors were wrapped around the stem. A micron scale (˜10 μm) hole was punched on the leaf so that sap from the vascular bundle could reach the sensor surface for SA detection. Measurements were recorded from 4 bell pepper plants: one was water stressed and kept in sunlight (case I), one was a control, unstressed plant kept in sunlight (case II), one was water stressed and kept in the shade (case III) and one was a control, unstressed plant kept in the shade (case IV). Measurements were collected from all the sensors for 40 days (from September 18 to October 28). Temperature and humidity measurements were collected four times a day (9:30 am, 1:00 μm, 5:00 μm, and 8 p.m.) while the salicylic acid, ethylene, pressure, and strain sensor measurements were collected once a day (at 1:00 μm). The results are demonstrated in FIGS. 38A-38F (sunlight) and FIGS. 39A-39F (shade).

All the tests were performed at the University of Texas at Tyler (32.3163° N and 95.2510° W). The stressed plants were not irrigated during the stress period, while the control plants were irrigated with 50 mL of water every day at 9:30 am. No fertilizer or insecticides were applied. The water stressed plants exhibited elevated levels of VPD, SA and ethylene concentrations starting from day 1 of stress. FIGS. 38A-38F and FIGS. 39A-39F show the SA and ethylene levels after correcting for intercept, slope and exponents according to Equations (14)-(16). These results are significant as it would enable early diagnosis of crop stress and facilitate immediate intervention measures to reduce productivity losses.

The vapor pressure deficit (VPD) was measured using the following equations:

$$VPD = VPsat - VPair \qquad ( 20 )$$

$$VPsat = 0.6107 \times {10}^{\frac{7.5 T 1}{237.3 + T 1}} \qquad ( 21 )$$

$$VPsat = 0.6107 \times {10}^{\frac{7.5 Ta}{237.3 + T a}} \times \frac{R H}{100} \qquad ( 22 )$$

Where VPsat=plant's saturated vapor pressure (psi) and VPair=vapor pressure of the air.
   T₁ and Tₐ represent temperatures on the leaf surface and in air (in ° C.).

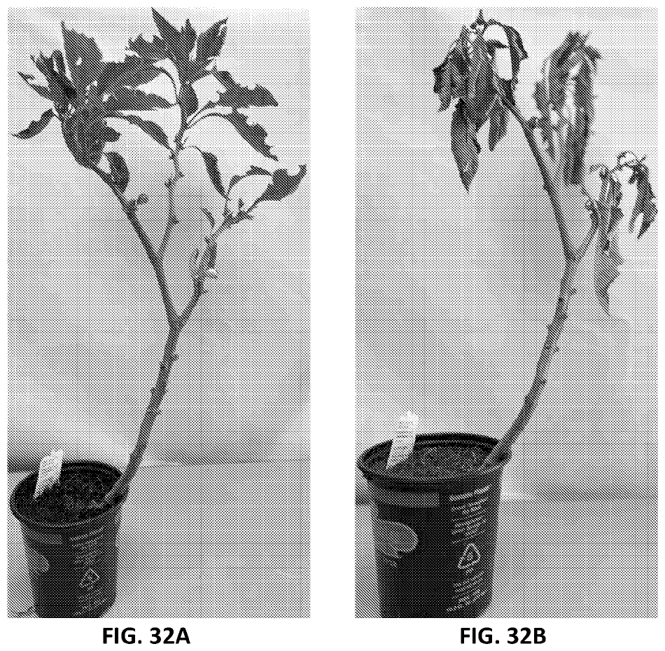

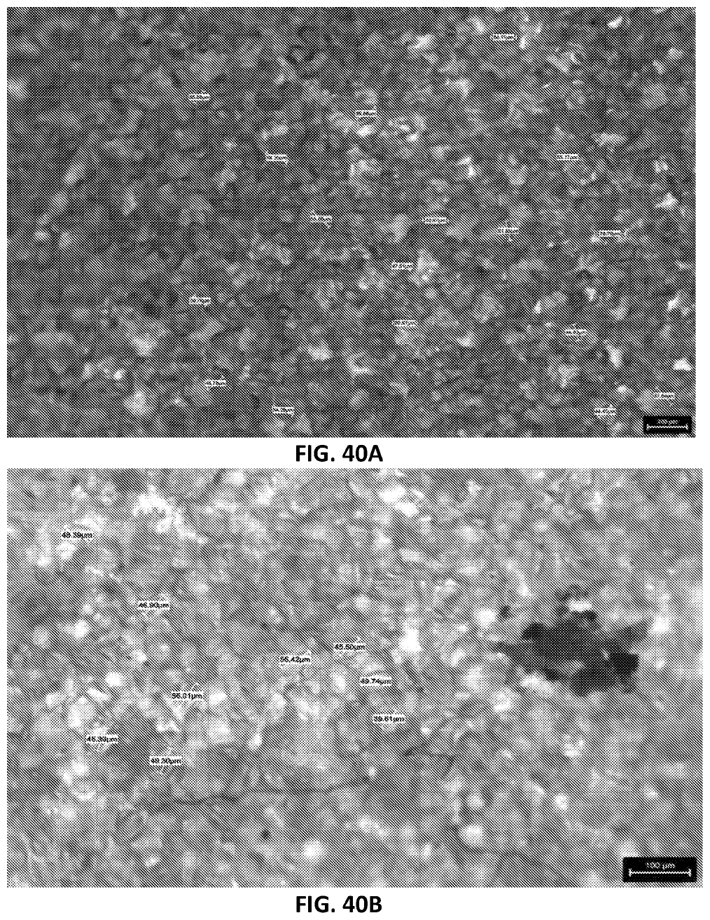

The variations in leaf temperature, humidity, and VPD levels were found to coincide with the expectation. The leaf temperature (in both stressed and control plants) was always lower compared to the air temperature, which was due to the cooling effects of transpiration. In addition, the relative humidity beneath the leaf was found to be higher than the relative humidity of the open air, which was a driving factor for transpiration. The VPD values were higher in the water stressed plants as compared to the control plants, indicating the closure of stomata and hence reluctance of the plant to lose water. The microscopic images of water stressed plants confirmed this stomata closure (FIGS. 40A-40B). FIGS. 32A-32B show the optical images of water-stressed (FIG. 32B) and unstressed (FIG. 32A) bell pepper plants taken after 40 days of measurements. Also, the salicylic acid and ethylene levels were elevated in both water-stressed plants kept under sunlight and in the shade. The rise in the SA and ethylene levels was faster than the VPD. Moreover, VPD levels in the water stressed plant under sunlight increased faster than those in the water stressed plant in the shade. The stem diameter for both the water stressed plants started to decrease owing to the lack of water as compared to the unstressed plats (Table V).

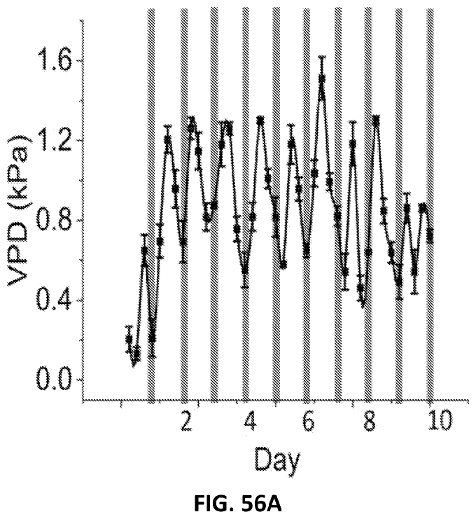

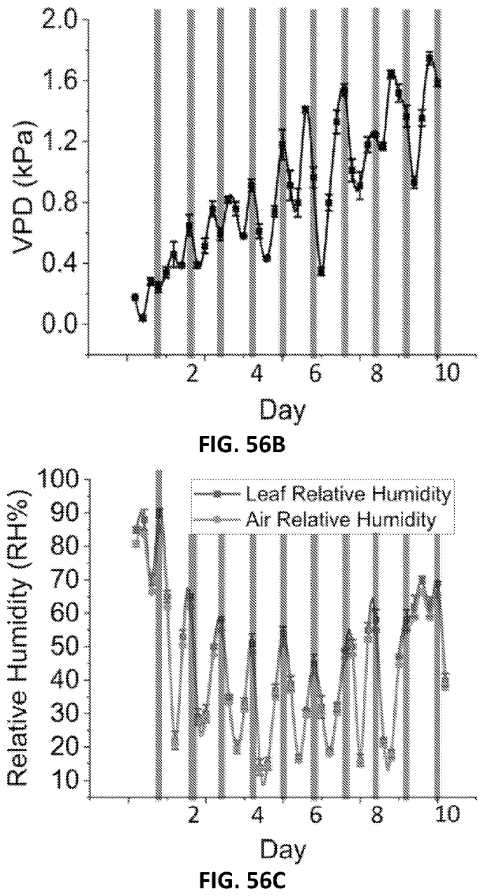

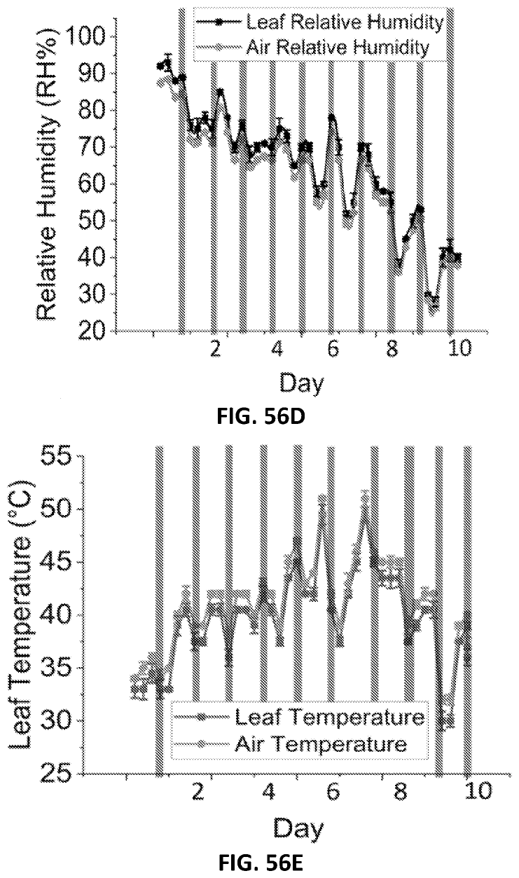

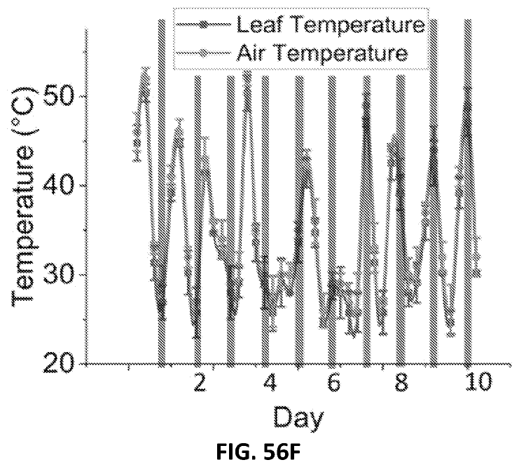

The real-time VPD, leaf RH, and leaf temperature measurements recorded over 10 days are shown in FIGS. 56A-56F. VPD is an effective measure of the transportation of water from root to shoot. Increased VPD leads to rapid transpiration in plants, which results in over-drying and stressing of the plants. In contrast, a lower VPD value indicates vapor saturation on the leaf surface, which can lead to fungal infection in leaves. Hence, it is crucial to maintain an optimum VPD level in plants. VPD values measured with temperature and humidity sensors were found to be higher in the water-stressed plants as compared to the control plants, indicating the closure of stomata and hence the reluctance of the plant to transpire. It is noteworthy that stomatal closure is a common adaptation response of plants to the onset of drought. VPD levels in the water-stressed plant kept in the sunlight (FIG. 38A) increased faster than the levels measured in the water-stressed plant kept in the shade (FIG. 39A). This finding suggests that the plants continuously exposed to the sun encountered additional heat stress, resulting in an elevated level of VPD. Moreover, a progressive increase in SA and ethylene levels was observed in water-stressed plants starting from Day 1 (FIGS. 38B-38C and FIGS. 39B-39C). The SA levels in the control plants fluctuated around a mean of 75.35 μM with a standard deviation of 2.523 μM in FIG. 38B and a mean of 80.31 μM with a standard deviation of 3.226 μM in FIG. 39B. Likewise, the ethylene levels in the control plants fluctuated around a mean of 8.81 ppm with a minimal standard deviation of 0.311 ppm in FIG. 38C and a mean of 10.21 ppm with a standard deviation of 0.336 ppm in FIG. 39C. Therefore, the sensor demonstrated its ability to distinguish hormone emissions between stressed and unstressed plants. These findings enable early diagnosis of crop stress from SA and ethylene levels and facilitate immediate intervention measures to reduce stress-induced productivity losses. The radial growth (derived from stem diameter) of both the water-stressed plants went down owing to water deficiency and hence the crop growth declined (see Table VII, below).

TABLE VII

| | unstressed | unstressed | Stressed | Stressed |
|---|---|---|---|---|
| | (in sunlight) | (in shade) | (in sunlight) | (in shade) |
| Day | Diameter (mm) | Diameter (mm) | Diameter (mm) | Diameter (mm) |
| 1 | 6.49 | 6.17 | 6.94 | 6.59 |
| 2 | 6.49 | 6.17 | 6.94 | 6.59 |
| 3 | 6.5 | 6.18 | 6.94 | 6.59 |
| 4 | 6.52 | 6.19 | 6.92 | 6.57 |
| 5 | 6.52 | 6.19 | 6.91 | 6.56 |
| 6 | 6.53 | 6.2 | 6.9 | 6.56 |
| 7 | 6.54 | 6.21 | 6.88 | 6.54 |
| 8 | 6.57 | 6.24 | 6.87 | 6.53 |
| 9 | 6.6 | 6.27 | 6.86 | 6.52 |
| 10 | 6.61 | 6.28 | 6.85 | 6.51 |
| 11 | 6.64 | 6.31 | 6.82 | 6.48 |
| 12 | 6.65 | 6.32 | 6.82 | 6.48 |
| 13 | 6.68 | 6.35 | 6.82 | 6.48 |
| 14 | 6.68 | 6.35 | 6.81 | 6.47 |
| 15 | 6.68 | 6.35 | 6.8 | 6.46 |
| 16 | 6.68 | 6.35 | 6.79 | 6.45 |
| 17 | 6.69 | 6.36 | 6.78 | 6.44 |
| 18 | 6.69 | 6.36 | 6.77 | 6.43 |
| 19 | 6.7 | 6.37 | 6.77 | 6.43 |
| 20 | 6.7 | 6.37 | 6.77 | 6.43 |
| 21 | 6.7 | 6.37 | 6.76 | 6.42 |
| 22 | 6.71 | 6.37 | 6.76 | 6.42 |
| 23 | 6.72 | 6.38 | 6.75 | 6.41 |
| 24 | 6.73 | 6.39 | 6.75 | 6.41 |
| 25 | 6.74 | 6.4 | 6.75 | 6.41 |
| 26 | 6.75 | 6.41 | 6.75 | 6.41 |
| 27 | 6.75 | 6.41 | 6.72 | 6.38 |
| 28 | 6.75 | 6.41 | 6.71 | 6.37 |
| 29 | 6.78 | 6.44 | 6.68 | 6.35 |
| 30 | 6.79 | 6.45 | 6.67 | 6.34 |
| 31 | 6.8 | 6.46 | 6.64 | 6.31 |
| 32 | 6.81 | 6.47 | 6.61 | 6.28 |
| 33 | 6.83 | 6.49 | 6.6 | 6.27 |
| 34 | 6.84 | 6.5 | 6.59 | 6.26 |
| 35 | 6.85 | 6.51 | 6.59 | 6.26 |
| 36 | 6.87 | 6.53 | 6.57 | 6.24 |
| 37 | 6.87 | 6.53 | 6.55 | 6.22 |
| 38 | 6.87 | 6.53 | 6.55 | 6.22 |
| 39 | 6.87 | 6.53 | 6.55 | 6.22 |
| 40 | 6.87 | 6.53 | 6.54 | 6.21 |

### Example 15

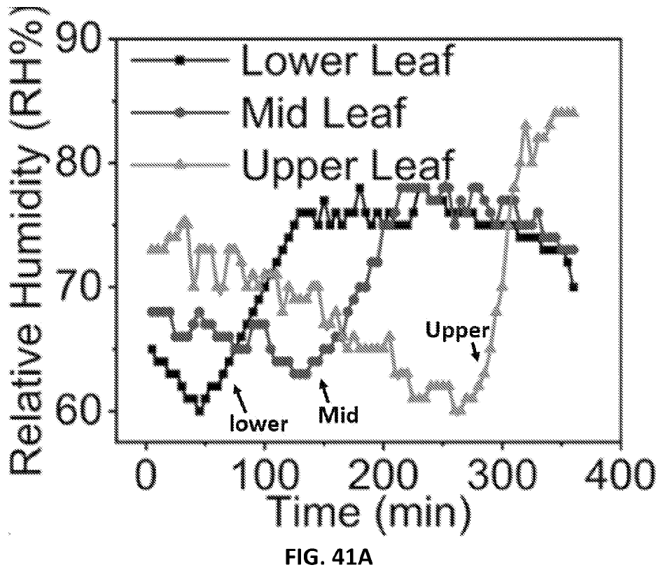

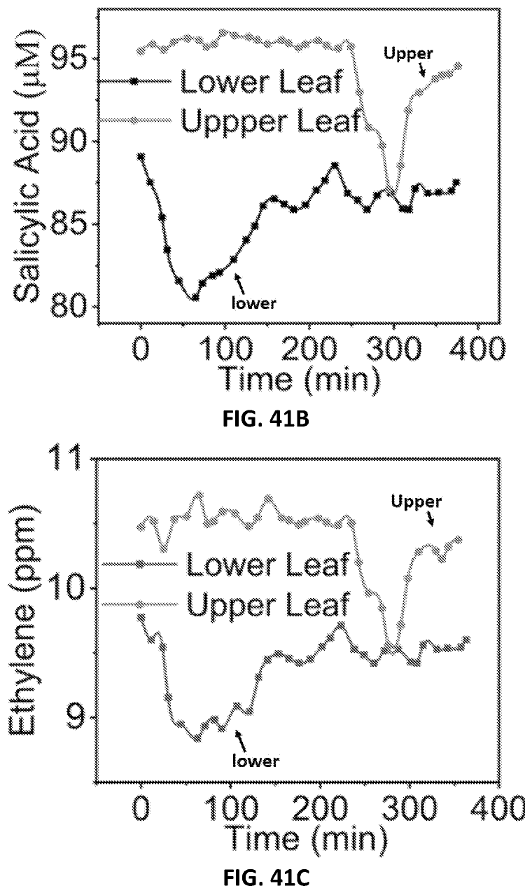

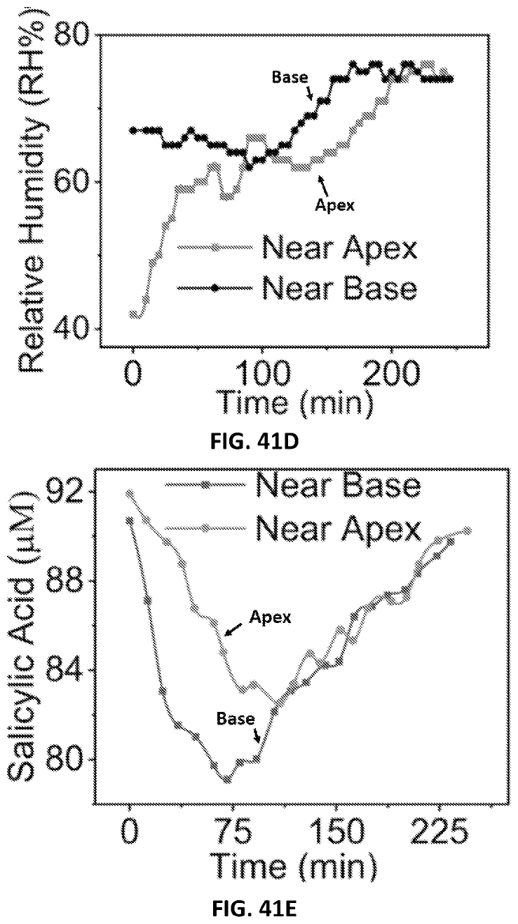

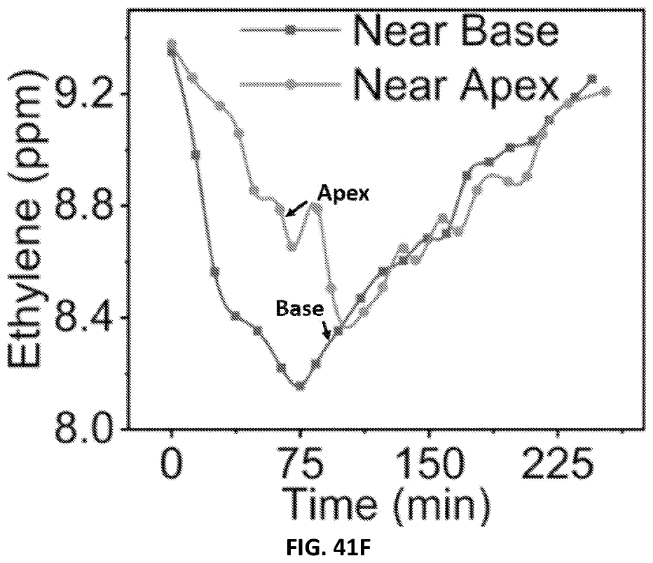

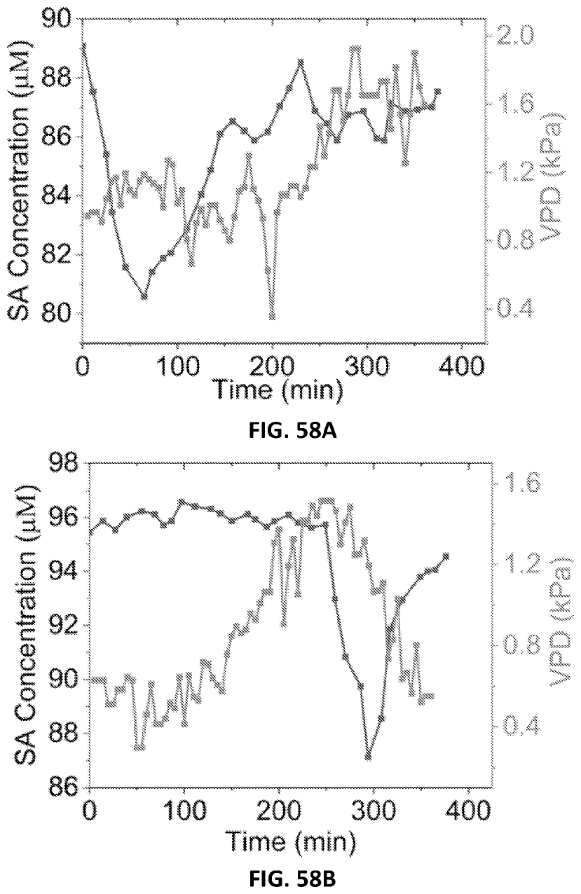

Kinetics of SA and Ethylene Transport across Bell Pepper Plants. The sensor suite was used to monitor water transport across a bell pepper plant (FIGS. 41A-41F). A correlation was observed between the hormone levels (i.e., SA and ethylene) and water transport (FIGS. 41A-41F). Three sensors were installed at the lower (40 cm), middle (75 cm), and upper (105 cm) leaves, with the leaf height measured from the soil surface. The plant was irrigated before the test. The transition in RH levels at the leaves located at three different heights indicate water transport from the root to the shoot. The kinetics of SA and ethylene levels were found to highly correlate with the kinetics of water transport (represented by leaf RH and VPD values). An upward transition in leaf RH was followed by a downward transition in SA and ethylene levels almost instantaneously. A decrease in the SA and ethylene levels was most likely due to water reaching the leaf. It took almost 221 minutes for the water to reach from lower to upper leaves. FIGS. 58A-58B further depict a comparative analysis of the kinetics of SA and VPD levels at the lower (FIG. 58A) and upper (FIG. 58B) leaves. The results further confirm the correlation between SA and VPD kinetics. Over 400 minutes, except for the transitions, the upper leaf emitted a greater amount of SA and ethylene (96.36±4.35 μM for SA and 10.8±0.22 ppm for ethylene) than the lower leaf (87.11±3.5 μM for SA and 9.68±0.34 ppm for ethylene), as illustrated in FIGS. 41A-41F. These results demonstrate the relationship between sensor-measured phytohormone levels and soil water availability. Moreover, the spatiotemporal distribution of water and hormone levels across the whole plant can be investigated by mounting sensors at multiple locations of the same plant. The results demonstrate that the sensors provide a means of improved productivity and high water-use efficiency under drought conditions.

Furthermore, the difference in SA and ethylene levels between the base (near the stem) and apex (tip of the leaf) of the same leaf (located 75 cm above the soil surface) was also measured, as shown in FIGS. 41D-41F. For this experiment, two sensors were installed 5 cm apart on the same leaf. The differences in the RH, SA, and ethylene dynamics were observed at the leaf scale.

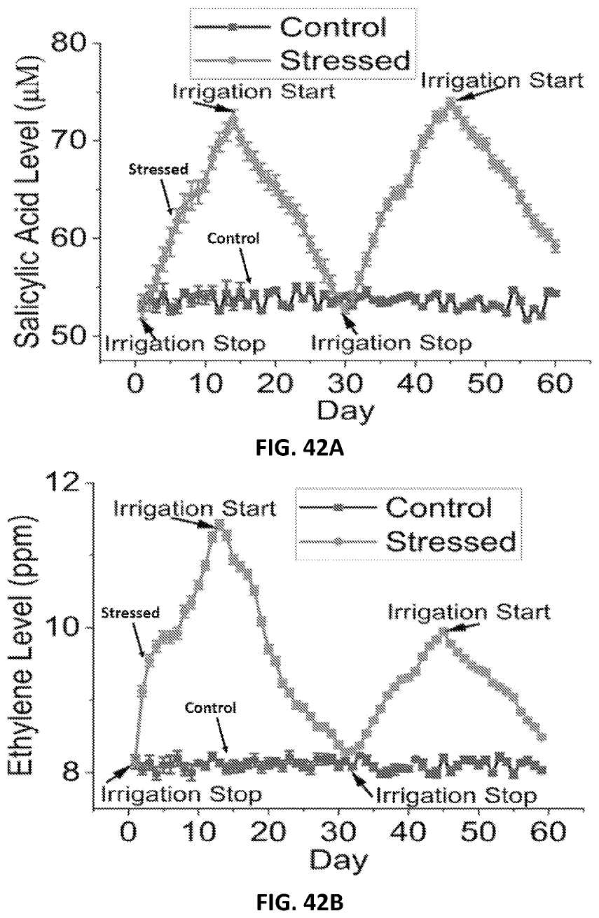

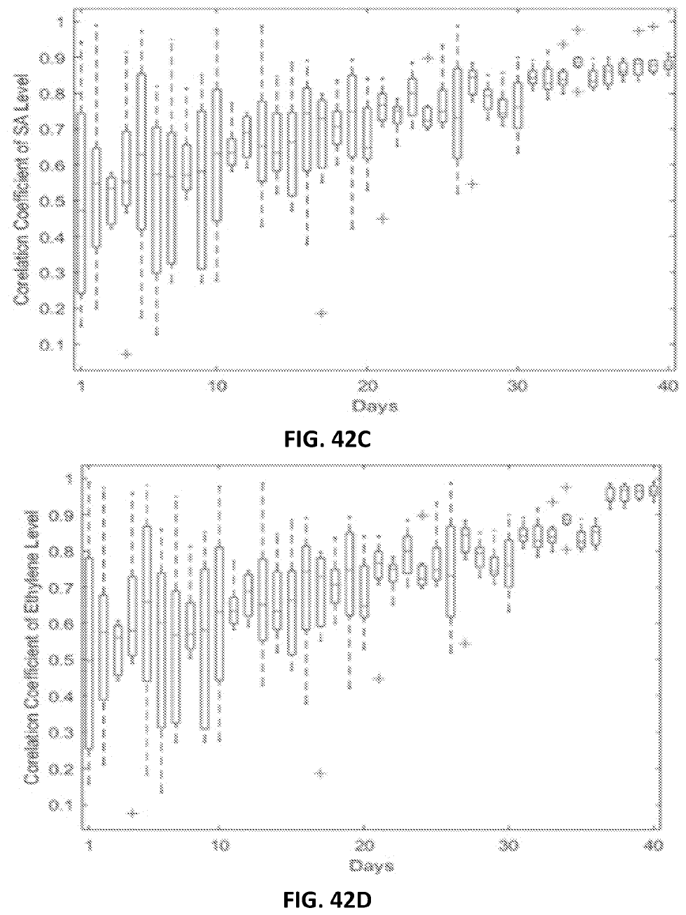

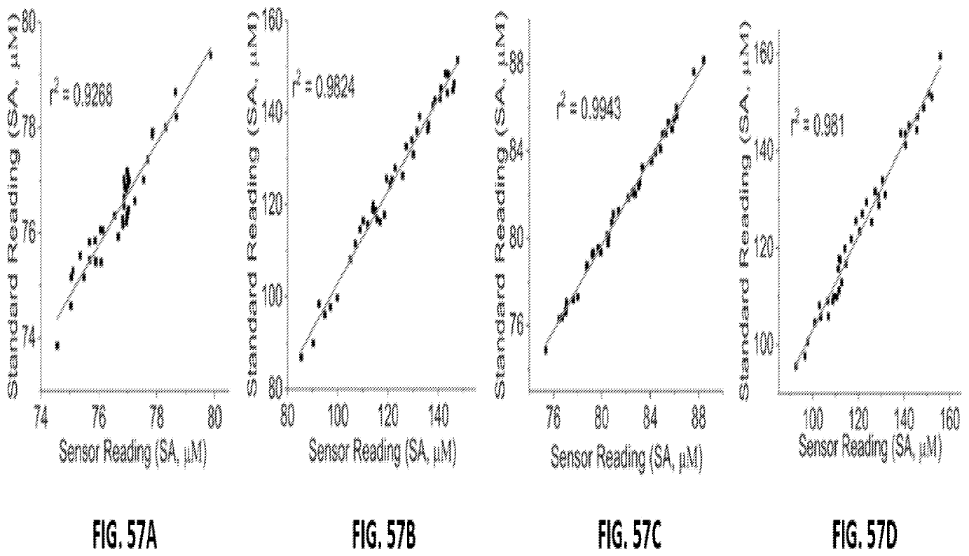

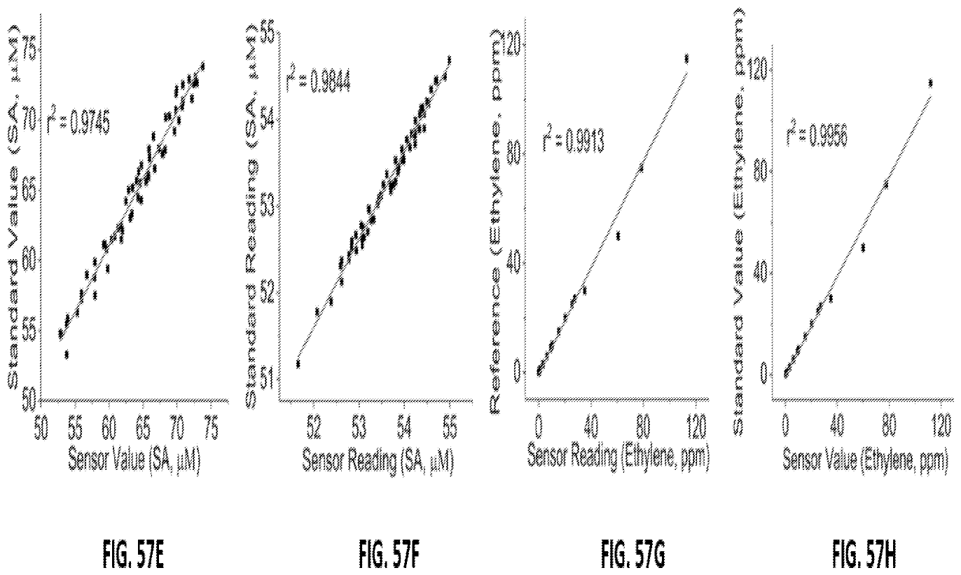

Cyclic Water Stress Experiments in Cabbage Plants. The same tests were repeated with small cabbage plants. The sensors were demonstrated to measure SA and ethylene levels in cabbage plants under periodic water stress conditions over 2 months (from September to November). Ten cabbage plants were subjected to water stress and ten plants were unstressed (control). The stressed plants were not irrigated during the stress period, while the control plants were irrigated with 20 mL of water every day. Two cycles of water stress were applied wherein each cycle lasted 30 days. Water stress was applied in the first 15 days, followed by irrigation in the next 15 days. This 30-day cycle was repeated. SA and ethylene levels were measured once a day (at 1:00 μm). A noticeable time-series correlation was observed between the SA/ethylene levels and the water stress periods (FIGS. 42A-42B). The hormone levels started to elevate in response to a water stress condition, while immediately after irrigation the hormone levels declined. The time-series SA levels measured with our sensor in the leaves of live cabbage plants over 60 days were validated against the values from high-performance liquid chromatography, as shown in FIGS. 57E-57F. It should be noted that although all the plants were grown under the same environmental conditions, plant-to-plant variations in growth, development, and metabolism were observed. Therefore, a statistical analysis was performed based on the hormone levels obtained from multiple plants. The SA and ethylene levels in the 10 water-stressed plants were analyzed via autocorrelation analysis, as shown in FIGS. 42C-42D Although the autocorrelation coefficient demonstrated a large standard deviation during the first 10 days of the experiment, the deviation started to diminish eventually and reached ±0.910589 for SA and ±0.98633 for ethylene. This could perhaps be explained by the plant-to-plant variability in hormonal responses at the beginning. However, as days passed, the plants got acclimated to the water stress condition and exhibited a similar phytohormone response with a smaller standard deviation. As a result, the optimum number of sensors to be deployed per agricultural field can be determined. For examples, in some embodiments one sensor may be deployed per acre of field wherein crops exhibit similar phytohormone responses driven by micro-environmental factors or soil structure/treatment.

Time-series of measured SA levels. Time-series SA levels measured with sensor in the leaves of live bell pepper plants over 40 days were validated against the values from high-performance liquid chromatography, as shown in FIGS. 57A-57H. A few microliters of sap samples were collected from the plants every day over the 40 days. The samples were analyzed with high-performance liquid chromatography equipment to measure the SA concentrations. The sensor could accurately estimate the SA concentrations in both control and water-stressed plants over 40 days. A high Pearson correlation coefficient greater than 0.92 was observed, suggesting the excellent reliability of the sensor. An indirect approach was used to analyze the ethylene gas directly emitted from plant leaves. First, the ethylene sensor was calibrated for a known set of ethylene concentrations mixed with other interfering gases (i.e., N₂, CH₄, N₂O, and NH₃). Next, the sensor responses were recorded for unknown ethylene concentrations emitted from the plant. Afterward, the sensor was exposed to a range of known ethylene concentrations to find out the concentrations at which the sensor response approximately matched with the responses recorded for unknown ethylene emitted from plants. The results were plotted in FIGS. 57G-57H, which show that our ethylene sensor has a very high accuracy, with a Pearson correlation coefficient of 0.99. Moreover, the VPD values measured with our flexible temperature and relative humidity sensors were compared against the VPD values measured with commercially available rigid temperature (LM35, Texas Instruments, TX) and humidity (DHT11, Adafruit, NY) sensors. As outlined in Tables IX and X, the sensors show excellent accuracy with minimal deviation from the commercial sensors. It was observed that compared to the traditional mass spectroscopy-based technique that requires expensive instrumentation, a disruptive and complex sampling process, and skilled operators, the sensor suite allowed real-time and in situ monitoring along with an early diagnosis of stress conditions in live plants. Moreover, in contrast to commercially available rigid integrated circuits, the sensors are flexible and easily compliant to delicate parts of the plant including leaves and stem.

TABLE IX
VPD of Plants kept in the Sunlight

| | VPD in | VPD in | | VPD in |
|---|---|---|---|---|
| | kPa with | kPa with | VPD in kPa | kPa with |
| | our | commercial | with our | commercial |
| | sensors | sensors | sensors | sensors |
| | (stressed | (stressed | (control | (control |
| Hour | plant) | plant) | plant) | plant) |
| 12 | 0.04081 | 0.040402 | 1.16426 | 1.152617 |
| 24 | 0.28002 | 0.27722 | 1.34812 | 1.334639 |
| 36 | 0.24321 | 0.240778 | 0.68033 | 0.673527 |
| 48 | 0.33989 | 0.336491 | 0.85107 | 0.842559 |
| 60 | 0.45781 | 0.453232 | 0.95876 | 0.949172 |
| 72 | 0.38812 | 0.384239 | 0.58601 | 0.58015 |
| 84 | 0.64913 | 0.642639 | 0.7987 | 0.790713 |
| 96 | 0.38948 | 0.385585 | 0.63471 | 0.628363 |
| 108 | 0.51421 | 0.509068 | 0.63864 | 0.632254 |
| 120 | 0.75737 | 0.749796 | 0.81893 | 0.810741 |
| 132 | 0.59086 | 0.584951 | 0.55071 | 0.545203 |
| 144 | 0.81817 | 0.809988 | 1.01109 | 1.000979 |
| 156 | 0.75737 | 0.749796 | 0.74774 | 0.740263 |
| 168 | 0.57939 | 0.573596 | 1.0105 | 1.000395 |
| 180 | 0.91186 | 0.902741 | 0.64663 | 0.640164 |
| 192 | 0.43272 | 0.428393 | 0.70106 | 0.694049 |
| 204 | 0.74088 | 0.733471 | 1.06413 | 1.053489 |
| 216 | 1.1796 | 1.167804 | 0.90935 | 0.900257 |
| 228 | 0.91186 | 0.902741 | 0.82068 | 0.812473 |
| 240 | 0.7987 | 0.790713 | 1.20236 | 1.190336 |
| 252 | 1.40924 | 1.395148 | 0.93562 | 0.926264 |
| 264 | 0.96434 | 0.954697 | 1.21331 | 1.201177 |
| 276 | 0.34617 | 0.342708 | 1.00643 | 0.996366 |
| 288 | 0.7987 | 0.790713 | 0.77384 | 0.766102 |
| 300 | 1.3292 | 1.315908 | 0.76809 | 0.760409 |
| 312 | 1.53938 | 1.523986 | 1.18792 | 1.176041 |
| 324 | 1.01109 | 1.000979 | 0.92666 | 0.917393 |
| 336 | 0.90939 | 0.900296 | 0.81673 | 0.808563 |
| 348 | 1.17902 | 1.16723 | 0.81716 | 0.808988 |
| 360 | 1.24642 | 1.233956 | 1.27052 | 1.257815 |
| 372 | 1.16843 | 1.156746 | 1.06466 | 1.054013 |
| 384 | 1.64114 | 1.624729 | 0.74672 | 0.739253 |
| 396 | 1.51726 | 1.502087 | 1.03386 | 1.023521 |
| 408 | 1.36528 | 1.351627 | 1.25912 | 1.246529 |
| 420 | 0.93185 | 0.922532 | 0.84885 | 0.840362 |
| 432 | 1.35444 | 1.340896 | 0.70106 | 0.694049 |
| 444 | 1.74544 | 1.727986 | 0.95811 | 0.948529 |
| 456 | 1.58344 | 1.567606 | 1.22043 | 1.208226 |
| 468 | 0.99493 | 0.984981 | 0.9445 | 0.935055 |
| 480 | 0.83091 | 0.822601 | 0.82124 | 0.813028 |
| 492 | 1.85308 | 1.834549 | 0.68908 | 0.682189 |
| 504 | 1.0909 | 1.079991 | 1.20574 | 1.193683 |
| 516 | 1.05008 | 1.039579 | 1.06466 | 1.054013 |
| 528 | 1.0909 | 1.079991 | 0.95207 | 0.942549 |
| 540 | 2.11424 | 2.093098 | 0.64913 | 0.642639 |
| 552 | 2.13706 | 2.115689 | 0.90935 | 0.900257 |
| 564 | 0.9334 | 0.924066 | 0.71793 | 0.710751 |
| 576 | 1.16368 | 1.152043 | 0.77895 | 0.771161 |
| 588 | 2.17331 | 2.151577 | 0.71793 | 0.710751 |
| 600 | 1.69142 | 1.674506 | 1.24262 | 1.230194 |
| 612 | 1.24632 | 1.233857 | 1.06286 | 1.052231 |
| 624 | 0.73503 | 0.72768 | 0.77895 | 0.771161 |
| 636 | 1.43894 | 1.424551 | 0.18082 | 0.179012 |
| 648 | 2.23919 | 2.216798 | 0.09314 | 0.092209 |
| 660 | 0.95811 | 0.948529 | 0.59816 | 0.592178 |
| 672 | 0.68908 | 0.682189 | 0.18668 | 0.184813 |
| 684 | 1.64381 | 1.627372 | 0.61842 | 0.612236 |
| 696 | 1.73809 | 1.720709 | 1.18062 | 1.168814 |
| 708 | 0.80781 | 0.799732 | 1.0105 | 1.000395 |
| 720 | 0.54052 | 0.535115 | 0.81673 | 0.808563 |
| 732 | 1.03093 | 1.020621 | 1.12133 | 1.110117 |
| 744 | 0.66023 | 0.653628 | 1.17902 | 1.16723 |
| 756 | 0.77895 | 0.771161 | 0.80428 | 0.796237 |
| 768 | 0.74678 | 0.739312 | 1.14973 | 1.138233 |
| 780 | 1.65532 | 1.638767 | 1.12042 | 1.109216 |
| 792 | 2.04416 | 2.023718 | 0.93474 | 0.925393 |
| 804 | 0.77895 | 0.771161 | 0.61293 | 0.606801 |
| 816 | 0.22749 | 0.225215 | 0.51337 | 0.508236 |
| 828 | 0.09314 | 0.092209 | 1.30793 | 1.294851 |
| 840 | 0.68029 | 0.673487 | 0.98436 | 0.974516 |
| 852 | 0.18668 | 0.184813 | 1.21222 | 1.200098 |
| 864 | 0.61842 | 0.612236 | 0.78782 | 0.779942 |
| 876 | 1.57314 | 1.557409 | 0.43587 | 0.431511 |
| 888 | 1.31383 | 1.300692 | 1.24202 | 1.2296 |
| 900 | 0.74086 | 0.733451 | 1.02279 | 1.012562 |
| 912 | 1.86773 | 1.849053 | 0.78782 | 0.779942 |
| 924 | 1.75197 | 1.73445 | 0.77563 | 0.767874 |
| 936 | 1.47095 | 1.456241 | 0.8887 | 0.879813 |
| 948 | 1.09498 | 1.08403 | 0.7881 | 0.780219 |
| 960 | 1.69663 | 1.679664 | 0.91092 | 0.901811 |
| 972 | 2.24093 | 2.218521 | 1.21331 | 1.201177 |
| 984 | 1.85994 | 1.841341 | 1.37517 | 1.361418 |
| 996 | 1.05008 | 1.039579 | 0.71793 | 0.710751 |
| 1008 | 2.24093 | 2.218521 | 0.64913 | 0.642639 |
| 1020 | 2.35817 | 2.334588 | 1.01736 | 1.007186 |
| 1032 | 2.30645 | 2.283386 | 1.36922 | 1.355528 |
| 1044 | 1.10787 | 1.096791 | 0.7987 | 0.790713 |
| 1056 | 0.54642 | 0.540956 | 0.68437 | 0.677526 |
| 1068 | 1.77409 | 1.756349 | 0.68029 | 0.673487 |
| 1080 | 1.82309 | 1.804859 | 0.95876 | 0.949172 |
| 1092 | 1.23097 | 1.21866 | 0.96041 | 0.950806 |
| 1104 | 1.38149 | 1.367675 | 0.97265 | 0.962924 |
| 1116 | 2.40694 | 2.382871 | 0.77421 | 0.766468 |
| 1128 | 2.24042 | 2.218016 | 0.49042 | 0.485516 |
| 1140 | 0.98477 | 0.974922 | 1.03627 | 1.025907 |
| 1152 | 1.21331 | 1.201177 | 0.61548 | 0.609325 |
| 1164 | 2.51465 | 2.489504 | 0.34927 | 0.345777 |
| 1176 | 1.30938 | 1.296286 | 0.23335 | 0.231017 |
| 1188 | 1.0386 | 1.028214 | 0.11668 | 0.115513 |
| 1200 | 1.52291 | 1.507681 | 0.07001 | 0.06931 |
| 1212 | 2.59986 | 2.573861 | 0.46129 | 0.456677 |
| 1224 | 1.43894 | 1.424551 | 1.06466 | 1.054013 |
| 1236 | 1.23186 | 1.219541 | 0.98436 | 0.974516 |
| 1248 | 0.68029 | 0.673487 | 0.64913 | 0.642639 |
| 1260 | 0.95876 | 0.949172 | 0.2549 | 0.252351 |
| 1272 | 1.9581 | 1.938519 | 0.54341 | 0.537976 |
| 1284 | 0.91186 | 0.902741 | 0.54818 | 0.542698 |
| 1296 | 1.16368 | 1.152043 | 0.31158 | 0.308464 |
| 1308 | 1.29824 | 1.285258 | 0.95876 | 0.949172 |
| 1320 | 2.68707 | 2.660199 | 0.69658 | 0.689614 |
| 1332 | 0.98477 | 0.974922 | 0.56649 | 0.560825 |
| 1344 | 0.39116 | 0.387248 | 0.5817 | 0.575883 |
| 1356 | 0.28002 | 0.27722 | 0.74323 | 0.735798 |
| 1368 | 0.11668 | 0.115513 | 0.95596 | 0.9464 |
| 1380 | 0.11668 | 0.115513 | 0.60085 | 0.594842 |
| 1392 | 0.51604 | 0.51088 | 0.56541 | 0.559756 |
| 1404 | 1.41937 | 1.405176 | 0.58773 | 0.581853 |
| 1416 | 1.8479 | 1.829421 | 0.86273 | 0.854103 |
| 1428 | 0.77895 | 0.771161 | 0.93562 | 0.926264 |
| 1440 | 0.2549 | 0.252351 | 0.71011 | 0.703009 |
| 1452 | 0.68029 | 0.673487 | 0.83091 | 0.822601 |
| 1464 | 0.80428 | 0.796237 | 1.31296 | 1.29983 |
| 1476 | 0.5193 | 0.514107 | 1.37747 | 1.363695 |
| 1488 | 0.95876 | 0.949172 | 0.58762 | 0.581744 |
| 1500 | 0.81817 | 0.809988 | 0.37496 | 0.37121 |
| 1512 | 0.90403 | 0.89499 | 0.77384 | 0.766102 |
| 1524 | 0.75059 | 0.743084 | 0.81806 | 0.809879 |
| 1536 | 0.83705 | 0.82868 | 0.70751 | 0.700435 |
| 1548 | 1.68298 | 1.66615 | 0.18029 | 0.178487 |
| 1560 | 1.39424 | 1.380298 | 1.25328 | 1.240747 |
| 1572 | 1.25646 | 1.243895 | 1.27301 | 1.26028 |
| 1584 | 1.18253 | 1.170705 | 0.63005 | 0.62375 |
| 1596 | 2.3993 | 2.375307 | 0.73324 | 0.725908 |
| 1608 | 1.04032 | 1.029917 | 0.99372 | 0.983783 |
| 1620 | 0.79888 | 0.790891 | 1.12946 | 1.118165 |
| 1632 | 1.04032 | 1.029917 | 0.77384 | 0.766102 |
| 1644 | 2.41257 | 2.388444 | 0.88194 | 0.873121 |
| 1656 | 2.28933 | 2.266437 | 1.27848 | 1.265695 |
| 1668 | 0.83946 | 0.831065 | 1.0909 | 1.079991 |
| 1680 | 0.44995 | 0.445451 | 0.9334 | 0.924066 |
| 1692 | 0.97282 | 0.963092 | 0.07147 | 0.070755 |
| 1704 | 0.81806 | 0.809879 | 1.1796 | 1.167804 |
| 1716 | 0.77384 | 0.766102 | 1.34812 | 1.334639 |
| 1728 | 0.23658 | 0.234214 | 0.66329 | 0.656657 |
| 1740 | 1.52291 | 1.507681 | 1.41937 | 1.405176 |
| 1752 | 1.85052 | 1.832015 | 1.30743 | 1.294356 |
| 1764 | 0.70005 | 0.69305 | 0.86273 | 0.854103 |
| 1776 | 0.73324 | 0.725908 | 0.64438 | 0.637936 |
| 1788 | 1.59673 | 1.580763 | 1.00643 | 0.996366 |
| 1800 | 1.70567 | 1.688613 | 1.22549 | 1.213235 |
| 1812 | 0.99493 | 0.984981 | 0.74814 | 0.740659 |
| 1824 | 0.93823 | 0.928848 | 0.70005 | 0.69305 |

TABLE X
VPD measurements with commercial (temperature sensor LM35,
Texas Instruments, TX and humidity sensor DHT11, Adafruit, NY)
and developed sensors when the plants were kept in shade
Table X. VPD of Plants kept in the Shade

| | VPD in | VPD in | VPD in | VPD in |
|---|---|---|---|---|
| | kPa with | kPa with | kPa with | kPa with |
| | our | commercial | our | commercial |
| | sensors | sensors | sensors | sensors |
| | (stressed | (stressed | (control | (control |
| | plant) | plant) | plant) | plant) |
| 12 | 0.70914 | 0.702049 | 0.77458 | 0.766834 |
| 24 | 1.11057 | 1.099464 | 0.64594 | 0.639481 |
| 36 | 0.86288 | 0.854251 | 0.70824 | 0.701158 |
| 48 | 0.61548 | 0.609325 | 0.46883 | 0.464142 |
| 60 | 0.7676 | 0.759924 | 0.80522 | 0.797168 |
| 72 | 1.25578 | 1.243222 | 0.45781 | 0.453232 |
| 84 | 1.06286 | 1.052231 | 0.57367 | 0.567933 |
| 96 | 0.91186 | 0.902741 | 0.49543 | 0.490476 |
| 108 | 1.01736 | 1.007186 | 0.76678 | 0.759112 |
| 120 | 1.38257 | 1.368744 | 0.62267 | 0.616443 |
| 132 | 0.86273 | 0.854103 | 0.51182 | 0.506702 |
| 144 | 0.75988 | 0.752281 | 0.70718 | 0.700108 |
| 156 | 1.46573 | 1.451073 | 0.962 | 0.95238 |
| 168 | 0.66619 | 0.659528 | 0.7987 | 0.790713 |
| 180 | 0.43272 | 0.428393 | 0.76669 | 0.759023 |
| 192 | 0.4881 | 0.483219 | 0.77895 | 0.771161 |
| 204 | 1.53262 | 1.517294 | 0.87367 | 0.864933 |
| 216 | 0.99576 | 0.985802 | 0.58689 | 0.581021 |
| 228 | 0.67347 | 0.666735 | 0.51421 | 0.509068 |
| 240 | 0.74146 | 0.734045 | 0.88308 | 0.874249 |
| 252 | 1.30793 | 1.294851 | 1.0105 | 1.000395 |
| 264 | 0.80428 | 0.796237 | 0.77539 | 0.767636 |
| 276 | 0.96434 | 0.954697 | 0.6054 | 0.599346 |
| 288 | 0.57123 | 0.565518 | 0.55274 | 0.547213 |
| 300 | 1.29854 | 1.285555 | 1.00643 | 0.996366 |
| 312 | 1.45744 | 1.442866 | 0.87367 | 0.864933 |
| 324 | 0.75737 | 0.749796 | 0.68029 | 0.673487 |
| 336 | 0.53532 | 0.529967 | 0.58338 | 0.577546 |
| 348 | 1.68456 | 1.667714 | 1.03169 | 1.021373 |
| 360 | 1.51893 | 1.503741 | 1.28357 | 1.270734 |
| 372 | 1.21331 | 1.201177 | 1.01109 | 1.000979 |
| 384 | 0.66329 | 0.656657 | 0.86168 | 0.853063 |
| 396 | 1.58344 | 1.567606 | 1.14727 | 1.135797 |
| 408 | 2.01173 | 1.991613 | 1.18064 | 1.168834 |
| 420 | 1.36465 | 1.351004 | 0.70267 | 0.695643 |
| 432 | 1.16675 | 1.155083 | 0.61548 | 0.609325 |
| 444 | 1.85518 | 1.836628 | 1.25685 | 1.244282 |
| 456 | 2.01861 | 1.998424 | 0.99576 | 0.985802 |
| 468 | 1.69142 | 1.674506 | 1.07791 | 1.067131 |
| 480 | 1.15711 | 1.145539 | 0.90878 | 0.899692 |
| 492 | 2.01328 | 1.993147 | 0.86218 | 0.853558 |
| 504 | 1.87786 | 1.859081 | 1.32068 | 1.307473 |
| 516 | 1.1244 | 1.113156 | 1.10013 | 1.089129 |
| 528 | 0.97944 | 0.969646 | 0.80781 | 0.799732 |
| 540 | 1.36903 | 1.35534 | 0.36395 | 0.360311 |
| 552 | 1.82005 | 1.80185 | 0.71793 | 0.710751 |
| 564 | 1.75197 | 1.73445 | 0.5193 | 0.514107 |
| 576 | 0.96071 | 0.951103 | 0.6401 | 0.633699 |
| 588 | 0.42942 | 0.425126 | 1.24202 | 1.2296 |
| 600 | 2.16742 | 2.145746 | 0.78737 | 0.779496 |
| 612 | 1.24202 | 1.2296 | 0.63618 | 0.629818 |
| 624 | 0.68908 | 0.682189 | 0.20415 | 0.202109 |
| 636 | 0.36395 | 0.360311 | 0.13279 | 0.131462 |
| 648 | 0.71793 | 0.710751 | 0.64913 | 0.642639 |
| 660 | 0.5193 | 0.514107 | 0.21002 | 0.20792 |
| 672 | 0.77468 | 0.766933 | 0.69631 | 0.689347 |
| 684 | 1.06466 | 1.054013 | 1.20513 | 1.193079 |
| 696 | 1.51985 | 1.504652 | 0.95876 | 0.949172 |
| 708 | 1.68113 | 1.664319 | 0.69419 | 0.687248 |
| 720 | 0.22749 | 0.225215 | 1.26136 | 1.248746 |
| 732 | 0.54341 | 0.537976 | 1.14727 | 1.135797 |
| 744 | 0.75299 | 0.74546 | 0.81716 | 0.808988 |
| 756 | 0.28002 | 0.27722 | 0.87599 | 0.86723 |
| 768 | 0.36016 | 0.356558 | 1.18062 | 1.168814 |
| 780 | 1.74086 | 1.723451 | 1.26136 | 1.248746 |
| 792 | 1.18285 | 1.171022 | 0.75737 | 0.749796 |
| 804 | 0.9334 | 0.924066 | 0.55274 | 0.547213 |
| 816 | 1.98133 | 1.961517 | 0.81864 | 0.810454 |
| 828 | 1.37517 | 1.361418 | 1.30192 | 1.288901 |
| 840 | 0.81716 | 0.808988 | 1.00977 | 0.999672 |
| 852 | 0.82124 | 0.813028 | 0.81673 | 0.808563 |
| 864 | 2.04416 | 2.023718 | 0.57942 | 0.573626 |
| 876 | 2.25825 | 2.235668 | 1.18064 | 1.168834 |
| 888 | 1.66924 | 1.652548 | 0.95825 | 0.948668 |
| 900 | 0.88439 | 0.875546 | 0.64913 | 0.642639 |
| 912 | 2.42741 | 2.403136 | 1.03627 | 1.025907 |
| 924 | 1.94082 | 1.921412 | 1.51144 | 1.496326 |
| 936 | 1.44253 | 1.428105 | 0.99372 | 0.983783 |
| 948 | 0.70005 | 0.69305 | 0.82124 | 0.813028 |
| 960 | 1.4662 | 1.451538 | 0.54261 | 0.537184 |
| 972 | 2.51699 | 2.49182 | 1.18371 | 1.171873 |
| 984 | 1.95144 | 1.931926 | 0.46129 | 0.456677 |
| 996 | 1.42808 | 1.413799 | 0.6401 | 0.633699 |
| 1008 | 2.27437 | 2.251626 | 1.30192 | 1.288901 |
| 1020 | 2.06527 | 2.044617 | 0.84759 | 0.839114 |
| 1032 | 2.1288 | 2.107512 | 0.63864 | 0.632254 |
| 1044 | 1.36873 | 1.355043 | 0.49274 | 0.487813 |
| 1056 | 0.95876 | 0.949172 | 0.86218 | 0.853558 |
| 1068 | 2.43329 | 2.408957 | 0.54341 | 0.537976 |
| 1080 | 0.81716 | 0.808988 | 0.86273 | 0.854103 |
| 1092 | 0.86168 | 0.853063 | 0.72126 | 0.714047 |
| 1104 | 2.51582 | 2.490662 | 1.18062 | 1.168814 |
| 1116 | 2.45937 | 2.434776 | 1.24262 | 1.230194 |
| 1128 | 0.86273 | 0.854103 | 0.90935 | 0.900257 |
| 1140 | 0.95811 | 0.948529 | 0.81673 | 0.808563 |
| 1152 | 0.86218 | 0.853558 | 0.4121 | 0.407979 |
| 1164 | 0.62554 | 0.619285 | 0.23335 | 0.231017 |
| 1176 | 0.95876 | 0.949172 | 0.11668 | 0.115513 |
| 1188 | 0.86552 | 0.856865 | 0.11668 | 0.115513 |
| 1200 | 2.16191 | 2.140291 | 0.54341 | 0.537976 |
| 1212 | 2.56326 | 2.537627 | 1.12133 | 1.110117 |
| 1224 | 1.66924 | 1.652548 | 1.1356 | 1.124244 |
| 1236 | 0.9334 | 0.924066 | 0.98549 | 0.975635 |
| 1248 | 0.5168 | 0.511632 | 0.11776 | 0.116582 |
| 1260 | 0.28002 | 0.27722 | 0.48705 | 0.48218 |
| 1272 | 0.11668 | 0.115513 | 0.46129 | 0.456677 |
| 1284 | 0.11668 | 0.115513 | 0.36929 | 0.365597 |
| 1296 | 0.54341 | 0.537976 | 1.32516 | 1.311908 |
| 1308 | 1.49453 | 1.479585 | 0.95876 | 0.949172 |
| 1320 | 1.3839 | 1.370061 | 0.61015 | 0.604049 |
| 1332 | 0.95811 | 0.948529 | 0.75341 | 0.745876 |
| 1344 | 0.167 | 0.16533 | 0.57751 | 0.571735 |
| 1356 | 0.48705 | 0.48218 | 0.84885 | 0.840362 |
| 1368 | 0.59816 | 0.592178 | 0.74086 | 0.733451 |
| 1380 | 0.44315 | 0.438719 | 0.83764 | 0.829264 |
| 1392 | 1.94421 | 1.924768 | 1.31296 | 1.29983 |
| 1404 | 0.95876 | 0.949172 | 1.22584 | 1.213582 |
| 1416 | 0.70862 | 0.701534 | 0.66339 | 0.656756 |
| 1428 | 0.99133 | 0.981417 | 0.56294 | 0.557311 |
| 1440 | 0.62418 | 0.617938 | 0.64438 | 0.637936 |
| 1452 | 2.09586 | 2.074901 | 0.74774 | 0.740263 |
| 1464 | 1.39424 | 1.380298 | 0.73911 | 0.731719 |
| 1476 | 1.29834 | 1.285357 | 0.70751 | 0.700435 |
| 1488 | 2.3771 | 2.353329 | 0.37496 | 0.37121 |
| 1500 | 2.66214 | 2.635519 | 0.43499 | 0.43064 |
| 1512 | 1.14503 | 1.13358 | 0.48009 | 0.475289 |
| 1524 | 1.03206 | 1.021739 | 0.77384 | 0.766102 |
| 1536 | 1.55316 | 1.537628 | 0.95876 | 0.949172 |
| 1548 | 2.09586 | 2.074901 | 1.11881 | 1.107622 |
| 1560 | 1.88885 | 1.869962 | 0.54341 | 0.537976 |
| 1572 | 1.23814 | 1.225759 | 0.60085 | 0.594842 |
| 1584 | 0.44995 | 0.445451 | 1.19597 | 1.18401 |
| 1596 | 0.83297 | 0.82464 | 1.39131 | 1.377397 |
| 1608 | 0.63486 | 0.628511 | 0.6054 | 0.599346 |
| 1620 | 0.88439 | 0.875546 | 0.59064 | 0.584734 |
| 1632 | 1.43894 | 1.424551 | 0.62151 | 0.615295 |
| 1644 | 2.48072 | 2.455913 | 1.23843 | 1.226046 |
| 1656 | 0.95403 | 0.94449 | 0.68029 | 0.673487 |
| 1668 | 0.53084 | 0.525532 | 0.58338 | 0.577546 |
| 1680 | 1.86773 | 1.849053 | 0.14845 | 0.146966 |
| 1692 | 2.77942 | 2.751626 | 1.18062 | 1.168814 |
| 1704 | 1.36528 | 1.351627 | 1.03627 | 1.025907 |
| 1716 | 0.76752 | 0.759845 | 0.58338 | 0.577546 |
| 1728 | 1.04032 | 1.029917 | 0.95876 | 0.949172 |
| 1740 | 2.74255 | 2.715125 | 0.48705 | 0.48218 |
| 1752 | 1.0909 | 1.079991 | 0.74086 | 0.733451 |
| 1764 | 0.9334 | 0.924066 | 1.47115 | 1.456439 |
| 1776 | 0.10423 | 0.103188 | 0.86218 | 0.853558 |
| 1788 | 2.04416 | 2.023718 | 0.74814 | 0.740659 |
| 1800 | 2.2331 | 2.210769 | 0.70005 | 0.69305 |
| 1812 | 0.9334 | 0.924066 | 0.57751 | 0.571735 |
| 1824 | 1.27888 | 1.266091 | 0.73324 | 0.725908 |

### Example 16

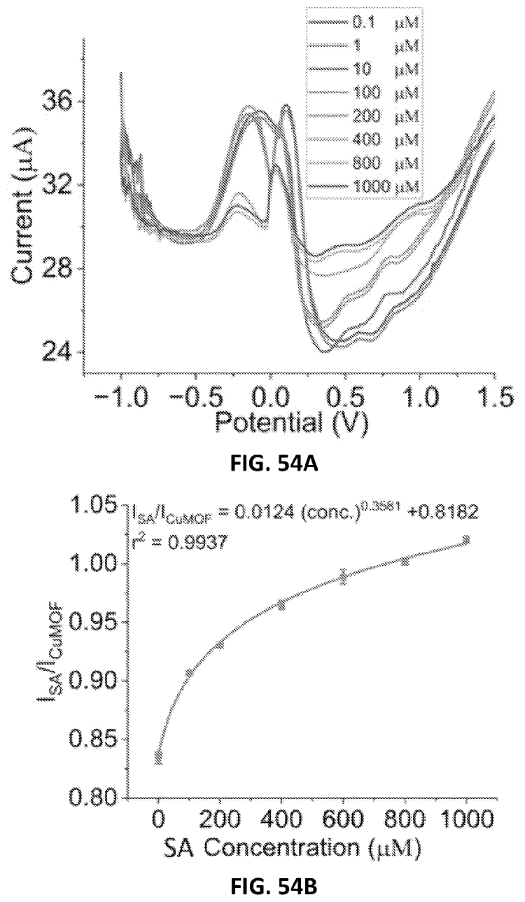

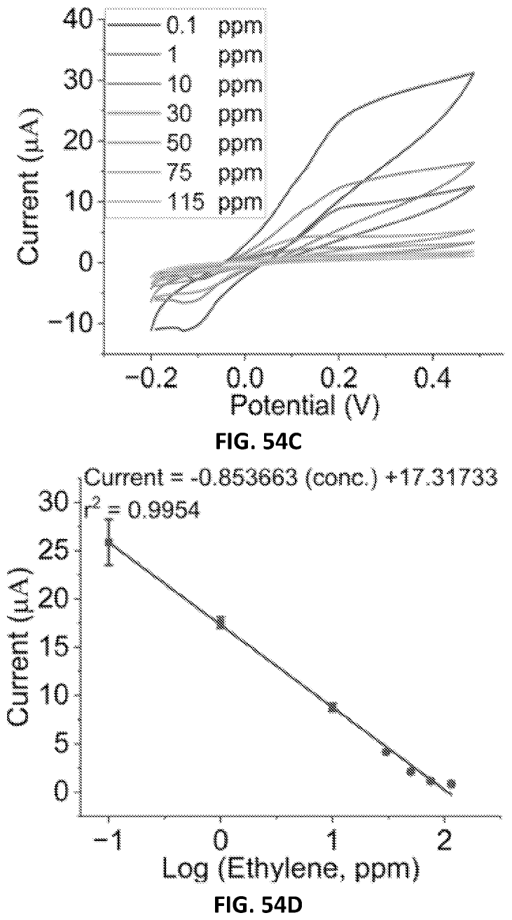

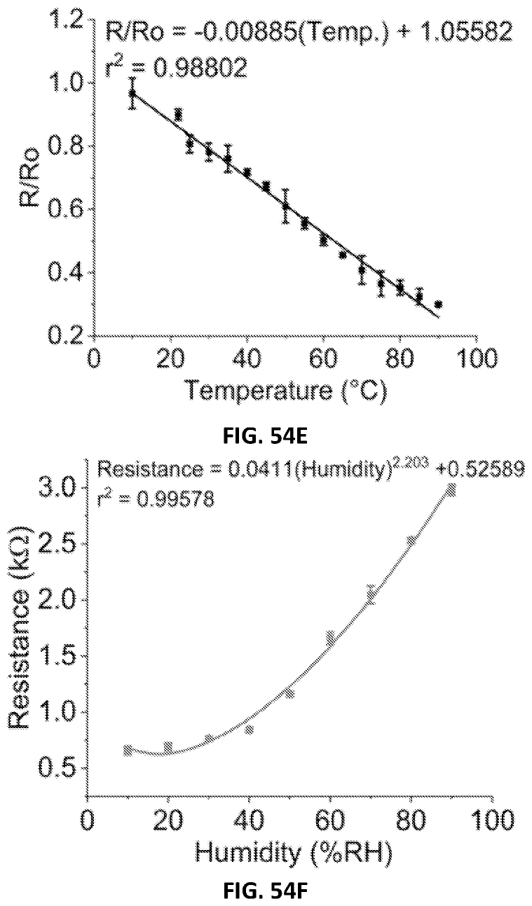

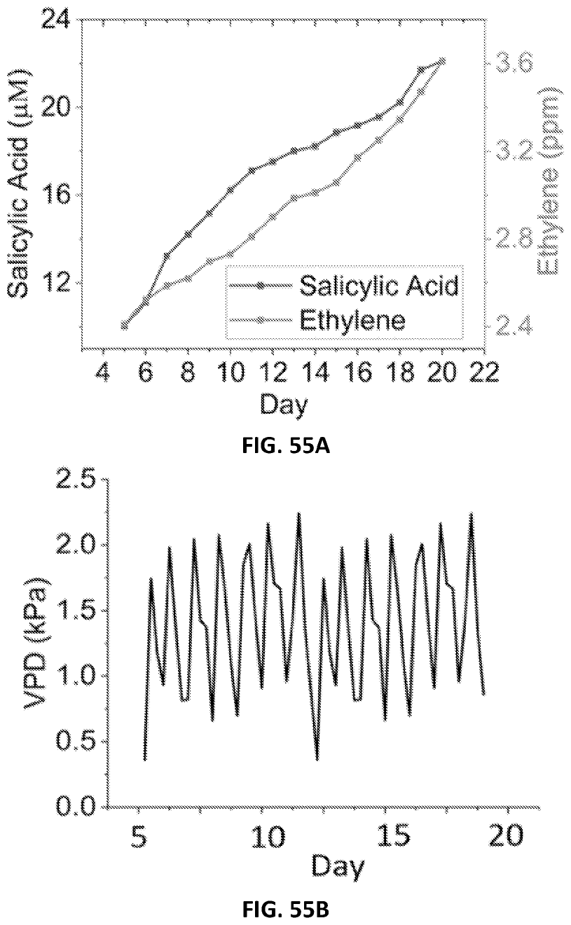

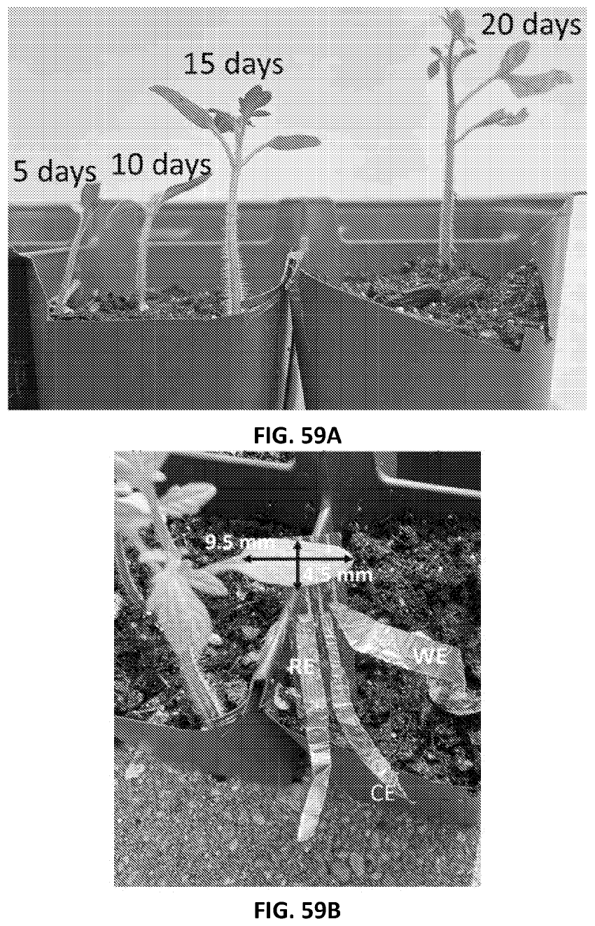

Water Stress Experiments at Different Growth Stages of Tomato Plants. SA, ethylene, and VPD levels were also measured at different growth stages of a plant. Tomato seedlings were grown to conduct this investigation owing to their conducive growth during the study period (from March to April). SA, ethylene, and VPD levels were measured in plants aged 5, 10, 15, and 20 days, counted from germination. FIG. 59A depicts the optical images of plants on different days of growing. The sensor patch was reconfigured to fit into the smallest leaf. The same sensor patch was used to measure SA, ethylene, and VPD levels at 5, 10, 15, and 20 days old plants. FIG. 59B depicts the sensor patch installed on a 15-day-old tomato plant. The calibration graphs of the modified sensors are shown in FIGS. 54A-54F and real-time SA, ethylene, and VPD measurements are shown in FIGS. 55A-55B.

### Example 17

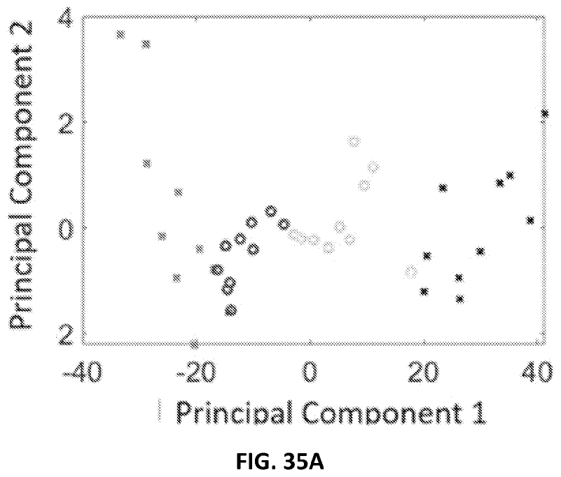

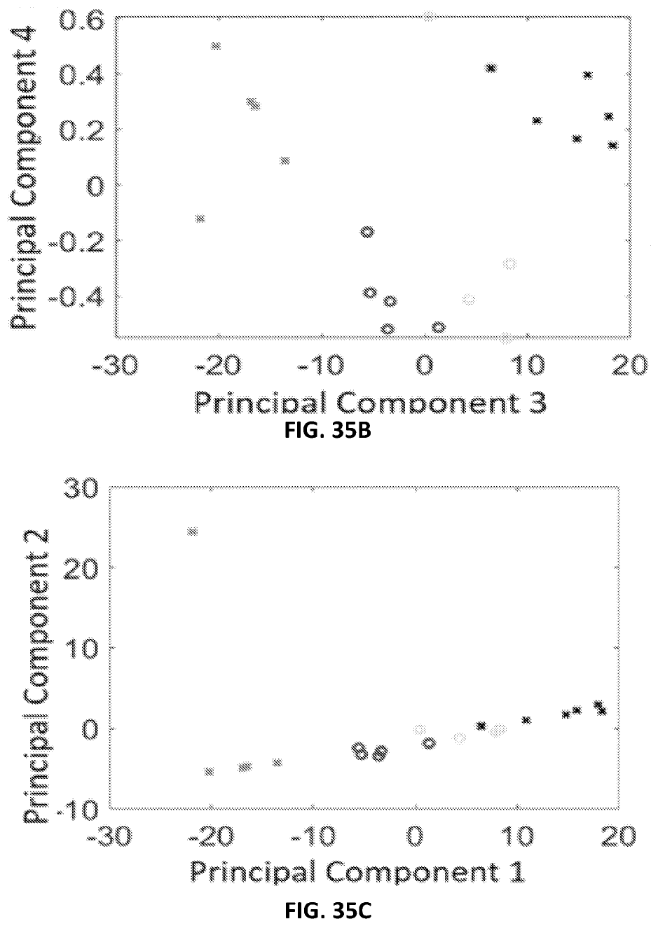

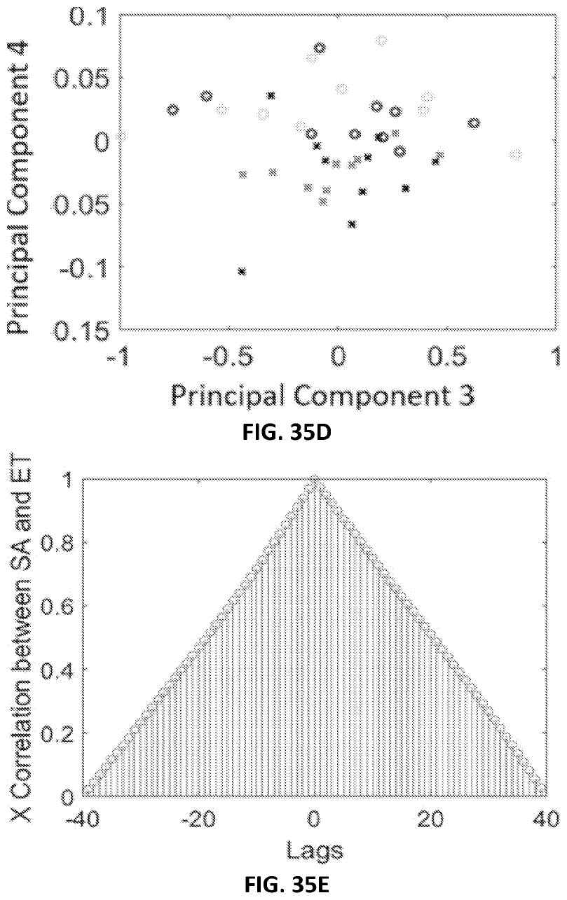

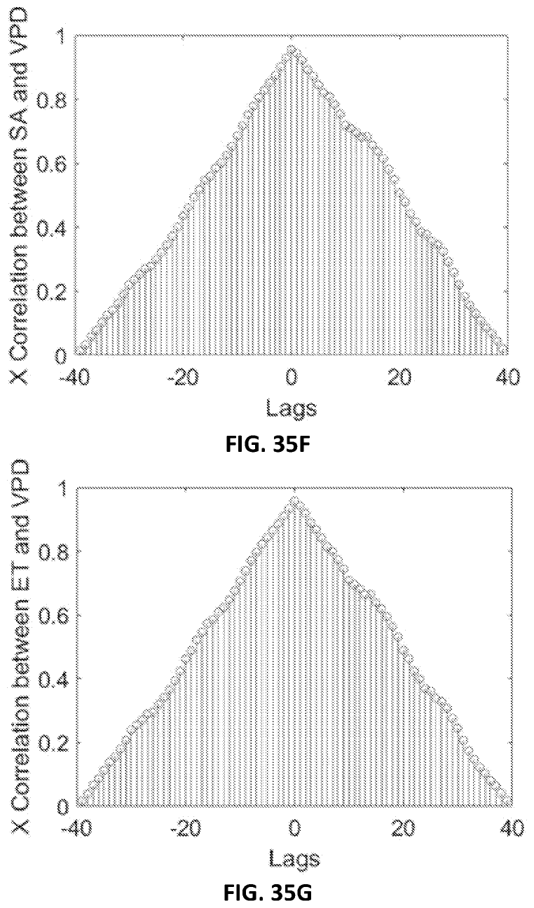

Feature Analysis. The sensor measurements were analyzed by a principal component analysis (PCA)-based pattern recognition algorithm. The results demonstrate SA and ethylene levels can distinguish chunks of stress levels in plants. The experiment was conducted over 40 days and every 10 days in a row was considered one stress period. Ten plants were used for this experiment. The four stress periods were defined by differing amounts of water applied to the plants. SA and ethylene levels, and the number of days were the input variables to the PCA algorithm. The plot of principal component 1 versus principal component 2 showed a clear distinction between the four stress periods (FIG. 35A). However, as expected, the principal components 3 and 4 could not provide clear identifiable separation among the four stress periods (FIG. 35B). The red cross, blue circle, green circle, and black cross symbols represent 0-10 days, 11-20 days, 21-30 days, and 31-40 days of water stress, respectively. Here, ET=ethylene, VPD=vapor pressure deficit, and ‘X’ is used as the short form for ‘cross’.

A similar analysis was conducted at four different growth stages of tomato plants. A total of 16 plants were used for this study with 4 plants per growth stage. A noticeable separation among the growth stages was observed in the principal component 1 versus principal component 2 plot. The red cross symbols in FIGS. 35C-35D indicate 0-5 days of growth, blue circles represent 6-10 days of growth, green circles represent 11-15 days of growth, and black crosses represent 16-20 days of growth.

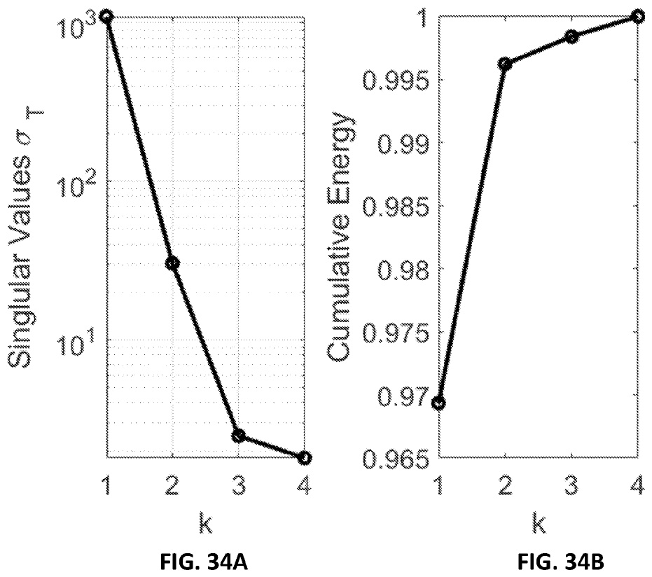

Further investigation was carried out wherein the singular value deposition and corresponding cumulative energy were analyzed (FIGS. 34A-34B), where, k represents the principal component number. The first two principal components have larger singular values. In addition, the cumulative energy analysis demonstrates that the first principal component captures almost 97% of the total energy. These results confirm that the most significant features can be reliably extracted by the first two principal components. Principal components 1 and 2 were found to possess higher cumulative energy, thereby justifying their use in differentiating the stress and growth periods in plants. These results suggest that the hormone levels (SA and ethylene) could clearly distinguish different growth and stress stages in plants.

A cross-correlation analysis was performed to identify the association between the measured crop parameters (SA, ethylene, and VPD). For this study, 10 bell pepper plants were subjected to water stress. Leaf SA and ethylene levels were measured once a day (1:30 P.M.), while temperature and humidity values were recorded four times a day (8.00 a.m., 12.00 p.m., 4.00 p.m., and 8.00 p.m.), over 40 days. The normalized cross-correlation coefficients are plotted in FIGS. 35E-35G. A symmetrical triangular shape with respect to lag=0 signifies a high similarity between the two parameters under consideration. It is evident from FIGS. 35E-35G that the following pairs are highly similar: SA and ethylene; SA and VPD; ethylene and VPD, as is also suggested by the dynamic plots in FIGS. 41A-41F. The beginning of lag at −40 and ending at +40 represents 40 days of data collection. It is noteworthy that the cross-correlation between SA and ethylene had a perfect triangular shape. In contrast, due to the oscillatory nature of VPD, the normalized cross-correlation coefficients between SA and VPD as well as ethylene and VPD showed a slightly distorted triangular shape. These results conclude the significant correlation between SA, ethylene, and VPD levels in water-stressed plants.

### Example 18

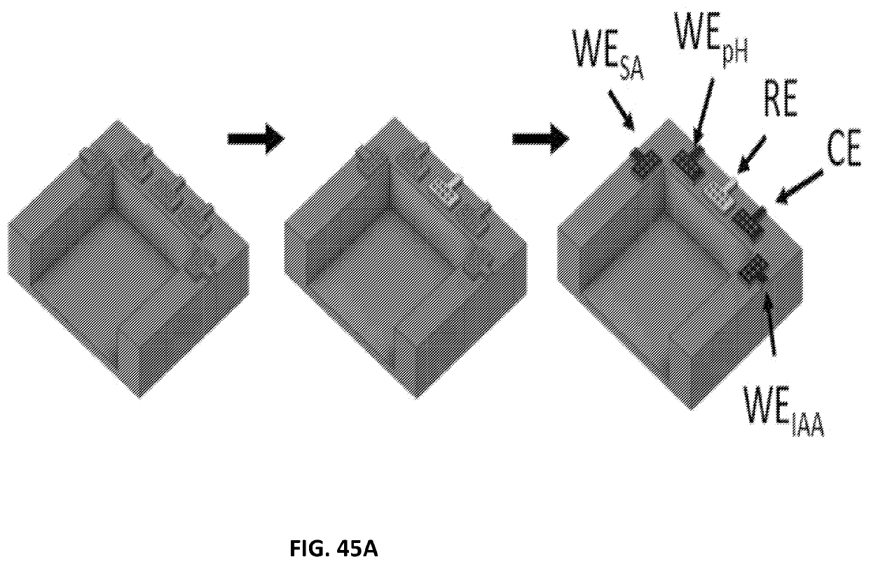

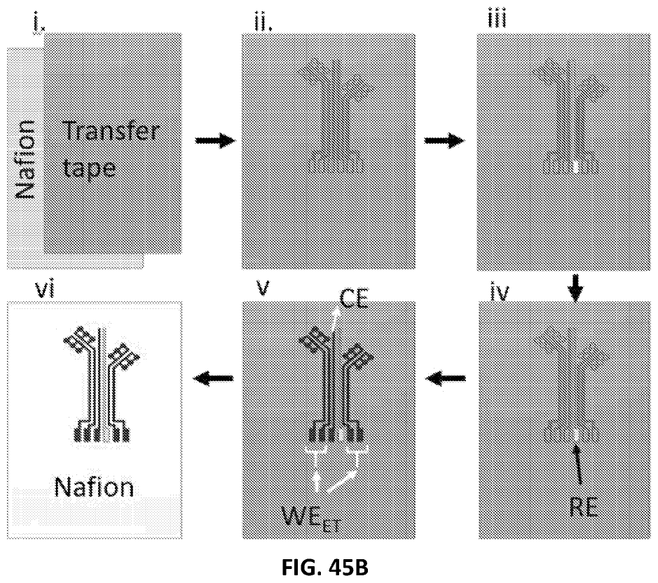

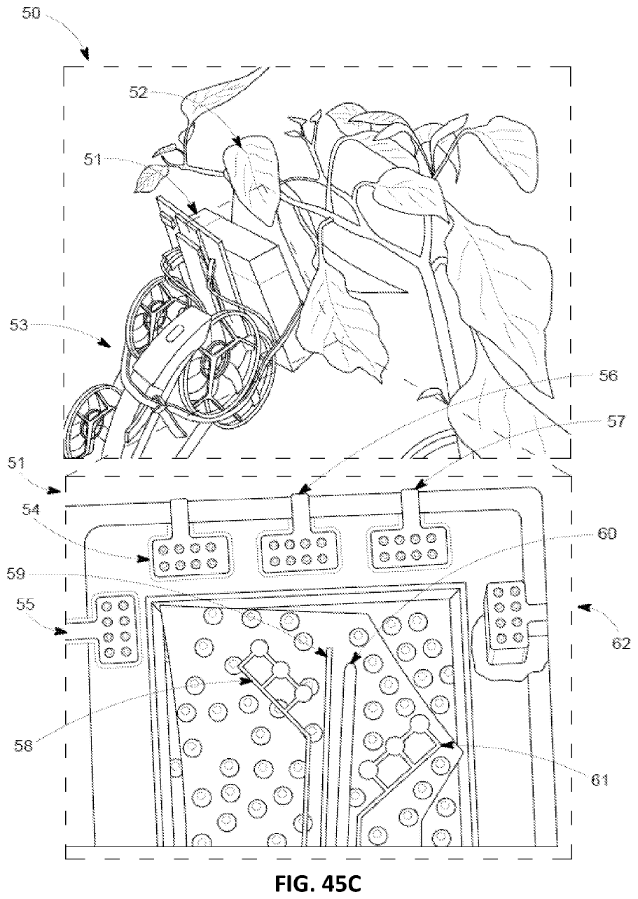

Electrode fabrication for a sensor suite. A sensor suite comprising five microneedle arrays was fabricated as follows. One array worked as the shared reference electrode (RE), one as the shared counter electrode (CE), and the other three arrays served as working electrodes for SA, IAA and pH sensors (WE_{SA}, WE_{IAA}, and WE_{pH}, respectively). To prepare the sensor suite, a 4 cm×3 cm×0.8 cm box was designed with an open ceiling and three sidewalls, as depicted in FIG. 45A. The ethylene sensor was placed inside a chamber enclosed by the three sidewalls, whereas the microneedle sensors were laid on top of the sidewalls. Each microneedle array consisted of eight pyramid-shaped microneedles, each having a square base of 800 μm, a height of 800 μm, and a tip angle of 60°. A Form 3B stereolithography printer was used to print the 3D box with the microneedles. BioMed Clear resin was used as the printing material to ensure biocompatibility of the microneedles.

The ethylene sensor was fabricated by a screen printing process, as shown in FIG. 45B. A thin Nafion sheet was used as the substrate material because it works as a solid-state electrolyte. To prepare the ethylene sensor, Nafion was covered with a transfer tape that worked as the stencil mask (i). Electrode patterns cut by a benchtop cutter (ii) and transfer tape from the reference electrode region was removed (iii). The reference electrode was printed with Ag/AgCl paste (iv). The working and counter electrode areas were exposed and printed with graphene ink (v), resulting in a dual working electrode for ethylene (WE_{ET}). As a result, the ethylene sensitivity was increased to sub-ppm levels. The electrodes were cured at 80° C. for 60 minutes. The transfer tape was then removed resulting in electrodes transferred to the Nafion sheet (vi).

Synthesis of SA and IAA Selective Coatings. The working electrode of the SA sensor (WE_{SA}) was functionalized by a copper metal-organic framework (CuMOF)/nafion/carbon black nanocomposite, as described above. 4 μL of the nanocomposite solution was drop cast on WE_{SA} and dried at room temperature.

The IAA working electrode (WE_{1AA}) was modified with gold nanoparticles decorated graphene hydrogel nanocomposite. Briefly, 1 mg mL⁻¹ of graphene oxide suspension was prepared. 20 mL of this suspension was added to 2 mL of 0.8511 mg mL⁻¹ chloroauric acid solution and 1 mL of triethylenetetramine. The resulting mixture was sonicated for 10 minutes and then heated at 140° C. for 12 h. The obtained AuNP-GHs were cooled down to room temperature and then freeze-dried for 24 h to form powders that were stored in a desiccator. 4 μL of the as-prepared composite solution was drop cast on WE_{IAA} to form an electrode.

Synthesis of Ethylene Selective Coating. WE_{ET} was functionalized with a composite copper complex (I)-single-walled carbon nanotube coating for selective measurement of ethylene gas. All operations were carried out under an atmosphere of purified nitrogen and all solutions were prepared in deionized water. To prepare Na [HB(3,5-(CF₃)₂-pz)₃]), 0.40 g (10.6 mmol) of NaBH₄ and 7.55 g (37 mmol) of 3,5-(CF₃)₂-pz were mixed in just as much kerosene as was needed to form a homogeneous mixture. The mixture was slowly heated to 180-190° C. and kept for 4 hr at 190° C. The flux was partially submerged in silicone oil during the heating process. The solution was occasionally (every 15 minutes) heated with a heat gun until pyrazole melted. During this period, a white solid slowly precipitated. After the mixture was cooled to room temperature, the resulting white solid was collected by suction filtration in air. It was washed several times with petroleum benzene and sucked dry in air to obtain Na [HB(3,5-(CF₃)₂-pz)₃]) as a white solid. Next, in order to form the Cu complex-1 coating, 8 mg of [CF₃SO₃Cu]₂·C₆H₆ were dissolved in 3 mL dry, degassed toluene. Finally, 17 mg of the freshly prepared Na[HB(3,5-(CF₃)₂-pz)₃]) were added to the mixture and stirred for 20 hrs at room temperature. The reaction mixture was filtrated through a Whatman 0.02 μm syringe filter and a colorless solution of Cu complex-1 with a concentration of ˜6 μmol/mL (6 mM) was obtained. The prepared solution was stored at 4° C. in a refrigerator until further use. In a separate tube, 0.5 mg of single-walled carbon nanotube (SWCNT) was added to a mixture of 0.8 mL 1,2-dichlorobenzene and 1.16 mL toluene, and the resulting mixture was sonicated for 2 hours to prepare a homogeneous solution. Next, the freshly prepared copper complex-1 solution was added to this mixture and sonicated for another 1 hour. Finally, 30 μL of this solution was drop cast on the working electrode (WE_{ET}) of the ethylene sensor.

Synthesis of pH Selective Coating. WE_{pH} was modified with polyaniline (PANI) nanofibers via electrodeposition. Polyaniline (PANI) nanofibers were deposited onto the graphene WE, as PANI-based electrode is highly sensitive to H₃O+ ions. In addition, the redox equilibrium between the H₃O+ and PANI provides high surface area, potential stability, biocompatibility, and reproducible performance. The PANI coating was deposited on the WE via electropolymerization method. The pH sensor electrodes were immersed in a mixture of 0.1 M aniline and 0.1 M HCl followed by 85 cycles of cyclic voltammetry (CV) for −0.2V to 0.6V at a 50 mV/s scan rate.

### Example 19

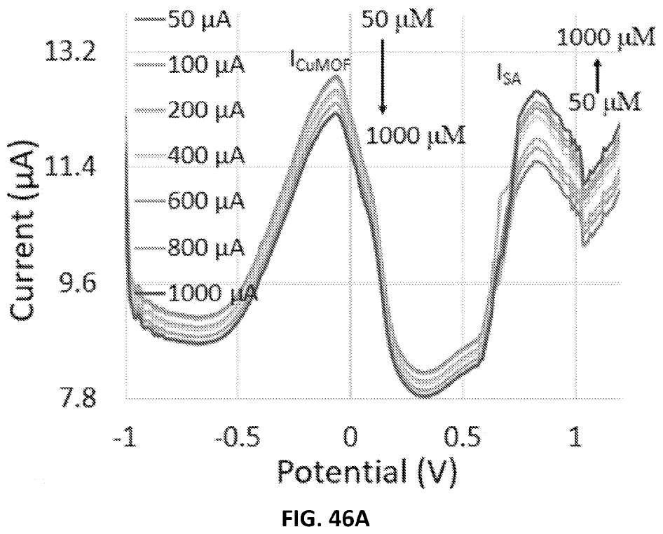

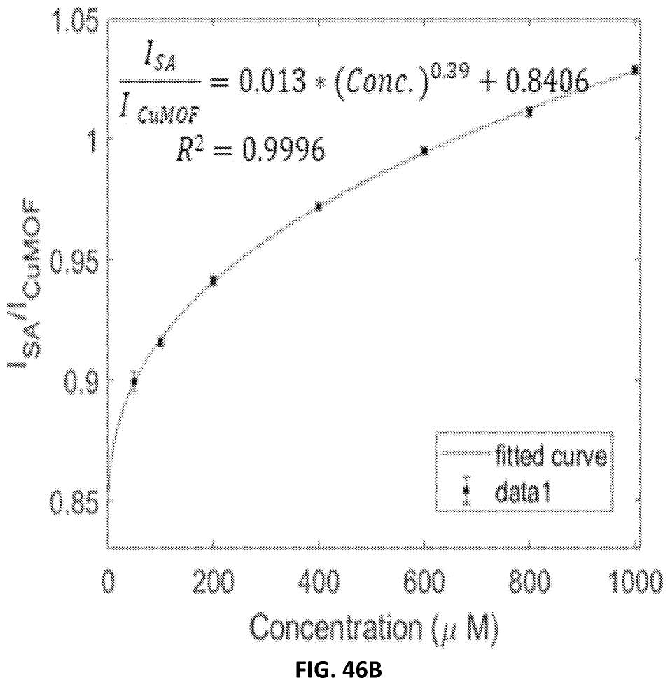

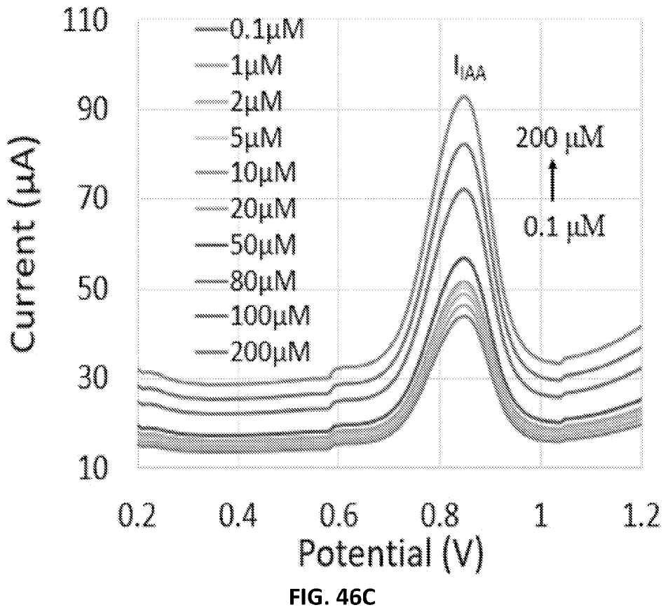

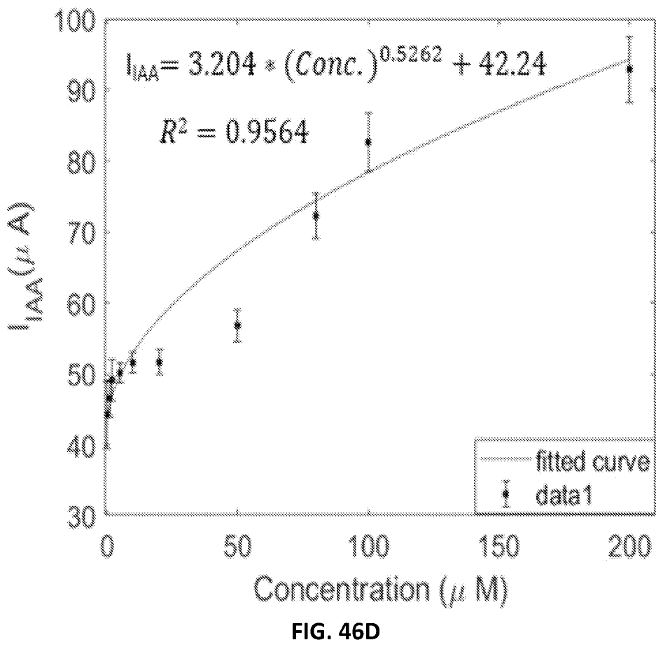

Electrochemical Detection of SA, IAA, and Ethylene. Electrochemical measurements were performed using differential pulse voltammetry (DPV) in a potential range from −1.0V to 1.2V for SA and from 0.2V to 1.2V for IAA (FIG. 46A and FIG. 46C). The SA sensor was calibrated for SA levels ranging from 50 M to 1000 M (FIG. 46B), while the IAA sensor was calibrated for IAA levels varying from 0.1 M to 200 M (FIG. 46D), commensurate with the typical SA and IAA concentrations found in plants. A ratiometric approach was used to calibrate the SA sensor, wherein the ratio of SA and CuMOF redox current peaks (I_{SA}/I_{CuMOF}) was plotted as a function of SA concentration and a power series curve was fitted to the data points (FIG. 46B). The SA and IAA sensors exhibited sensitivities of 0.005 μM⁻¹ and 0.8325 μA μM⁻¹, with detection down to 0.93 M and 0.08 M, respectively.

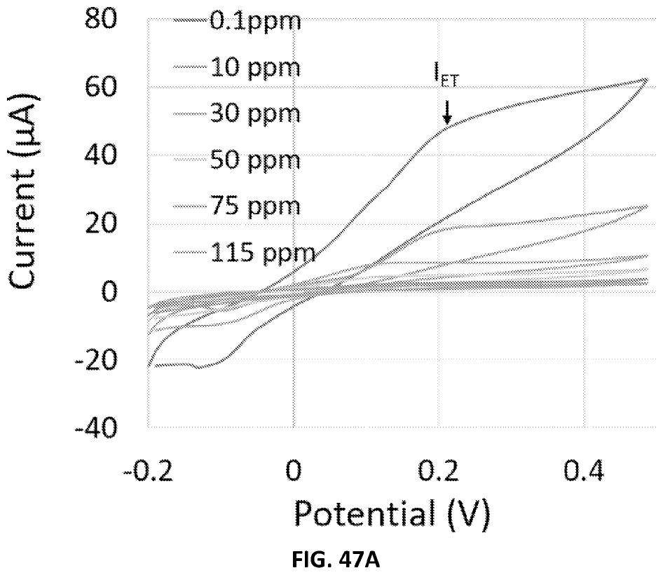

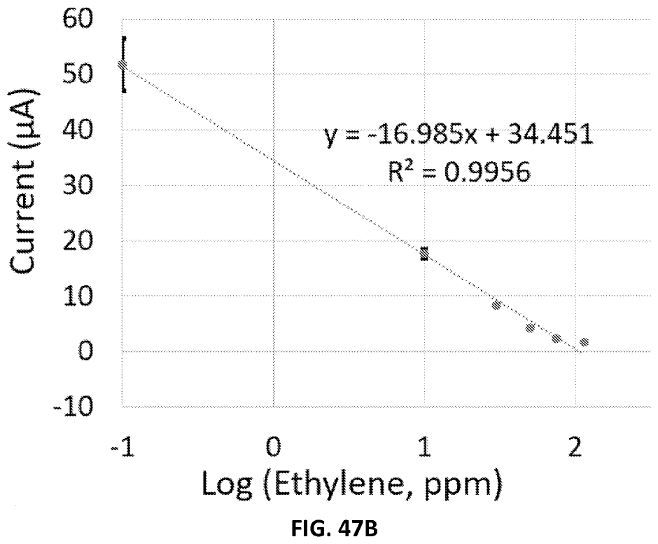

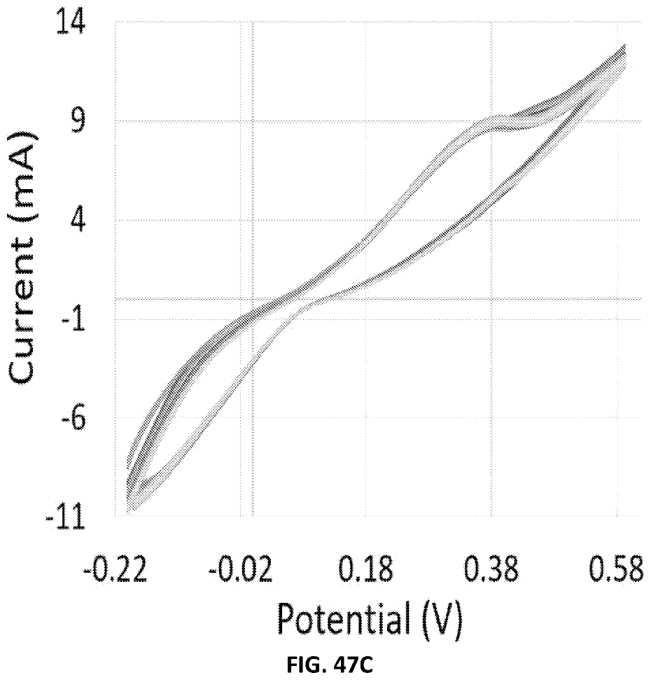

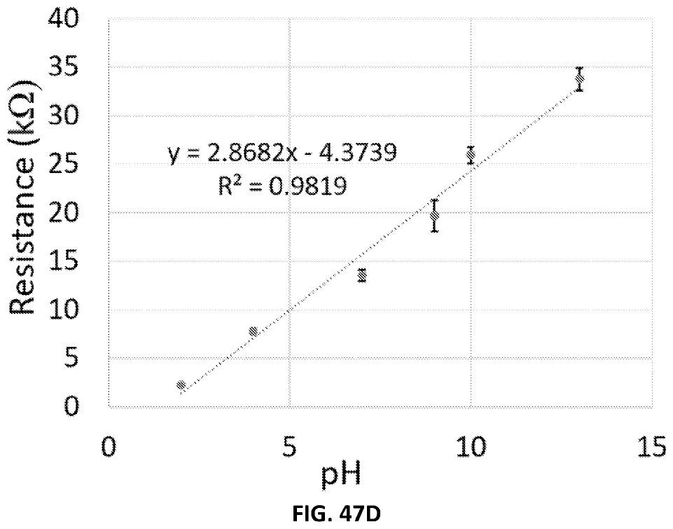

Cyclic Voltammetry (CV) method was used to conduct electrochemical characterization of the ethylene sensor. Different concentrations of ethylene gas were generated by controlling the gas flow rate and time in a flow chamber. The concentrations ranging from 0.1 ppm to 115 ppm were used to calibrate the ethylene sensor (FIG. 47A). The CV responses depict that the ethylene oxidation peak current (I_{ET}) lies between 0.12V and 0.17V. Upon exposure to a higher concentration of ethylene, the oxidation peak current decreased because ethylene molecules blocked the active sites in the carbon nanotube coating (FIG. 47A-47B).

pH Sensor Characterization. The pH sensor was calibrated with plant sap. The sap pH was varied by adding 0.1 M HCl and 0.01 M NaOH. CV responses for PANI deposition are shown in FIG. 47C. The pH sensor demonstrated an increase in the resistance measured across the electrodes with increasing pH value, as is illustrated in FIG. 47D.

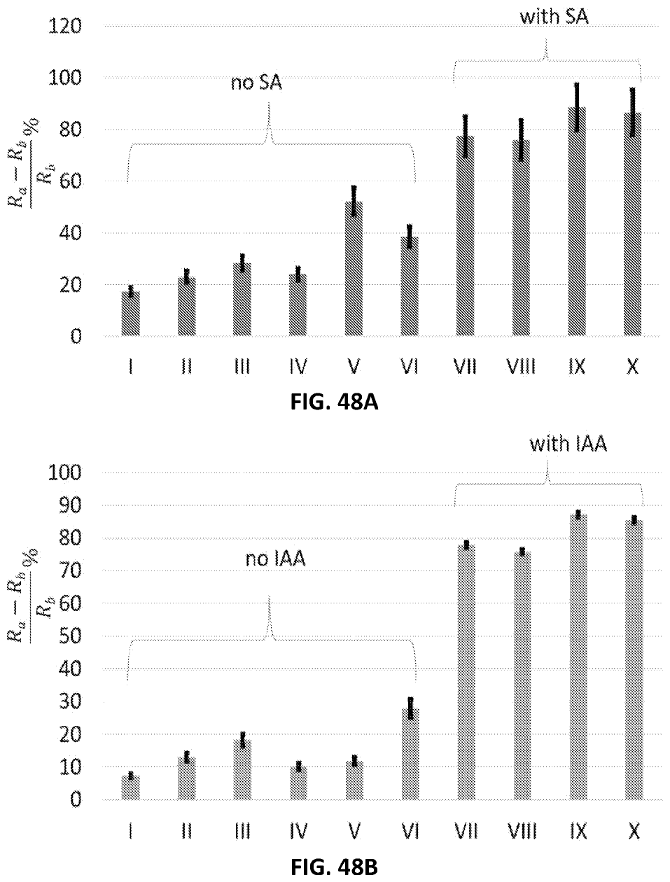

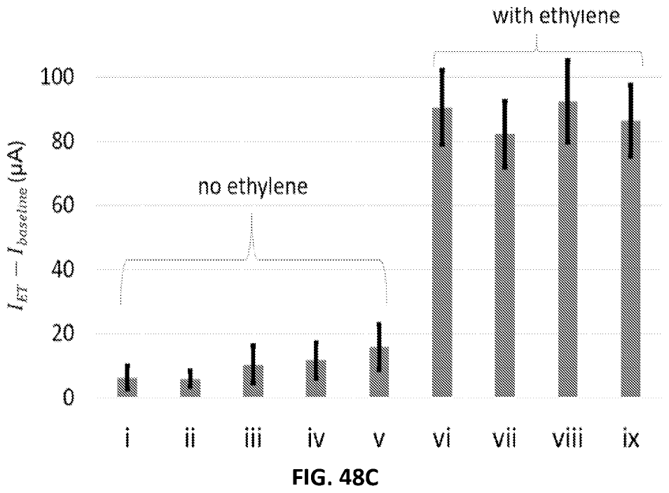

Selectivity Test for SA and IAA. The SA and IAA sensors were tested against several interfering species typically found in fruits/vegetables. Specifically, both SA and IAA sensors were tested under the following conditions shown in FIGS. 48A-48B: (i) Jasmonic acid (JA)=50 μM, (ii) L-Cysteine (L-Cys)=50 μM, (iii) glucose=50 μM, (iv) citric acid=50 μM, (v) ascorbic acid=50 μM, (vi) a mixture of JA, L-Cys, glucose, citric acid, and ascorbic acid (50 μM each), (vii) target hormone (SA/IAA)=100 μM, (viii) a mixture of ascorbic acid, JA, L-Cys, glucose, citric acid, ascorbic acid (50 μM each), and target hormone=100 μM, (ix) target (SA=900 μM or IAA=200 μM), (x) a mixture of ascorbic acid, JA, L-Cys, glucose, citric acid, ascorbic acid (50 μM each), and target (SA=900 μM or IAA=200 μM). In the absence of either SA or IAA (conditions (i) to (vi)), negligible responses were observed. However, when SA or IAA were present, respectively (conditions (vii) to (x)), a significant response was detected from each sensor. Rₐ=I_{SA}/I_{CuMOF} for SA sensor and I_{IAA} for IAA sensor, R_{b}=I_{baseline}/I_{CuMOF} for SA sensor and I_{baseline} for IAA sensor.

Selectivity Test for Ethylene. The ethylene sensor was tested against some common interfering gases emitted in an agricultural field. Specifically, ethylene sensor was tested under the following conditions shown in FIG. 48C: (i) 50 ppm N₂, (ii) 50 ppm CH₄, (iii) 50 ppm N₂O, (iv) 50 ppm NH₃, (v) a mixture of 50 ppm of N₂, CH₄, N₂O, NH₃ each, (vi) 10 ppm ethylene, (vii) a mixture of 50 ppm N₂, CH₄, N₂O, NH₃ each and 10 ppm ethylene, (viii) 115 ppm ethylene and (ix) a mixture of 50 ppm N₂, CH₄, N₂O, NH₃ each and 115 ppm ethylene. In the absence of ethylene (conditions (i) to (v)), negligible responses were observed. However, when ethylene was present (conditions (vi) to (ix)), a significant response was detected.

Thus, as shown in FIGS. 48A-48C, all three sensors exhibited negligible responses in the absence of the target analyte, thereby confirming excellent selectivity.

pH Correction of SA and IAA. The SA and IAA values measured with the above described sensors were corrected for pH variations in bell pepper at different stages of ripening. The pH correction was performed using the equations below. The pH value of 7 was considered as a reference.

$${f}_{intercept ( pH )} = \frac{intercept ( pH )}{intercept ( pH = 7 )} \qquad ( 23 )$$

$${f}_{s l o p e ( p H )} = \frac{slope ( pH )}{slope ( pH = 7 )} \qquad ( 24 )$$

$${f}_{exponent ( p H )} = \frac{exponent ( pH )}{exponent ( pH = 7 )} \qquad ( 25 )$$

The corrected intercept, slope, and exponent were found using the values computed in Equations (23-25):

$$intercept ( corrected ) = {f}_{intercept} ( pH ) * intercept ( initial ) \qquad ( 26 )$$

$$slope ( corrected ) = {f}_{slope} ( pH ) * slope ( initial ) \qquad ( 27 )$$

$$exponent ( corrected ) = {f}_{exponent} ( pH ) * exponent ( initial ) \qquad ( 28 )$$

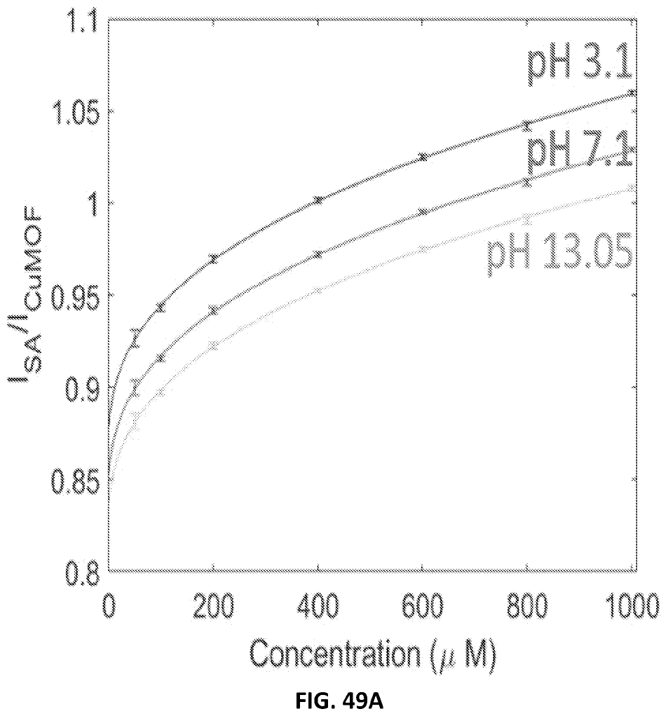

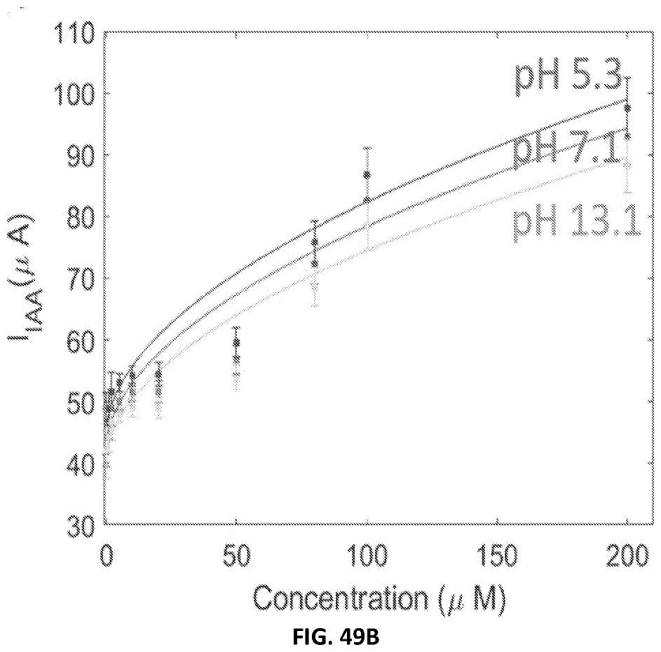

The calibration curves of SA and IAA sensors at different pH values are illustrated in FIGS. 49A-49B.

### Example 20

Real-time Monitoring of Fruit Ripening. The sensor suite, e.g., as shown in an exemplary sensor suite depicted in FIG. 45C, was deployed on bell peppers through a drone. As shown in FIG. 45C, the drone-interfaced plant sensor 50 comprising a sensor suite 51 was attached to the plant leaf 52 and configured to interface with a drone device 53. The sensor suite, as illustrated in 51, was used for multiplexed detection of ethylene, SA, and IAA levels with pH correction on the single platform. As illustrated in FIG. 45C, the sensor suite comprised a working electrode for pH (WE_{pH}) 54, a working electrode for SA (WE_{SA}) 55, a first reference electrode (RE) 56, a first counter electrode (CE) 57, a working electrode for ethylene (WE_{ET}) 58 and 61, a second CE 59, a second RE 60, and a working electrode for IAA (WE_{IAA}) 62.

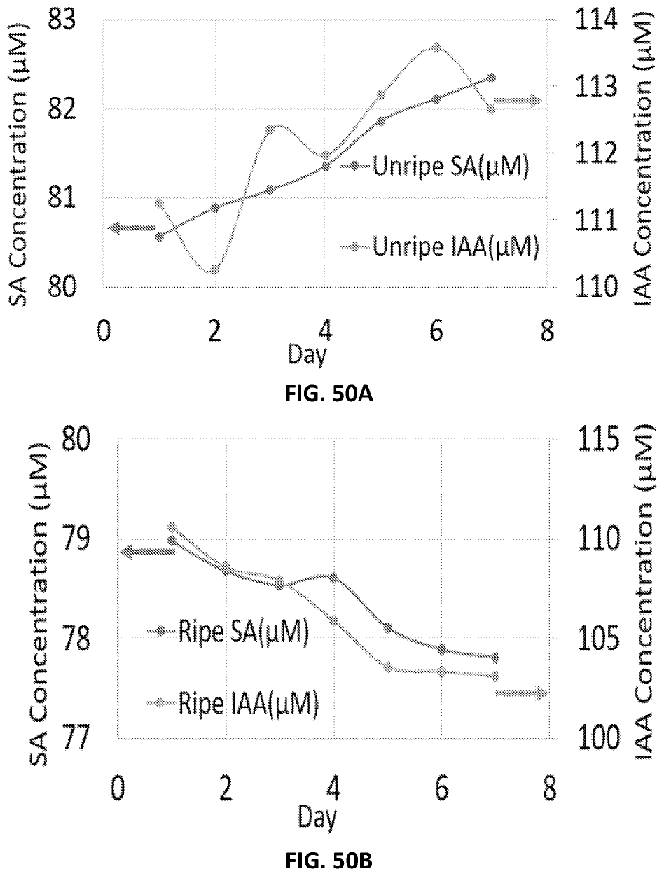

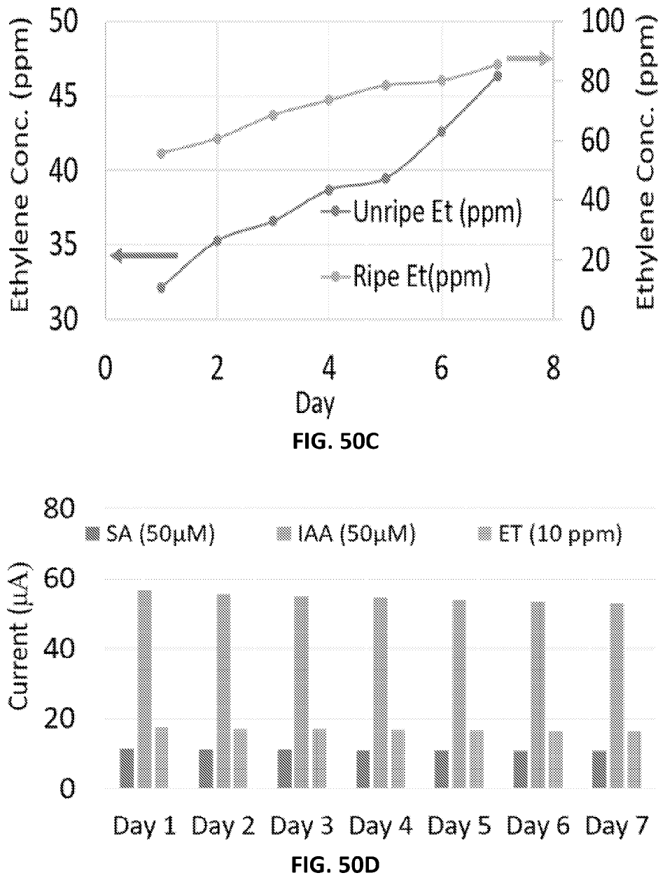

The SA, IAA, and ethylene levels were measured once a day for 7 consecutive days (FIGS. 50A-50C). The results show that both SA and IAA levels increased over time in unripe bell pepper, while the levels started to decrease once the bell pepper reached its maturity. The results are consistent with previous metabolic profiling studies (A. Oikawa, T. Otsuka, R. Nakabayashi, Y. Jikumaru, K. Isuzugawa, H. Murayama, K. Saito, K. Shiratake, “Metabolic profiling of developing pear fruits reveals dynamic variation in primary and secondary metabolites, including plant hormones,” PLoS One, vol. 10, pp. e0131408, 2015. doi: 10.1371/journal.pone.0131408), confirming the novel sensors described herein provide measurements that are consistent with conventional techniques using, for example, CE-TOF MS and LC-QTOF-MS, while, in contrast, being both easily deployable and scalable and providing real-time and continuous assessment of stress responses in plants.

Although the ethylene level showed a rising trend in both ripe and unripe bell peppers, the rate of change was higher in the unripe pepper. Stability analysis of SA, IAA, and ET sensors over one week. Peak current value decreased by 1.15%, 1.33%, and 2.5% for SA, IAA, and ET sensors, respectively (FIG. 50D). Thus, each of the sensors showed excellent stability over the 7 day measurement period.

As described above, the plant sensor was capable of monitoring the varying trend of hormone levels in ripe and unripe bell peppers.

### OTHER EMBODIMENTS

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims. Section headings, the materials, methods, and examples are illustrative only and not intended to be limiting.

Other aspects, advantages, and modifications are within the scope of the following claims.

1. A flexible plant sensor, comprising:
a flexible substrate; and
at least one sensor disposed on the flexible substrate selected from a humidity sensor, a temperature sensor, a strain sensor, a pressure sensor, an electrochemical sensor, or a combination thereof,
wherein the at least one sensor comprises two or more electrodes, or
one or more flexible substrates, and
an array of sensors disposed on the one or more flexible substrates comprising:
i) an electrochemical sensor and at least one of a temperature sensor and a humidity sensor; and
ii) a strain sensor and a pressure sensor.

2. (canceled)

3. The flexible plant sensor of claim 1, wherein the at least one sensor has a coefficient of variance between calibration curves of not more than 3% based on four repeating measurements.

4. (canceled)

5. The flexible plant sensor of claim 1, wherein the at least one sensor has a coefficient of variance between calibration curves of not more than 8% before and after a dynamic folding test, wherein in the dynamic folding test the flexible plant sensor in an unbent orientation is bent to a 90° angle, returned to the unbent orientation, and repeated up to 30 cycles, or 60 cycles, or 90 cycles, or 100 cycles.

6. The flexible plant sensor of claim 1, wherein the at least one sensor has a hysteresis between 0th and 100th cycles of less than 5%.

7. The flexible plant sensor of claim 1, wherein the at least one sensor has a coefficient of variance of <5% up to one hour, or <5% up to 7 days.

8. The flexible plant sensor of claim 1, wherein a) the at least one sensor has a coefficient of variance between calibration curves of not more than 9% over a temperature range of 10° C. to 55° C., or b) the at least one sensor has a coefficient of variance between calibration curves of not more than 9% over a relative humidity (RH) range of 10 RH to 90 RH, or c) both a) and b).

9. (canceled)

10. The flexible plant sensor of claim 1, wherein a) the flexible substrate is a thermoplastic and/or thermosetting film, b) the flexible substrate has a thickness of 50 μm to 500 μm, or 100 μm to 400 μm, or 125 μm to 350 μm, or 150 μm to 250 μm, or 175 μm to 200 μm, c) the flexible plant sensor has a width of 0.1 cm to 2 cm, or 0.25 cm to 1.5 cm, or 0.5 cm to 1 cm, or 0.75 cm, and a length of 0.5 cm to 5 cm, or 0.75 cm to 2.5 cm, or 1 cm to 2 cm, or 1.5 cm, d) the flexible plant sensor is 5 grams or less, e) the flexible plant sensor has a surface area of 1 to 15 cm², or 3 to 12 cm² or 4 to 10 cm², or 6 to 8 cm², and/or f) the at least one sensor further comprises a coating selected from a graphene ink, an Ag/AgCl paste, a metal organic framework (MOF), a poly(3,4-ethylenedioxythiophene) polystyrene sulfonate (PEDOT:PSS) cross-linked with 3-glycidyloxypropyl)trimethoxysilane (GOPS), or a combination thereof.

11. The flexible plant sensor of claim 1, wherein the flexible substrate is a flexible polyimide film, a perfluorinated sulfonic-acid isomer film, or a sulfonated tetrafluoroethylene fluoropolymer-copolymer film.

12. (canceled)

13. (canceled)

14. (canceled)

15. (canceled)

16. (canceled)

17. The flexible plant sensor of claim 10, wherein the metal organic framework comprises at least one metal selected from copper, zinc, or gold.

18. The flexible plant sensor of claim 1, wherein a) the at least one sensor comprises a coating of CuMOF and carbon black (CB) in a weight ratio of 1:3 to 3:1, b) the at least one sensor comprises a coating of a composite copper complex (I)-single-walled carbon nanotube coating, c) wherein the at least one sensor comprises a coating of a functionalized multiwalled carbon nanotube (f-MWCNT) and hydroxyethyl cellulose (HEC) in a weight ratio of 1:6 to 1:1, c) the at least one sensor comprises a coating of Poly(3,4-ethylenedioxythiophene):poly(styrenesulfonate) (PEDOT:PSS) and 3(glycidyloxypropyl)trimethoxysilane (GOPS) in a weight ratio of 1:1 to 1:11, d) the at least one sensor comprises a coating of polydimethylsiloxane (PDMS), deep eutectic solvent (DES), and carbon black (CB) in a weight ratio of 1:1-0.5:0.02-0.04, and/or e) the at least one sensor further comprises an encapsulation layer having a thickness of 0.5 mil to 2 mil.

19. (canceled)

20. (canceled)

21. (canceled)

22. (canceled)

23. (canceled)

24. The flexible plant sensor of claim 1, wherein the at least one sensor is capable of real-time and/or continuous monitoring of one or more physical and/or chemical parameters in a plant.

25. The flexible plant sensor of claim 1, wherein the electrochemical sensor is at least one of a phytohormone sensor or a volatile organic compound sensor.

26. The flexible plant sensor of claim 1, wherein the strain sensor has a gauge factor of at least 800 at a strain of 0.4% to 2% and a curvature angle detection of at least 0.01 degrees and/or has an angle of curvature detection from 5° to 350° or 6° to 320° or 70 to 3000 or 80 to 290°.

27. (canceled)

28. (canceled)

29. (canceled)

30. (canceled)

31. The flexible plant sensor of claim 1, wherein the phytohormone sensor is configured to quantitatively measure the level of at least one of salicylic acid, abscisic acid, jasmonic acid, and indole-3-acetic acid.

32. The flexible plant sensor of claim 1, wherein the pressure sensor has a detection range of 0.1 kPa to 100 kPa.

33. The flexible plant sensor of claim 1, wherein the at least one sensor is bioagent-free and/or non-invasive.

34. (canceled)

35. The flexible plant sensor of claim 1, further comprising a data acquisition system, wherein the data acquisition system comprises a processor;
a communication unit; and a power supply unit, and wherein the data acquisition system is in communication with the at least one sensor.

36. The flexible plant sensor of claim 1, further comprising a potentiostat.

37. The flexible plant sensor of claim 1, wherein the electrochemical sensor comprises a reference electrode (RE), a counter electrode (CE), and at least one working electrode (WE).

38. The flexible plant sensor of claim 35, wherein the data acquisition system comprises a non-transitory computer readable medium communicatively coupled to the processor, the non-transitory computer readable medium having stored thereon computer software comprising a set of instructions that, when executed by the processor, causes the electrode control unit to:
receive electrode data from each of the three or more electrodes; and
send, via the communication unit, the sensor data to an external device.

39. (canceled)

40. (canceled)

41. The flexible plant sensor of claim 1, wherein the flexible plant sensor is configured to select one or more calibration plots to analyze at least one electrode data and/or to perform a signal calibration of at least one sensor.

42. (canceled)

43. The flexible plant sensor of claim 41, wherein the calibration comprises a pH-based signal correction, a temperature-based signal correction, a humidity-based signal correction, a sensor bending correction, a pressure-based signal calibration, a signal calibration based on the signal of an analyte, or a combination thereof.

44. The flexible plant sensor of claim 1, wherein the at least one sensor is configured to attach to a plant leaf, a plant stem, or both separately or concurrently.

45. (canceled)

46. A method for continuously measuring one or more plant conditions, comprising:
attaching to the plant an array of sensors, wherein each sensor is operatively connected to data acquisition system;
measuring at least one signal correction parameter;
determining a value of the one or more plant conditions using a pre-determined calibration plot, wherein the pre-determined calibration plot is based on the measured value of the at least one signal correction parameter.

47-68. (canceled)

69. A method of manufacturing a flexible plant sensor, comprising:
a) attaching a transfer film onto a part of a flexible substrate;
b) forming a pattern in the transfer film attached to the flexible substrate, wherein the pattern is not continuously present in the flexible substrate;
c) removing a portion of the transfer film on the flexible substrate corresponding to the formed pattern to expose a portion of the flexible substrate;
d) applying an ink solution uniformly over the exposed portion of the flexible substrate;
e) curing the dispersion solution at 50° C. to 100° C. for 5 minutes to 60 minutes; and
f) removing the remaining transfer film from the flexible substrate to obtain the flexible plant sensor.

70-82. (canceled)
